SEQUENCE LISTING (296 sequences)

SEQ ID NO: 1 - DNA, 21 bp, Filamentous fungi
  modified_base (6)..(6): I
gayccnmgnttyttyaayatg

SEQ ID NO: 2 - DNA, 20 bp, Filamentous fungi
  modified_base (3)..(3): I
gtnccngtnccrtgcatytc

SEQ ID NO: 3 - DNA, 500 bp, Filamentous fungi
cattaccgagtgagggccctctgggtccaacctcccacccgtgtttatttaccttgttgcttcggcgggcccgccttaactggccgccggggggcttacgcccccgggcccgcgcccgccgaagacaccctcgaactctgtctgaagattgtagtctgagtataaatataaattatttaaaactttcaacaacggatctcttggttccggcatcgatgaagaacgcagcgaaatgcgatacgtaatgtgaattgcaaattcagtgaatcatcgagtctttgaacgcacattgcgccccctggtattccggggggcatgcctgtccgagcgtcattgctgccctcaagcccggcttgtgtgttgggccccgtcctccgattccgggggacgggcccgaaaggcagcggcggcaccgcgtccggtcctcgagcgtatggggctttgtcacccgctctgtaggcccggccggcgcttgccgatcaacccaaatttttatccag

SEQ ID NO: 4 - protein, 28 aa, Penicillium coprobium PF1169
QPWKDSIWAGDVYMFEGDDIVAVYGGVK

SEQ ID NO: 5 - protein, 36 aa, Penicillium coprobium PF1169
HNSIFQALARKILDMALPPGGGAPAPAPAAKRPAPI

SEQ ID NO: 6 - protein, 70 aa, Penicillium coprobium PF1169
GRFLSSDGRCHTFDEKANGYARGEAVGCLILKPLAKALHDQNKIRAVIRGTGSNQDGRTAGITVPNGAAQ

SEQ ID NO: 7 - protein, 74 aa, Penicillium coprobium PF1169
RISYYFDWQGPSMAVDTGCSSSLLAVHLGVEALQNDDCSMAVAVGSNLILSPNAYIADSKTRMLSPTGRSRMWD

SEQ ID NO: 8 - protein, 51 aa, Penicillium coprobium PF1169
SSFLTSTVQQIVEETIQGGTGQVVMESDLMQTEFLEAANGHRMNDCGVVTS

SEQ ID NO: 9 - protein, 79 aa, Penicillium coprobium PF1169
FNAAHRVLPLPSYKWDLKNYWIPYTNNFCLLKGAPAAPVAEATPISVFLSSAAQRVLETSGDNSSAFIVIENDIADPDL

SEQ ID NO: 10 - protein, 84 aa, Penicillium coprobium PF1169
VIRGTGSNQDGRTAGITVPNGAAQESLIRSVYAQADLDPSETDFVEAHGTGTLAGDPVETGAIARVFGTDRPPGDPVRIGSIKT

SEQ ID NO: 11 - protein, 81 aa, Penicillium coprobium PF1169
QEAKAMDPQQRMLLECTYEALENGGISKESLKGQNVGVFVGSAFPDYEMYNRRDLETAPMHQSTGNALALQSNRISYYFDF

SEQ ID NO: 12 - protein, 66 aa, Penicillium coprobium PF1169
NHTGRAEQSKIAIIGLSGRFPEAPDTEAFWDLLKKGLDVHREVPPERWDVKAHVDPEGKKRTPAKL

SEQ ID NO: 13 - protein, 14 aa, Penicillium coprobium PF1169
EKNTSQVEYGCWYN

SEQ ID NO: 14 - protein, 71 aa, Penicillium coprobium PF1169
AGGNTTVALEDAPIRTRSGSDPRSLHPIAISAKSKVSLRGNLENLLAYLDTHPDVSLSDLSYTTTARRHHH

SEQ ID NO: 15 - protein, 77 aa, Penicillium coprobium PF1169
ATDTEKFWDLLASGVDVHRKIPADRFDVETHYDPNGKRMNASHTPYGCFIDEPGLFDAAFFNMSPREAQQTDPMQRL

SEQ ID NO: 16 - protein, 39 aa, Penicillium coprobium PF1169
PEYSQPLCTAIQIALVELLESFGVVPKAVVGHSSGEIAA

SEQ ID NO: 17 - protein, 71 aa, Penicillium coprobium PF1169
RRTFLPWRLTSSALSGQELTQSLAIDAVPIRSSKEPTVGFVFTGQGAQWHGMGKELLSTYPIFRQTMQDVD

SEQ ID NO: 18 - protein, 75 aa, Penicillium coprobium PF1169
LRRLLHAKNDSLVAAFFQKTYCALRKEITSLPPSERQVFPRFTSIVDLLARFKEFGPNPALESALTTIYQLGCFI

SEQ ID NO: 19 - protein, 81 aa, Penicillium coprobium PF1169
FDAAFFNMSPREAQQTDPMQRLAIVTAYEALERAGYVANRTAATNLHRIGTFYGQASDDYREVNTAQEISTYFIPGGCRAF

SEQ ID NO: 20 - protein, 38 aa, Penicillium coprobium PF1169
DTACSSSLVALHYAVQSLRNGESTEALIAGCHLNIVPD

SEQ ID NO: 21 - protein, 75 aa, Penicillium coprobium PF1169
AKHPPATSILLQGNPKTATQSLFLFPDGSGSATSYATIPGISPDVCVYGLNCPYMRTPEKLKFSLDELTAPYVAE

SEQ ID NO: 22 - protein, 38 aa, Penicillium coprobium PF1169
GNGSAMISNRISWFFDLKGPSLSLDTACSSSLVALHLA

SEQ ID NO: 23 - protein, 57 aa, Penicillium coprobium PF1169
AIRDEVRQLPTPLRALVPAFENVLELANYTDLRKGPLSGSIDGVLLCVVQLSSLIGY

SEQ ID NO: 24 - protein, 74 aa, Penicillium coprobium PF1169
AVAWDPQQRILLEVVYEALESAGYFRAGIKPELDDYGCYIGAVMNNYYDNMSCQPTTAYATVGTSRCFLSGCVS

SEQ ID NO: 25 - protein, 52 aa, Penicillium coprobium PF1169
GVIVGSAANQNLNLSHITVPHSGSQVKLYQNVMSQAGVHPHSVTYVEAHGTG

SEQ ID NO: 26 - protein, 57 aa, Penicillium coprobium PF1169
WRITVAIVGGVNALCGPGLTRVLDKAGAISSDGSCKSFDDDAHGYARGEGAGALVLK

SEQ ID NO: 27 - protein, 78 aa, Penicillium coprobium PF1169
LIDDTTVWIEIGPHPVCLGFVKATLESVAVAVPSLRRGENAWCTLAQSLTTLHNAGVPVGWSEFHRPFERALCLLDLP

SEQ ID NO: 28 - protein, 65 aa, Penicillium coprobium PF1169
VWIEIGPHPVCLGFVKATLESVAVAVPSLRRGENAWCTLAQSLTTLHNAGVPVGWSEFHRPFERA

SEQ ID NO: 29 - protein, 83 aa, Penicillium coprobium PF1169
TSDDYREVNSGQDIDTYFIPGGNRAFTPGRINYYFKFSGPSVSVDTACSSSLAAIHVACNSLWRNESDSAVAGGVNILTNPDN

SEQ ID NO: 30 - protein, 54 aa, Penicillium coprobium PF1169
GRFLSSDGRCHTFDEKANGYARGEAVGCLILKPLAKALHDQNKIRAVIRGTGSN

SEQ ID NO: 31 - protein, 63 aa, Penicillium coprobium PF1169
DTACSSSLYALHSACLALDSRDCDGAVVAAANLIQSPEQQMIAVKAGILSPDSMCHTFDESAN

SEQ ID NO: 32 - protein, 55 aa, Penicillium coprobium PF1169
KQTTSRGYFLDHLEDFDCQFFGISPKEAEQMDPQQRVSLEVASEALEDAGIPAKS

SEQ ID NO: 33 - protein, 38 aa, Penicillium coprobium PF1169
PVGCRAFGPGRINYFFKFSGPSFSIDTACSSSLATIQV

SEQ ID NO: 34 - protein, 18 aa, Penicillium coprobium PF1169
ACTSLWNGETDTVVAGGM

SEQ ID NO: 35 - protein, 12 aa, Penicillium coprobium PF1169
TAQEISTYFIPG

SEQ ID NO: 36 - protein, 39 aa, Penicillium coprobium PF1169; identical to SEQ ID NO: 16

SEQ ID NO: 37 - protein, 36 aa, Penicillium coprobium PF1169
ISQPACTALQIALVDLLAEWSITPSVVVGHSSGEIA

SEQ ID NO: 38 - protein, 39 aa, Penicillium coprobium PF1169; identical to SEQ ID NO: 16

SEQ ID NO: 39 - protein, 76 aa, Penicillium coprobium PF1169
EEFWDLCSRGRGAWSPVPKDRFNAGSFYHPNADRPGSFNAAGAHFLTEDIGLFDAPFFNITLQEAQTMDPQQRIFL

SEQ ID NO: 40 - protein, 77 aa, Penicillium coprobium PF1169
INEPRDRPQFFHAHGTGTQAGDPQEAEAVSTALFPDGSNIETKLFVGSIKTVIGHTEGSAGLASLIGSSLAMKHGVI

SEQ ID NO: 41 - protein, 43 aa, Penicillium coprobium PF1169
KLAFVFTGQGGQWAGMGRELLSISTFRESMARSQEILASLGCP

SEQ ID NO: 42 - protein, 71 aa, Penicillium coprobium PF1169
KSFLDDLAFTVNERRSIFPWKAAVVGDTMEGLAASLAQNIKPRSVLRMPTLGFVFTGQGAQWPGMGKELLQ

SEQ ID NO: 43 - protein, 55 aa, Penicillium coprobium PF1169
SVACINSPFNCTLSGPEEDIDAVKAQADQDGLFAQKLKTGVAYHSTAMSAIANDY

SEQ ID NO: 44 - protein, 68 aa, Penicillium coprobium PF1169
MLAVGASASDIQQILDAMRGNKAVIACVNSESSVTLSGDLDVIANLQTALDKEGIFTRKLKVDVAYHS

SEQ ID NO: 45 - protein, 38 aa, Penicillium coprobium PF1169; identical to SEQ ID NO: 22

SEQ ID NO: 46 - protein, 77 aa, Penicillium coprobium PF1169
GPSMTIDTACSSSLIALHQAVQSLRSGETDVAVAAGTNLLLGPEQYIAESKLKMLSPNGRSRMWDKDADGYARGDGI

SEQ ID NO: 47 - protein, 61 aa, Penicillium coprobium PF1169
IGSIKPNIGHLEAGAGVMGFIKAILSIQKGVLAPQANLTKLNSRIDWKTAGVKVVQEATPW

SEQ ID NO: 48 - protein, 37 aa, Penicillium coprobium PF1169
GLFDAPFFNITLQEAQTMDPQQRIFLECVYEALENGG

SEQ ID NO: 49 - protein, 70 aa, Penicillium coprobium PF1169; identical to SEQ ID NO: 6

SEQ ID NO: 50 - protein, 37 aa, Penicillium coprobium PF1169
SFDSRAEGYARGEGVGTVVVKPLSTAIRDGDTIRAVI

SEQ ID NO: 51 - protein, 83 aa, Penicillium coprobium PF1169
WPRLPERRRIAVVNNFSAAGGNTTVALEDAPIRTRSGSDPRSLHPIAISAKSKVSLRGNLENLLAYLDTHPDVSLSDLSYTTT

SEQ ID NO: 52 - protein, 59 aa, Penicillium coprobium PF1169
VYSGSMTNDYELLSTRDIYDMPHNSATGNGRTMLANRLSWFFDLQGPSIMMDTACSSSL

SEQ ID NO: 53 - protein, 67 aa, Penicillium coprobium PF1169
LSPQNNPEDRCQYFEAHGTGTQAGDPQEAAAINSSFFGPESVPDSTDRLYVGSIKTIIGHTEATAGL

SEQ ID NO: 54 - protein, 83 aa, Penicillium coprobium PF1169
DGYGRGEGVASVVLKRLQDAINDGDPIECVIRASGANSDGRTMGITMPNPKAQQSLILATYARAGLSPQNNPEDRCQYFEAHG

SEQ ID NO: 55 - protein, 38 aa, Penicillium coprobium PF1169
MLAVGASASDIQQILDAMRGNKAVIACVNSESSVTLSG

SEQ ID NO: 56 - protein, 29 aa, Penicillium coprobium PF1169
SGCYRELADCPGQRGIFTRKLKVDVAYHS

SEQ ID NO: 57 - protein, 38 aa, Penicillium coprobium PF1169; identical to SEQ ID NO: 22

SEQ ID NO: 58 - protein, 59 aa, Penicillium coprobium PF1169
ISECVTVYWKAIKSAQPDGPYALAGYSYGSMLAFEVAKLLIKNGDKVDFLGCFNLPPHI

SEQ ID NO: 59 - protein, 72 aa, Penicillium coprobium PF1169
GAAVQLVIEGGNQPKGAMMAVGANASTVQPLLDAMKDKHAVVACINSDSSITVSGDETAIEDLESVLKRQDI

SEQ ID NO: 60 - protein, 79 aa, Penicillium coprobium PF1169
SVPIEEHSPVVTQLGTTCVQMALTKYWTSLGVTPSFVMGHSLGEFAALNAAGVLTISDTIYLAGRRAQLLTEQIKVGTH

SEQ ID NO: 61 - protein, 67 aa, Penicillium coprobium PF1169
FIEDSISKEHKPTRVPIHGPYHASHLYNDRDIDRIMESWPTEQLWAYVPQIPVLSTQTGKAFQADSL

SEQ ID NO: 62 - protein, 76 aa, Penicillium coprobium PF1169
GPSMTIDTACSSSLIALHQAVQSLRSGETDVAVAAGTNLLLGPEQYIAESKLKMLSPNGRSRMWDKDADGYARGDG

SEQ ID NO: 63 - protein, 31 aa, Penicillium coprobium PF1169
LFLFPDGSGSATSYATIPGISPDVCVYGLNC

SEQ ID NO: 64 - protein, 26 aa, Penicillium coprobium PF1169
AKHPPATSILLQGNPKTATQSFIFVP

SEQ ID NO: 65 - protein, 46 aa, Penicillium coprobium PF1169
YQATGCAASLQSNRISYFFDLRGPSITIDTACSSSLVALHYAVQSL

SEQ ID NO: 66 - protein, 66 aa, Penicillium coprobium PF1169
YSATGSGLTVLANRITHCFDLRGPSHVVDTACSSSLYALHSACLALDSRDCDGAVVAAANLIQSPE

SEQ ID NO: 67 - protein, 76 aa, Penicillium coprobium PF1169
SVPIEEHSPVVTQLGTTCVQMALTKYWTSLGVTPSFVMGHSLGEFAALNAAGVLTISDTIYLAGRRAQLLTEQIKV

SEQ ID NO: 68 - protein, 71 aa, Penicillium coprobium PF1169
HLNLMGPSTAVDAACASSLVAIHHGVQAIKLGESRVAIVGGVNALCGPGLTRVLDKAGSISSDGSCKSFDD

SEQ ID NO: 69 - protein, 84 aa, Penicillium coprobium PF1169
SFRRQEDTWKVLSNATSTLYLAGIEIKWKEYHQDFNAAHRVLPLPSYKWDLKNYWIPYTNNFCLLKGAPAAPVAEATPISVFLS

SEQ ID NO: 70 - protein, 78 aa, Penicillium coprobium PF1169
KTSCFVGSFSADYTDLLLRDPECVPMYQCTNAGQSRAMTANRLSYFFDLKGPSVTVDTACSGSLVALHLACQSLRTGD

SEQ ID NO: 71 - protein, 67 aa, Penicillium coprobium PF1169
YSATGSGLTVLANRITHCFDLRGPSHVVDTACSSSLYALHSACFGPLNSRDCDGAVVAAANLIQSPE

SEQ ID NO: 72 - protein, 79 aa, Penicillium coprobium PF1169
SVPIEEHSPVVTQLGTTCVQMALTKYWTSLGVTPSFVMGHSLGEFAALNAAGVLTISDTIYLAGRRAQLLTEQIEGGTH

SEQ ID NO: 73 - protein, 40 aa, Penicillium coprobium PF1169
EANLHVPLEPTPWPAGRPERISVNSFGIGGSNAHAILESA

SEQ ID NO: 74 - protein, 70 aa, Penicillium coprobium PF1169
  misc_feature (5)..(5): Xaa can be any naturally occurring amino acid
IGHTXGSAGLASLIGSSLAMKHGVIPPNLHFGQLSEKVAPFYTHLNIPTEPVPWPNSTSSQVKRASINSF

SEQ ID NO: 75 - protein, 45 aa, Penicillium coprobium PF1169
PVCSGMVKATFGPQATTVASFRRQEDTWKVLSNATSTLYLAGIEI

SEQ ID NO: 76 - protein, 19 aa, Penicillium coprobium PF1169
  misc_feature (13)..(13): Xaa can be any naturally occurring amino acid
LLGLRLKWKEYHXDFNAAH

SEQ ID NO: 77 - protein, 69 aa, Penicillium coprobium PF1169
VYSGSMTNDYELLSTRDIYDMPHNSATGNGRTMLANRLSWFFDLQGPSIMMDTACSSSLTAVHLAAQSL

SEQ ID NO: 78 - protein, 85 aa, Penicillium coprobium PF1169
DAQFFGTKPVEANSIDPQQRLLLETVYEGLETSGIPMERLQGSNTAVYVGLMTNDYADMLGRDMQNFPTYFASGTARSILSNRVS

SEQ ID NO: 79 - protein, 28 aa, Penicillium coprobium PF1169
DPAYFDSSFFNITKTELLTLDPQQRLVL

SEQ ID NO: 80 - protein, 51 aa, Penicillium coprobium PF1169
VACVNSPASTTLSGDVDYINQLEARLQQDGHFARKLRIDTAYHSPHMEELV

SEQ ID NO: 81 - protein, 24 aa, Penicillium coprobium PF1169
LKSISPVVTQLGTTCVQMALTKYW

SEQ ID NO: 82 - protein, 59 aa, Penicillium coprobium PF1169
GCFYGMTSDDYREVNSGQDIDTYFIPGGNRAFTPGRINYYFKFSGPSVSVDTACSSSLA

SEQ ID NO: 83 - protein, 16 aa, Penicillium coprobium PF1169
LEMAGFIPDSIPLRRR

SEQ ID NO: 84 - protein, 53 aa, Penicillium coprobium PF1169
AIVGGVNALCGPGLTRVLDKAGAISSDGSCKSFDDDAHGYARGEGAGALVTKK

SEQ ID NO: 85 - protein, 60 aa, Penicillium coprobium PF1169
IAIVGIGGRFPGEATNPNRLWDMVSNGRSALTEVPKDRFNIDAFYHPHAERQGTMNVRRG

SEQ ID NO: 86 - protein, 53 aa, Penicillium coprobium PF1169
SVPIEEHSPVVTQLGTTCVQMALTKYWTSLGVTPSFVMGHSLGEFAALNAAGV

SEQ ID NO: 87 - protein, 18 aa, Penicillium coprobium PF1169
SVPIEEHSPVVTQLGTTC

SEQ ID NO: 88 - protein, 62 aa, Penicillium coprobium PF1169
FLDDLAFTVNERRSIFPWKAAVVGDTMEGLAASLAQNIKPRSVLRMPTLGFVFTGQGAQWPG

SEQ ID NO: 89 - protein, 51 aa, Penicillium coprobium PF1169; identical to SEQ ID NO: 8

SEQ ID NO: 90 - protein, 77 aa, Penicillium coprobium PF1169
ECGFVEMHGTGTKAGDPVEAAAVHAALGKNRTLRNPLYIGSVKSNIGHLEGASGIVAVIKAAMMLDRDLMLPNAEFK

SEQ ID NO: 91 - protein, 78 aa, Penicillium coprobium PF1169
  misc_feature (4)..(4): Xaa can be any naturally occurring amino acid
FFKXSGPSFSIDTACSSSLATIQVCTHLFHVHLNRQLTIAACTSLWNGETDTVVAGGMNILTNSDAFAGLSHGHFLTK

SEQ ID NO: 92 - protein, 79 aa, Penicillium coprobium PF1169; identical to SEQ ID NO: 72

SEQ ID NO: 93 - protein, 68 aa, Penicillium coprobium PF1169
LSSDGRCHTFDEKANGYARGEAVGCLILKPLAKALHDQNKIRAVIRGTGSNQDGRTAGITVPNGAAQE

SEQ ID NO: 94 - protein, 80 aa, Penicillium coprobium PF1169
SPLFGLARIIASEHPDLGSLIDIEEPIIPLSTMRYIQGADIVRISDGIARTSRFRSLPRTKLRPVSDGPRLLPRPEGTYL

SEQ ID NO: 95 - protein, 75 aa, Penicillium coprobium PF1169
NRISYYFDWQGPSMAVDTGCSSSLLAVHLGVEALQNDDCSMAVAVGSNLILSPNAYIADSKTRMLSPTGRSRMWD

SEQ ID NO: 96 - protein, 81 aa, Penicillium coprobium PF1169
VDVNPAVLKDAPLPWDPSSWAPILDAATSVGSTIFQTAALRMPAQIERVEIFTSENPPKTSWLYVQEASDAVPTSHVSVVS

SEQ ID NO: 97 - protein, 37 aa, Penicillium coprobium PF1169
PLFGLARIIASEHPDLGSLIDIEEPIIPLSTMRYIRG

SEQ ID NO: 98 - protein, 84 aa, Penicillium coprobium PF1169
AVIRGTGSNQDGRTAGITVPNGAAQESLIRSVYAQADLDPSETDFVEAHGTGTLAGDPVETGAIARVFGTDRPPGDPVRIGSIK

SEQ ID NO: 99 - protein, 69 aa, Penicillium coprobium PF1169
LEVVWECLENSGETQWRGKEIGCFVGVFGEDWLEMSHKDPQHLNQMFPIATGGFALANQVSYRFDLTGP

SEQ ID NO: 100 - protein, 79 aa, Penicillium coprobium PF1169
GGATDTEKFWDLLASGVDVHRKIPADRFDVETHYDPNGKRMNASHTPYGCFIDEPGLFDAAFFNMSPREAQQTDPMQRL

SEQ ID NO: 101 - protein, 52 aa, Penicillium coprobium PF1169
ELRHGKNIDKPEYSQPLCTAIQIALVELLESFGVVPKAVVGHSSGEIAAAYV

SEQ ID NO: 102 - protein, 34 aa, Penicillium coprobium PF1169
VGFVFTGQGAQWHGMGKELLSTYPIFRQTMQDVD

SEQ ID NO: 103 - protein, 63 aa, Penicillium coprobium PF1169
FDAAFFNMSPREAQQTDPMQRLAIVTAYEALERAGYVANRTAATNLHRIGTFYGQASDDYREV

SEQ ID NO: 104 - protein, 43 aa, Penicillium coprobium PF1169
AVVSGVSILENPVETIGMSHHGLLGPQGRSFSFDSRAEGYARG

SEQ ID NO: 105 - protein, 71 aa, Penicillium coprobium PF1169
KASLSLQHGMIAPNLLMQHLNPKIKPFAAKLSVPTECIPWPAVPDGCPRRASVNSFGFGGANVHVVLESYT

SEQ ID NO: 106 - protein, 28 aa, Penicillium coprobium PF1169
PWPTTGLRRASVNSFGYGGTNAHCVLDD

SEQ ID NO: 107 - protein, 71 aa, Penicillium coprobium PF1169; identical to SEQ ID NO: 105

SEQ ID NO: 108 - protein, 50 aa, Penicillium coprobium PF1169
DRLFLQMSHEEWEAALAPKVTGTWNLHHATAQHSLDFFVVFGSIAGVCGN

SEQ ID NO: 109 - protein, 82 aa, Penicillium coprobium PF1169
TFLKGTGGQMLQNVVLRVPVAINAPRSVQVVVQQDQVKVVSRLIPSEASVLDDDASWVTHTTAYWDRRVLGSEDRIDLAAVK

SEQ ID NO: 110 - protein, 30 aa, Penicillium coprobium PF1169
DTACSSSLVALHYAVQSLRNGESTEALIAG

SEQ ID NO: 111 - protein, 38 aa, Penicillium coprobium PF1169
GTGNGSAMISNRISWFFDLKGPSLSLDTACSSSLVALH

SEQ ID NO: 112 - protein, 72 aa, Penicillium coprobium PF1169
TSTQLNDLNETNAIKKVFGKQAYNIPISSTKSYTGHLIGAAGTMETIFCIKTMQEKIAPATTNLKERDSNCD

SEQ ID NO: 113 - protein, 50 aa, Penicillium coprobium PF1169
VIVGSAANQNLNLSHITVPHSGSQVKLYQNVMSQAGVHPHSVTYVEAHGT

SEQ ID NO: 114 - protein, 48 aa, Penicillium coprobium PF1169
LPTAIQPLFRANVSYLLVGGLGGIGKEVALWMVQNGAKSLIFVNRSGL

SEQ ID NO: 115 - protein, 53 aa, Penicillium coprobium PF1169
VAIVGGVNALCGPGLTRVLDKAGAISSDGSCKSFDDDAHGYARGEGAGALVLK

SEQ ID NO: 116 - protein, 28 aa, Penicillium coprobium PF1169
PWESPGARRVSVNSFGYGGSNAHVIIED

SEQ ID NO: 117 - protein, 72 aa, Penicillium coprobium PF1169
KTLREWMTAEGKDHNLSDILTTLATRRDHHDYRAALVVDDNRDAELALQALEHGVDQTFTTQSRVFGADISK

SEQ ID NO: 118 - protein, 80 aa, Penicillium coprobium PF1169
SDDYREVNSGQDIDTYFIPGGNRAFTPGRINYYFKFSGPSVSVDTACSSSLAAIHVACNSLWRNESDSAVAGGVNILTNP

SEQ ID NO: 119 - protein, 56 aa, Penicillium coprobium PF1169
LSSDGRCHTFDEKANGYARGEAVGCLILKPLAKALHDQNKIRAVIRGTGSNQGRAN

SEQ ID NO: 120 - protein, 63 aa, Penicillium coprobium PF1169; identical to SEQ ID NO: 31

SEQ ID NO: 121 - protein, 28 aa, Penicillium coprobium PF1169
PWPTTGLRRASVNSFGYGGTNAHCVLDD

SEQ ID NO: 122 - protein, 62 aa, Penicillium coprobium PF1169
AGIPLANIMGTKTSCFVGSFSADYTDLLLRDPECVPMYQCTNAGQSRAMTANRLSYFLIKGP

SEQ ID NO: 123 - protein, 80 aa, Penicillium coprobium PF1169
RWEPYYRRDPRNEKFLKQTTSRGYFLDHLEDFDCQFFGISPKEAEQMDPQQRVSLEVASEALEDAGIPAKSLSGSDTAVF

SEQ ID NO: 124 - protein, 28 aa, Penicillium coprobium PF1169
PGRINYFFKFSGPSFSIDTACSSSLATI

SEQ ID NO: 125 - protein, 64 aa, Penicillium coprobium PF1169
AGIPLANIMGTKTSCFVGSFSADYTDLLLRDPECVPMYQCTNAGQSRAMTANRLSYFFDLKGPS

SEQ ID NO: 126 - protein, 52 aa, Penicillium coprobium PF1169; identical to SEQ ID NO: 101

SEQ ID NO: 127 - protein, 38 aa, Penicillium coprobium PF1169
QPLCTAIQIALVELLESFGVVPKAVVGHSSGEIAAAYV

SEQ ID NO: 128 - protein, 86 aa, Penicillium coprobium PF1169
RLPGDVSTPEEFWDLCSRGRGAWSPVPKDRFNAGSFYHPNADRPGSFNAAGAHFLTEDIGLFDAPFFNITLQEAQTMDPQQRIFLE

SEQ ID NO: 129 - protein, 69 aa, Penicillium coprobium PF1169
QFFHAHGTGTQAGDPQEAEAVSTALFPDGSNIETKLFVGSIKTVIGHTEGSAGLASLIGSSLAMKHGVI

SEQ ID NO: 130 - protein, 64 aa, Penicillium coprobium PF1169; identical to SEQ ID NO: 125

SEQ ID NO: 131 - protein, 67 aa, Penicillium coprobium PF1169
LDDLAFTVNERRSIFPWKAAVVGDTMEGLAASLAQNIKPRSVLRMPTLGFVFTGQGAQWPGMGKELL

SEQ ID NO: 132 - protein, 21 aa, Penicillium coprobium PF1169
AHGTGTKVGDPMEVEAIADVF

SEQ ID NO: 133 - protein, 71 aa, Penicillium coprobium PF1169
KGGMLAVGASASDIQQILDAMRGNKAVIACVNSESSVTLSGDLDVIANLQTALDKEGIFTRKLKVDVAYHS

SEQ ID NO: 134 - protein, 75 aa, Penicillium coprobium PF1169
LENLETALARNAPIYAEVTGYANYSDAYDITAPADDLMGRYMSITKAIEQAQLNINEIDYINAHGTSTQLNDLNE

SEQ ID NO: 135 - protein, 53 aa, Penicillium coprobium PF1169
MAMKKALKQAQLRPSAVDYVNAHATSTIVGDAAENAAIKALLLGADGKDKAAD

SEQ ID NO: 136 - protein, 38 aa, Penicillium coprobium PF1169; identical to SEQ ID NO: 111

SEQ ID NO: 137 - protein, 76 aa, Penicillium coprobium PF1169; identical to SEQ ID NO: 62

SEQ ID NO: 138 - protein, 85 aa, Penicillium coprobium PF1169
IGSIKPNIGHLEAGAGVMGFIKAILSIQKGVLAPQANLTKLNSRIDWKTAGVKVVQEATPWPSSDSIRRAGVCSYGYGGTVSHAV

SEQ ID NO: 139 - protein, 57 aa, Penicillium coprobium PF1169
NAAGAHFLTEDIGLFDAPFFNITLQEAQTMDPQQRIFLECVYEALENGGIPTHEITG

SEQ ID NO: 140 - protein, 68 aa, Penicillium coprobium PF1169; identical to SEQ ID NO: 93

SEQ ID NO: 141 - protein, 37 aa, Penicillium coprobium PF1169; identical to SEQ ID NO: 50

SEQ ID NO: 142 - protein, 72 aa, Penicillium coprobium PF1169
GIPIDTLPGSNTAVYSGSMTNDYELLSTRDIYDMPHNSATGNGRTMLANRLSWFFDLQGPSIMMDTACSSSL

SEQ ID NO: 143 - protein, 83 aa, Penicillium coprobium PF1169
AQQSLILATYARAGLSPQNNPEDRCQYFEAHGTGTQAGDPQEAAAINSSFFGPESVPDSTDRLYVGSIKTIIGHTEATAGLAG

SEQ ID NO: 144 - protein, 69 aa, Penicillium coprobium PF1169
PLWRKIETAPLNTGLTHDVEKHTLLGQRIPVAGTDTFVYTTRLDNETKPFPGSHPLHGTEIVPAAGLIN

SEQ ID NO: 145 - protein, 64 aa, Penicillium coprobium PF1169; identical to SEQ ID NO: 125

SEQ ID NO: 146 - protein, 81 aa, Penicillium coprobium PF1169
GYGRGEGVASVVLKRLQDAINDGDPIECVIRASGANSDGRTMGITMPNPKAQQSLILATYARAGLSPQNNPEDRCQYFEAH

SEQ ID NO: 147 - protein, 38 aa, Penicillium coprobium PF1169; identical to SEQ ID NO: 111

SEQ ID NO: 148 - protein, 53 aa, Penicillium coprobium PF1169
EATSMDAQQRKLLEVTYEALENAGVPLETIQGSNTGVYVGNFTNDFLNMQYKD

SEQ ID NO: 149 - protein, 82 aa, Penicillium coprobium PF1169
GSLIDIEEPIIPLSTMRYIQGADIVRISDGIARTSRFRSLPRTKLRPVSDGPRLLPRPEGTYLITGGLGILGLEVADFLVEK

SEQ ID NO: 150 - protein, 65 aa, Penicillium coprobium PF1169
QLGTTCVQMALTKYWTSLGVTPSFVMGHSLGEFAALNAAGVLTISDTIYLAGRRAQLLTEQIKVG

SEQ ID NO: 151 - protein, 78 aa, Penicillium coprobium PF1169
  misc_feature (45)..(45): Xaa can be any naturally occurring amino acid
GPRLLPRPEGTYLITGGLGILGLEVADFLVEKGARRVLLISRRAXPPRRTWDQVATEFQPAITKIRLLESRGASVYVL

SEQ ID NO: 152 - protein, 76 aa, Penicillium coprobium PF1169; identical to SEQ ID NO: 62

SEQ ID NO: 153 - protein, 38 aa, Penicillium coprobium PF1169
NRISYFFDLRGPSITIDTACSSSLVALHYAVQSLRNGE

SEQ ID NO: 154 - protein, 74 aa, Penicillium coprobium PF1169
GSGLTVLANRITHCFDLRGPSHVVDTACSSSLYALHSACLALDSRDCDGAVVAAANLIQSPEQQMIAVKAGILS

SEQ ID NO: 155 - protein, 57 aa, Penicillium coprobium PF1169
QLGTTCVQMALTKYWTSLGVTPSFVMGHSLGEFAALNAAGVLTISDTIYLAGRRAQL

SEQ ID NO: 156 - protein, 72 aa, Penicillium coprobium PF1169
HLNLMGPSTAVDAACASSLVAIHHGVQAIKLGESRVAIVGGVNALCGPGLTRVLDKAGSISSDGSCKSFDDD

SEQ ID NO: 157 - protein, 81 aa, Penicillium coprobium PF1169
LKGTGGQMLQNVVLRVPVAINAPRSVQVVVQQDQVKVVSRLIPSEASVLDDDASWVTHTTAYWDRRVLGSEDRIDLAAVKS

SEQ ID NO: 158 - protein, 82 aa, Penicillium coprobium PF1169
IMGTKTSCFVGSFSADYTDLLLRDPECVPMYQCTNAGQSRAMTANRLSYFFDLKGPSVTVDTACSGSLVALHLACQSLRTGD

SEQ ID NO: 159 - protein, 75 aa, Penicillium coprobium PF1169
GSGLTVLANRITHCFDLRGPSHVVDTACSSSLYALHSACFGPLNSRDCDGAVVAAANLIQSPEQQMIAVKRDSIA

SEQ ID NO: 160 - protein, 28 aa, Penicillium coprobium PF1169
PWPTTGLRRASVNSFGYGGTNAHCVLDD

SEQ ID NO: 161 - protein, 64 aa, Penicillium coprobium PF1169
QLGTTCVQMALTKYWTSLGVTPSFVMGHSLGEFAALNAAGVLTISDTIYLAGRRAQLLTEQIEG

SEQ ID NO: 162 - protein, 60 aa, Penicillium coprobium PF1169
IAPNIHFKMPNPQIPFNEANLHVPLEPTPWPAGRPERISVNSFGIGGSNAHAILESASTV

SEQ ID NO: 163 - protein, 34 aa, Penicillium coprobium PF1169
GLVNILRSWGIEPSTVVGHSSGEIVAAYTARAIS

SEQ ID NO: 164 - protein, 51 aa, Penicillium coprobium PF1169
PWPSEGLRRISVNSFGFGGSNTHVILDDALHYMQQRGLTGNHCTARLPGIL

SEQ ID NO: 165 - protein, 71 aa, Penicillium coprobium PF1169
  misc_feature (5)..(5): Xaa can be any naturally occurring amino acid
IGHTXGSAGLASLIGSSLAMKHGVIPPNLHFGQLSEKVAPFYTHLNIPTEPVPWPNSTSSQVKRASINSFG

SEQ ID NO: 166 - protein, 74 aa, Penicillium coprobium PF1169
GSNTAVYSGSMTNDYELLSTRDIYDMPHNSATGNGRTMLANRLSWFFDLQGPSIMMDTACSSSLTAVHLAAQSL

SEQ ID NO: 167 - protein, 85 aa, Penicillium coprobium PF1169; identical to SEQ ID NO: 78

SEQ ID NO: 168 - protein, 60 aa, Penicillium coprobium PF1169
VVACVNSPASTTLSGDVDYINQLEARLQQDGHFARKLRIDTAYHSPHMEELVGVVGDAIS

SEQ ID NO: 169 - protein, 56 aa, Penicillium coprobium PF1169
FYGMTSDDYREVNSGQDIDTYFIPGGNRAFTPGRINYYFKFSGPSVSVDTACSSSL

SEQ ID NO: 170 - protein, 53 aa, Penicillium coprobium PF1169
VAIVGGVNALCGPGLTRVLDKAGAISSDGSCKSFDDDAHGYARGEGAGALVTK

SEQ ID NO: 171 - protein, 40 aa, Penicillium coprobium PF1169
QLGTTCVQMALTKYWTSLGVTPSFVMGHSLGEFAALNAAG

SEQ ID NO: 172 - protein, 69 aa, Penicillium coprobium PF1169
REWMTAEGKDHNLSDILTTLATRRDHHDYRAALVVDDNRDAELALQALEHGVDQTFTTQSRVFGADISK

SEQ ID NO: 173 - protein, 51 aa, Penicillium coprobium PF1169; identical to SEQ ID NO: 164

SEQ ID NO: 174 - protein, 71 aa, Penicillium coprobium PF1169
FVEMHGTGTKAGDPVEAAAVHAALGKNRTLRNPLYIGSVKSNIGHLEGASGIVAVIKAAMMLDRDLMLPNA

SEQ ID NO: 175 - protein, 41 aa, Penicillium coprobium PF1169
LAIVGMACRLPGQITTPQELWELCSRGRSAWSEIPPERFNP

SEQ ID NO: 176 - protein, 64 aa, Penicillium coprobium PF1169; identical to SEQ ID NO: 161

SEQ ID NO: 177 - protein, 74 aa, Penicillium coprobium PF1169
GASVYVLALDITKPDAVEQLSTALDRLALPSVQGVVHAAGVLDNELVMQTTQEAFNRVLAPKIAGALALHEPFP

SEQ ID NO: 178 - protein, 72 aa, Penicillium coprobium PF1169
GLVNILRSWGIEPSTVVGHSSGEIVAAYTARAISMRTAIILAYYRGKVAQPLEGLGAMVAVGLSPDEVAQYM

SEQ ID NO: 179 - protein, 70 aa, Penicillium coprobium PF1169; identical to SEQ ID NO: 6

SEQ ID NO: 180 - protein, 51 aa, Penicillium coprobium PF1169; identical to SEQ ID NO: 8

SEQ ID NO: 181 - protein, 64 aa, Penicillium coprobium PF1169
LLGLRLKWKEYHQDFNAAHRVLPLPSYKWDLKNYWIPYTNNFCLLKGAPAAPVAEATPISVFLS

SEQ ID NO: 182 - protein, 26 aa, Penicillium coprobium PF1169
SFRRQEDTWKVLSNATSTLYLAGIEI

SEQ ID NO: 183 - protein, 65 aa, Penicillium coprobium PF1169
AGGNTTVALEDAPIRTRSGSDPRSLHPIAISAKSKVSLRGNLENLLAYLDTHPDVSLSDLSYTTT

SEQ ID NO: 184 - protein, 96 aa, Penicillium coprobium PF1169
FDAAFFNMSPREAQQTDPMQRLAIVTAYEALERAGYVANRTAATNLHRIGTFYGQASDDYREVNTAQEISTYFIPGGCRAFGPGRINYFFKFLGPA

SEQ ID NO: 185 - protein, 58 aa, Penicillium coprobium PF1169
FLQISGPSFSIDTACSSSLATIQVCTHLFHVHLNRQLTIAACTSLWNGETDTVVAGGM

SEQ ID NO: 186 - protein, 59 aa, Penicillium coprobium PF1169; identical to SEQ ID NO: 52

SEQ ID NO: 187 - protein, 31 aa, Penicillium coprobium PF1169; identical to SEQ ID NO: 63

SEQ ID NO: 188 - protein, 26 aa, Penicillium coprobium PF1169
AKHPPATSILLQGNPKTATQSFIFVP

SEQ ID NO: 189 - protein, 38 aa, Penicillium coprobium PF1169; identical to SEQ ID NO: 22

SEQ ID NO: 190 - protein, 76 aa, Penicillium coprobium PF1169
AIHHGVQAIKLGESRVAIVGGVNALCGPGLTRVLDKAGAISSDGSCKSFDDDAHGYARGEGAGALVLKSLHQALLD

SEQ ID NO: 191 - protein, 65 aa, Penicillium coprobium PF1169; identical to SEQ ID NO: 28

SEQ ID NO: 192 - protein, 53 aa, Penicillium coprobium PF1169
TSDDYREVNSGQDIDTYFIPGGNRAFTPGRINYYFKFSGPSVSVDTACSSSLA

SEQ ID NO: 193 - protein, 40 aa, Penicillium coprobium PF1169
VDTACSSSLYALHSACFGPLNSRDCDGAVVAAANLIQSPE

SEQ ID NO: 194 - protein, 68 aa, Penicillium coprobium PF1169; identical to SEQ ID NO: 44

SEQ ID NO: 195 - protein, 62 aa, Penicillium coprobium PF1169; identical to SEQ ID NO: 88

SEQ ID NO: 196 - protein, 76 aa, Penicillium coprobium PF1169; identical to SEQ ID NO: 62

SEQ ID NO: 197 - protein, 79 aa, Penicillium coprobium PF1169; identical to SEQ ID NO: 72

SEQ ID NO: 198 - protein, 40 aa, Penicillium coprobium PF1169
FNLKGISQSIASACATSADAIGYAFHLIAAGKQDLMLAGG

SEQ ID NO: 199 - protein, 70 aa, Penicillium coprobium PF1169; identical to SEQ ID NO: 6

SEQ ID NO: 200 - protein, 284 aa, Penicillium coprobium PF1169
LSVKRVGIHDDFFELGGHSLLAVKLVNHLKKVFGTELSVALLAQYSTVESLGEIIRENKEIKPSIVIELRSGTYEQPLWLFHPIGGSTFCYMELSRHLNPNRTLRAIQSPGLIEADAAEVAIEEMATLYIAEMQKMQPQGPYFLGGWCFGGAIAYEISRQLRQMGQQVTGIVMIDTRAPIPENVPEDADDAMLLSWFARDLAVPYGKKLTISAQYLRELSPDHMFDHVLKEAKAINVIPLDANPSDFRLYFDTYLANGVALQTYFPEPEDFPILLVKAKDESED

SEQ ID NO: 201 - protein, 73 aa, Penicillium coprobium PF1169
PMNKDKVYWSAIIRTLVAKEMRVEPETIDPEQKFTTYGLDSIVALSVSGDLEDLTKLELEPTLLWDYPTINAL

SEQ ID NO: 202 - protein, 63 aa, Penicillium coprobium PF1169
GSLIDIEEPIIPLSTMRYIQGADIVRISDGIARTSRFRSLPRTKLRPVSDGPRLLPRPEGTYL

SEQ ID NO: 203 - protein, 69 aa, Penicillium coprobium PF1169; identical to SEQ ID NO: 99

SEQ ID NO: 204 - protein, 96 aa, Penicillium coprobium PF1169; identical to SEQ ID NO: 184

SEQ ID NO: 205 - protein, 58 aa, Penicillium coprobium PF1169; identical to SEQ ID NO: 185

SEQ ID NO: 206 - protein, 52 aa, Penicillium coprobium PF1169; identical to SEQ ID NO: 101

SEQ ID NO: 207 - protein, 59 aa, Penicillium coprobium PF1169; identical to SEQ ID NO: 52

SEQ ID NO: 208 - protein, 28 aa, Penicillium coprobium PF1169
PWPTTGLRRASVNSFGYGGTNAHCVLDD

SEQ ID NO: 209 - protein, 71 aa, Penicillium coprobium PF1169; identical to SEQ ID NO: 105

SEQ ID NO: 210 - protein, 80 aa, Penicillium coprobium PF1169
LKGTGGQMLQNVVLRVPVAINAPRSVQVVVQQDQVKVVSRLIPSEASVLDDDASWVTHTTAYWDRRVLGSEDRIDLAAVK

SEQ ID NO: 211 - protein, 38 aa, Penicillium coprobium PF1169; identical to SEQ ID NO: 22

SEQ ID NO: 212 - protein, 76 aa, Penicillium coprobium PF1169; identical to SEQ ID NO: 190

SEQ ID NO: 213 - protein, 69 aa, Penicillium coprobium PF1169; identical to SEQ ID NO: 172

SEQ ID NO: 214 - protein, 53 aa, Penicillium coprobium PF1169; identical to SEQ ID NO: 192

SEQ ID NO: 215 - protein, 63 aa, Penicillium coprobium PF1169
AGIPLANIMGTKTSCFVGSFSADYTDLLLRDPECVPMYQCTNAGQSRAMTANRLSYFFDLKGP

SEQ ID NO: 216 - protein, 68 aa, Penicillium coprobium PF1169; identical to SEQ ID NO: 44

SEQ ID NO: 217 - protein, 39 aa, Penicillium coprobium PF1169
NAAGAHFLTEDIGLFDAPFFNITLQEAQTMDPQQRIFLE

SEQ ID NO: 218 - protein, 76 aa, Penicillium coprobium PF1169; identical to SEQ ID NO: 62

SEQ ID NO: 219 - protein, 61 aa, Penicillium coprobium PF1169
GLVNILRSWGIEPSTVVGHSSGEIVAAYTARAISMRTAIILAYYRGKVAQPLEGLGAMVAV

SEQ ID NO: 220 - protein, 79 aa, Penicillium coprobium PF1169; identical to SEQ ID NO: 72

SEQ ID NO: 221 - protein, 81 aa, Penicillium coprobium PF1169
VYTGRISLKDLGMRCLPLCLFLFLWTIYFNTAYSYQDIKDDCKLNVNSSYVLAGSHVRGMLLLQAIAVVLVIPWILYTSAS

SEQ ID NO: 222 - protein, 82 aa, Penicillium coprobium PF1169
RHFGLWDEPRELEDVEFLLKADVRNNSAWNHRYMLRFGPRDTSLPDAGMVNAGDLSTAPAEKGRLSVVDEDMVDGELKFAQE

SEQ ID NO: 223 - protein, 35 aa, Penicillium coprobium PF1169
IMRGAGCAINDLWDRNLDPHVERTKFRPIARGALS

SEQ ID NO: 224 - protein, 86 aa, Penicillium coprobium PF1169
FPTFPPKEADFLMEMFAQDSKNYHVWTYRHWLVRHFGLWDEPRELEDVEFLLKADVRNNSAWNHRYMLRFGPRDTSLPDAGMVNAG

SEQ ID NO: 225 - protein, 82 aa, Penicillium coprobium PF1169
NHRYMLRFGPRDTSLPDAGMVNAGDLSTAPAEKGRLSVVDEDMVDGELKFAQEAILRAPENRSPWWYARGVLRAAGRGLGEW

SEQ ID NO: 226 - protein, 45 aa, Penicillium coprobium PF1169
RPTSRKLGVYPQYILGASSALTILPAWASVYTGRISLKDLGMRCL

SEQ ID NO: 227 - DNA, 20 bp, Artificial Sequence
  a primer sequence for PCR
tacaggcggcctaaattgtc

SEQ ID NO: 228 - DNA, 20 bp, Artificial Sequence
  a primer sequence for PCR
gaacacagcgcaagagatca

SEQ ID NO: 229 - DNA, 20 bp, Artificial Sequence
  a primer sequence for PCR
cgcaagacttgaggaacaag

SEQ ID NO: 230 - DNA, 20 bp, Artificial Sequence
  a primer sequence for PCR
tgaggtcaacagtggacagg

SEQ ID NO: 231 - DNA, 20 bp, Artificial Sequence
  a primer sequence for PCR
cgcttttacggcaatcatct

SEQ ID NO: 232 - DNA, 20 bp, Artificial Sequence
  a primer sequence for PCR
tgttcgtcgtccttgtatgc

SEQ ID NO: 233 - DNA, 20 bp, Artificial Sequence
  a primer sequence for PCR
cagacgctgcataggatcag

SEQ ID NO: 234 - DNA, 20 bp, Artificial Sequence
  a primer sequence for PCR
ttactagcctctggggtgga

SEQ ID NO: 235 - DNA, 20 bp, Artificial Sequence
  a primer sequence for PCR
tctcttgcgctgtgttcact

SEQ ID NO: 236 - DNA, 19 bp, Artificial Sequence
  a primer sequence for PCR
atgcggcctttttcaacat

SEQ ID NO: 237 - DNA, 20 bp, Artificial Sequence
  a primer sequence for PCR
cgacgtaaggagctgtgagc

SEQ ID NO: 238 - DNA, 18 bp, Artificial Sequence
  a primer sequence for PCR
acctcgatcctgctgcaa

SEQ ID NO: 239 - DNA, 21 bp, Artificial Sequence
  a primer sequence for PCR
ggttgtcaggatttgtcagaa

SEQ ID NO: 240 - DNA, 20 bp, Artificial Sequence
  a primer sequence for PCR
ttacttcatccccggtggta

SEQ ID NO: 241 - DNA, 20 bp, Artificial Sequence
  a primer sequence for PCR
agagcatagcccggttgtta

SEQ ID NO: 242 - DNA, 20 bp, Artificial Sequence
  a primer sequence for PCR
cccaccttgatttgctcagt

SEQ ID NO: 243 - DNA, 20 bp, Artificial Sequence
  a primer sequence for PCR
agagcatagcccggttgtta

SEQ ID NO: 244 - DNA, 20 bp, Artificial Sequence
  a primer sequence for PCR
gccaccttgatttgctcagt

SEQ ID NO: 245 - DNA, 22 bp, Artificial Sequence
  a primer sequence for PCR
aagaacacagagattggtgtgg

SEQ ID NO: 246 - DNA, 20 bp, Artificial Sequence
  a primer sequence for PCR
ccaggaagacacttggaagg

SEQ ID NO: 247 - DNA, 20 bp, Artificial Sequence
  a primer sequence for PCR
agagcatagcccggttgtta

SEQ ID NO: 248 - DNA, 20 bp, Artificial Sequence
  a primer sequence for PCR
ccaccttcgatttgctcagt

SEQ ID NO: 249 - DNA, 20 bp, Artificial Sequence
  a primer sequence for PCR
cgcaagacttgaggaacaag

SEQ ID NO: 250 - DNA, 20 bp, Artificial Sequence
  a primer sequence for PCR
tccctctacgcagaagaacc

SEQ ID NO: 251 - DNA, 20 bp, Artificial Sequence
  a primer sequence for PCR
agagcatagcccggttgtta

SEQ ID NO: 252 - DNA, 20 bp, Artificial Sequence
  a primer sequence for PCR
ccaccttcgatttgctcagt

SEQ ID NO: 253 - DNA, 20 bp, Artificial Sequence
  a primer sequence for PCR
gaggcgctggtgtagagaat

SEQ ID NO: 254 - DNA, 20 bp, Artificial Sequence
  a primer sequence for PCR
tgtctcccgctttgtctctt

SEQ ID NO: 255 - DNA, 20 bp, Artificial Sequence
  a primer sequence for PCR
agacgtggagttcctcctga

SEQ ID NO: 256 - DNA, 20 bp, Artificial Sequence
  a primer sequence for PCR
caaacttcagttcgccatca

SEQ ID NO: 257 - DNA, 20 bp, Artificial Sequence
  a primer sequence for PCR
caactttcccacccaaagaa

SEQ ID NO: 258 - DNA, 20 bp, Artificial Sequence
  a primer sequence for PCR
aagcatgtatcggtggttcc

SEQ ID NO: 259 - DNA, 20 bp, Artificial Sequence
  a primer sequence for PCR
cgatacatgcttcgttttgg

SEQ ID NO: 260 - DNA, 18 bp, Artificial Sequence
  a primer sequence for PCR
cagcagccctcagcacac

SEQ ID NO: 261 - DNA, 20 bp, Artificial Sequence
  a primer sequence for PCR
ggcgtctatccgcaatacat

SEQ ID NO: 262 - DNA, 20 bp, Artificial Sequence
  a primer sequence for PCR
gagacaccgcatacccagat

SEQ ID NO: 263 - DNA, 406 bp, Penicillium coprobium PF1169
cccagcccaagacttgagtagactatatttattctctttgatatccatctcagcatcaagtttttgacgttgtattactatcctcgtttggaattctcctcccaggtcttgcttcattgcttatagcattctaccaaaaacgtcactgtcatggacgggtggtcagacatatcatcagcgcctgccggatacaaggatgttgtttggatagcagatcgggctctgctagcccaaggattgggatggtcaatcaactacctggccatgatataccaatcgcgcaaagaccgcacatacggcatggccattttgccactatgttgcaactttgcgtgggaattcgtctacactgtcatctatccttctcaaaatcccttcgagagagctgtcctcacaacatggatgg

SEQ ID NO: 264 - DNA, 20 bp, Artificial Sequence
  a primer sequence for PCR
cccagcccaagacttgagta

SEQ ID NO: 265 - DNA, 20 bp, Artificial Sequence
  a primer sequence for PCR
ccatccatgttgtgaggaca

SEQ ID NO: 266 - DNA, 39008 bp, Penicillium coprobium PF1169
gccagccaatgtctcgacgagactctcggtgtcaagctgcttgaggaggccattgtgaagatcgaagagcgtatcaggtcgcacggcggtagctgcaccgtgaagatggcacccaaggccgtcaccgagcaggacgatgcgatcctgcaggagcttatggagaagcgcgaacgtgagaacacccaggtcagcggagatgaggactctgaaagtgatgagggtgttcccgagtaagcgacgggctacaaattcgagtcgaggggcatacagcggtcaccagcgctaaaattcaaagctggtatcaccgctagaggggagttggtgaaagatggatagaaaaaacttgcacatatcggaaaaaaggctcgatgggccagtgtgctgatgggcaggattacagtcagaactcgcccaggtaagtcgcctggacttcggggtctggatatgacatattcacacctgtgtatgcggtattcccattgcggtcgaaatcctcgttcccggcatcaaatacactgggtccgcacagggtgcaagttctgatgcacataatgtttgatgcaaccgatacgttcaatgccagtcatgcttttagatgcaattatccctgtagaggccatgtagcaatgtatgtagcaatgtatgtagcaatgtatatagcaatgtatgtagcaatgtatgtaagatatcataacaatcgagctcatgaaatggcggggagagctgaagcttatctaccgccgccgatcattggtgccctcaaagccatcgagaacttccctttcggcacttctctttttccaccaactttcattctacgcgatatgggacattgggcaaagatctttaccgccgatcgtggcaggacccggcatcgggtcgaggtggaacgtcgtcggcgtacgtcattttccaaacatgcggaacactactgacaagccgcagtgctaccggcctatgcggccgatgagtctgacgcctcggatgcttcaaaggaaattgcaaaggttgctcttcggttgaaatatcaaattgagcaggttgtctcctgtgaagtggaggagaacgtcttgaccgacccaaacagccgtatcatcacggatgatgtggttgcgactgctaagcaggccggtggagatgaatacaaagcatgcattgtttattgtctcctggtttgtctgcgatggttcaaaatccaatcatccgtcgagctttgggattccgatctccatgagattcgagctgtggcttgcgaggtcatcgccaagcgcatgtaatgcccctttttcattccatgttctcggccatttcctgacccaaacagtatcgaatccgagcagaaccaagaatacgtgctaaaagacattttactcaagcgatactcaatcttcagtgaaggtgtggagactgatcccgccaatgtcattgaacgatcggtagatctccatgctttaaggatcatcagctgtgctgcgtaccagaagtgtatccagtatctctggagaggttggatctgccaggaagaaggcaacccaactaactttgtcgaatacagtgagaagtcaaaccccaattattgggttcatttccatcctgatcggatgcggactcctctgtatcagaatgtctgccaaattttgttttccttgatttaccttgcgacttataccgcagttatcaataccgtgaatcccaccggtgacctggatgtagctgaagccatactgtatgttatgactctcgcgttcatctgcgacgaggcggtcaaattctggaaggttggatggaattatctcgaattctggaatgcgttcaactcaacgctctactctatcctggcagtgtctcttgtcttgcgctttattgccttggcacactcatcatctacgcacgatgaaacaaggcaggcatacaatgaactcagctacaacttcctcgcctttgcgggccctatgttctggatgcggatgatgctatatcttgactcgttccgcttcttcggtgccatgttcgtggtccttcgagtgatgatgaaagaaagcttgatattctttgctcttctattcgtggttatggctggtttcttccagggcttcgtcggcatggcccaagtggatgctgatatccccatccaccgaaatattctccagggaatgatcaatagtatcatgcaaagccctgagtttgacacttttcaggaatttgcatttccctttggtatcatcctctattatgtgttcaacttcattgttatgactggtaagtctgtattacatttgtttggggtgtcgctaaacatttttagttctgttgaatattctcattgccttgtacaacagcgcatatgaagatatctctggcaatgccacggacgagttcatggccatcttcgcgcagaaaaccatgcagttcgtccgcgccccagatgaaaatgtcttcatcccacgtacgtgtttactcaattctgatatagcatacgtatgactaactttggtctgggtaatagccttcaatctcatcgagattctctgtttgatagctccattcgaatggtggctttcgcgggagacttacgccaaggtgaatgacattgtaatggccgtgatatattctccgctgcttgtcgttgcagcctgggttgagacccgtcaggcgcataagattcgatggaatcgccgtcatggcgaagaagacgatgactgcgctcaggaatgggagcatgtggccaaggaggtcaattttgatcttgacgatacctggaaacagcacgtaattgagtccacgccggatatcaaggttgatagttgtacatatgaactccgagagctgagggagcaggttaaaatgttgacggggatggtgaaggaattgactcaggagatggaaaagaaggcggatggagcaagctaggaagtcctgttgaattgtacagcaagaatactacactgagcatgggacatcgcaaaggtgatttgctactgcagtttcaccaatattacattgcgaaaactgtatattctcttaatgtctaatagcagcaatcagcccagtggcacggaggaaagtcaccgtcctgtaaggcaaatacttgtgcttcaaatgaattttgactatttttcatgcgataactggcaaagggcagggggagaaaaaatgatcattattcaacccaagcaaactgtccagaaagtgacatgcccactttgcaagtaaagaagatatgtgacaatctaacagtctcaggtagacattcgctcttcattaaaatccatgcgttgctcgccgtagcccaattcgaagcactgggcaacccacatcgagaccttaaaatcgggtgatcatcacacagcaacaggctcagcaagaatggaggcaatcgtctccctttgatgatccagctgtgagagcttcgctcgatggtgcttgccaatacctatccgaggaatgtcatccacaaatacaacacctccatctagggctttatagctggccagttggctttgaatcagacctgccacttgatcggccgtcgtctccggggacgtatcattgcggacgacataagctcgaggaacctcgctgctgccatctgggagcatgactccgatcacggctgcgtccttgatactcgggtccttgcgtaggatcccttcaatctctgcgggagcgacggagtatctaaatcaagcacgatatgttagtctatcatctgctgcatcggatgtcatatggagaggaaagaaagcgaaggatgtgaaggatgaagcctagaggactgggtaacttgccctcgaactttgatgagatctttggtccgtccgatgacatggtagtttccgtcttccacatggaacatgtctccagtccggaaccatccttgctcatctttggcatcagttcgtcctttgtatgctagaaggagtcccggtccacggacatacaactctccaggggagtctggtgtcccggcgacatcttcgcccgtgtcgggattgacaaagcgcagctcatatctgggcaaaagagtccctacactgccaaattgtggttgtatcccgtagcgattctggaaaaccactccaacctcagacatgccccacagatttcccgctatagcgtccggtgatagcaggctctggaattgctgcatagagtacccgtctatgggagcacccgaaataccgatatagcgcagagaagacaagctctcggctacattcaaggaggacctattgagaatgtggatcatggcaggaaccatgtacgtttccgtgatgtggtgctggcggatgccgtcgagcaaagcggtgatttcgaagcgcgggataatgtacagaggctggccgtaccgaatggggaagatgttgccccagaagtcgccaaaagaatggtacagtggcagtgccatcaaacgaacgacggggtatggcacttcatagtagacgctcagatggtgggaaatgatcgtgtggtgggttcgaattgcggctttggggagaccgctggtgccactggttaggaacatagccgccggcgtgatcttgctctcctcgctatcttcgaaacgaagccaatccaactcgccatactggagcagactctccaggcggataggttggtccactgtctgggtgtcgagatcctccgtctgctccgcctggccatgtgcaaattggactacactttcgatagacttctcatccatcagaaggacttggtttgaggacattccttgattattgcaaacttccaggactctggtcagcgcactcggagcagtaataatcaaccgaggctcggcgacacgaagcagatgagccacttcatgggggcgactagcgacatcaaaccccatatacactccgcccgcaccaacgatggcaaagaaaagagcagagtgtagaacctaggctgatgttagcagcatatcattgttatggggtagtgtgattacactgttctccagttgcacgagtacacaatcgcctcgttccacaccccgggctttgaggcccgcaatgagtgatcgcaccagccgtcggaattggatggcattgaaagcacgcgaagggttgcgggcatcaatatagatgggcttagattggtcaaaggcaggaccactaaaagcaaagctgactaggtctgtctcgtgctccatatcgatgcttatattgtacagttctcgtgtgctattgacatgcagaacttgatgcaggatttgtgctcactttaagtagtagtacatggaatgctcagacctcccatatcactttgatcgacactgcacgggacaagtatcatgcagaagactattgagaagaatgccacgccaccaattcgtattatactaatctagcctaagccaatacatgtaaagagtactatttaggacccacactgtcattgcagagctttgaagcagctgcatgcgctaattcacccacagatacgccactaagaatcaaaattaccccgatgtcgacgctcagctctttcgtaaaccattgactcagcccaatggcgataagcgagtcaatcccgagctcaggaatcagcgtgtcagcagagagcggtgcatcctcggccaagttcaagctggcccgaattttctccattagtggtctcacgactgcttcggctttttcttccaagcttgtcgctgcagtgacaagatctttggtcgaccgggtctcaatcaatgcaagtatttgatcttgagacgccgtggccgtataggagtagaatggccataacttcggtatcgggcactcgccatagccacacttcaaactctggtgtcggagtcccccgattaactcagcgttggaattggaatctgagcgcccgcagaggattgcctcggcgagtatctcgtcgacatcccgctgggatacagctaccgggccacaccaaagaggctgactgggagaaggactagaaatgccgtggatctcgcccagatgtactagacttgctggtctgccctgggctcgacggtggcggaccagcagggccattttctcggacatcgctgcagtcattgcctggtccgcatggcctaacactcctgcaatagatccgatgagcacccaaaagtccagagttggggtcttgtagagctcatctagctgctgcagccccttcaagaccggatgcagatggttccggagggaatctatcgtgagctgggacaaggaacagtcaggtagaggcggaggctgaattagaactcctcccactaccggggggaacgcatagggaatagtttgatgcaaactggtggcagaaatgccatcgatcaggtttctggcattgattagcttgtgcatttgaattcatgggtacgtaagtaacttacattttcgagaccgctatgcgcgttccccgtcgagaaacttcttccaaccaccacgcatctgagtcaagtctagagccagcaaggaggatccattttgccccgtgtgttgctagccaaagacagatagcatgggctagttcactgcctagacctacaagtatgtaggtttttttctctgacagctgcacctgggaaccagcagtcggtatctgggcgagcaccggagttgtagagtcccaatctaccacggcatcctgggagtcaaggattgggtactcggaaatcctgctgattggtaacgagtccacagaatttggtgggagcccctcgcggccggtgtacgccactaagcaggcggttaggaaagccttggctatgagtgacgaatcatccgcgttgatcggccctgtcgatgcagaggtaaggtagaaatcctgcaaatggattcgtgtcgcgttgtcaggcaagagcgatagcatgcgatcatagacaccctgtccacgacgatgtagaatcgctatggccgacacatctgagggaagtacctgagacaattgccgcgcagtgctatgctcgtgcaaaagcaacatcggtctttctttgtctgggttgcttttgctagtgctgaagataaccttaccatcccgccgggtcagcatttggtggaaaacagactgaaggaccccatcagcctcgtgcactacaagcgtgcctgattgtgggacttgttcgaccaggtatcctgccaacagagcagccgctgtggcgcggagataagactgctcgtgggcttccaacaccgtgtctggcactgaccacgcccaggagtctggaacgataacatgagatgcaatgtgggacgacagagcgatcattctcttgttgctcttaacgtccagccctatgaccagccgcagaaatatggcccctgcaacccgcacggctgctatactggaatgtcgaactcgcagatgaagggttgggccataacttgcggttatcggcggatcagccatcgaaagaaggcggaacccttcacaagtcttgtcggtcgtagcaggaaggatttgtacaactcccttgtcgaggtccacgcagtctgtcactttttgccgcctggctagatggcgcagcccagtagcatggtcgtgatattgccgaggaacacggaacatagagccgtcgtattgaatttcgggctcgatgttggtgagtccgcacgaatgaggattttcttgagctgcactagcttgaacaaagtgtccaagagctgtcgccaatatttccgttgtaactccaaccggatctgtgatatgtagcagttgtagcagagaagacgatcgttcagaagccaggaaggtggaaaggagacctttcaccagtccggcatcagggtggtcgaccttctcacaggtcaccactaacattcgcttactgactgtcatcagtctgcacaactcgctcaaggtagcgtttgtcagatctcgatcatcgacgaggtacagaacagtcaatttagataaatcgcgatcttcaatgagatctaggtctggcgcatgagcaaccttgacgaagtcatcctgtaccaattcaaaaagctctgaggtaagacaatctgcctcctcagcgtcaccgccaagcaacagcaggtctccgcggctctgtggttccgcaggactgtctggagtgcgacagagaaggagtgaaaagtccccaaggctttcactctcttgggacgcatcgaacgaatccaagccatagaatccgccgttagacagtagctcaacccagtccctcctggtagtaattggctcaccagagcagtagcctttcccggtctctgtgcatctcattggaggaccgaatagaagattcaaatatgtagtgctgggattcgttcgtactagtaggaccaagaatccaccaggcttgagcaagcgacgaacatgagccaccgcgacctcttgcagaaataccgcggctgtgatcagcaccatatcgtagaattgctcgcggcagccctgctcgacaggatcctcattgatgtccaacgttttgtgcgacacctcgccaggttgctcaaggtcttcctcaatcgcttgtaggccagaaacggagagtccagcataagtaaatgaccgataagtccgacccatcttcttcagtccagagtgaacatggcctccaaattggccgatctgaaggatattcatttgtggaaagcggaaacacgcctggctgacgacagatacgagctcgtcttctagatccaagacttgcaagtcctccttcagatattgactttcctcatcgatggccggccaggcctctatttgaagaccagaatcacgcagaacgcgaggtagccgctggcctaccgcagcaatggccgtgagaccagggtcattcaagagtgacgggctcactccagctgtgaagtcctcaatcttctggtccaagcactcggattccccgacagggtctggctcttggctagcatttgcaatgcattggttcatccatgcaagcagacgagcaccatcgaaatccaatccgcttcgctccaagtccgtcagtccattgcgagcctgcttgagatacagtagcgcaagctcctctcggagagagtgtagctgtagcatggtagctggcaacttccgtgatcccttcttcagagtgggctcaagcggtccccacgcggtctgggaaagaacctgcaagttgtttggtgcagttccagatggctggcatatgagagaaacaccttcgagctggacagccttttccccattcatggtgaagatgtcaatatcaccgcgaattcgatctccattaacgcaggtcagatagcttgctaccgtcaattccttgccttgccaatctgaagcacataacaccggatttatccaggtactgtcaacatttctcgataagaatggtcccgtcagcagcgtctcttcaagcccaccaattgcagcaatcattgtttgaacaccaaggtccaaaatagccgggtgaagagccatgggctcatccgaatcatttgagggaacgggcacactcccagtggctagatcacgccttttgcggagtcccgtcaaggtagagaatgggccagtacagtggtagtcagcgcggcgcaggctgtcatagaattcagtgctgtccacgggctccaaggcctgaggtagctgtccctgtgggggtaggagagcacggtcagaatcccctggatgcatgatcatcttggctgttgcacactgaacgagctctccggatacaacagcttcgcagcagaaccaagcagtaatggctccatcatgcgagtgaatactacccacggtgacaagcacttcagtgccgatgggatcattctgaatcgggagctgagtgtggatggtcaagtccttgacattcaacaagcgtaggccttgtgtctgtgccattatcatacctgcctccagtgccatcgatatgtatcctgtctcagggaagacagatcccgaatcggcacgacggtcggccaaccagggcagctcctctggtcgtagatagtttcgccaacggaacttttctgctccggtctctggactgagagaaccgagaagtgcgttaggagatgtagcacgatggttatggttcgaagacattcgcgactgtgtccagtatgtctgagtatggtcgaaggggtagaatggtagcgattctaccaacacaggccaatgatttggatcaaagagtgagacatagtctgtgaggcggacgacatttgggccgaggtgtgcccaggaagatcctagggccgttgcccatgtatcgaggccgggctttcctcgctcagcaagagcaaggtaaggaattgccgagtgggccgagtgcattttggagagggtctgtaggacaggccctctcagtgtcggatggggcccgatctcaatgatgagatctggtggcccagcgtctcgtgccgcggcctctagggcttgggaaaactgaacaggacgcagcatattctcaacccagtactcccctgtcaattcctgctggtcatacccagtcatgacctcccctgggtagacactcgagtaccagcgcgaggcagaagctgacagggcgacaggatacgctttcattgcgtcacgatatggatctgcacaaggcttcatatgcggagagtgatatgcggtgtccactcgaagcatacgcggagtgaggcccaggctcttcagcagccactccagctcccgcaggcactctgcgtcgccggataacgtgacgctggatggcgagttggcggcagcaacgcttatacgtccggagtaggcttctaaagcacagatattctgcgcctgctgccatgtcaaattcacggccatcatccgacctgtcggatcgcgtgacttatcaatggtcatccccctaaggtacgcgatgcggattgcatccgaggccgtcagcacacccgcagcataggctgctacaatctcgccggaagagtgaccgaccacaatggtaagctcaatccctaccgcacggagcatgttgacttgcatgatttgcaacgctgtccgtagggggagagaaaggaggccctcgtttacgcgcgaggacgatgccggctgtgacaactcgtcgagaagagaaaactgtggacgaaggtctagtggaagctcatccaaagcttcctccagattcataatccattttcgaattgagggacttgcctcaatcagatcaagtcccatttgtggccattggactccttggcccgtgaagatgcccatgacgcgtctgggccgagtgttggatctggagacgacagaggctggtttacccgtgacccttcgacttatttctgtattgatctggtctttcaactcttgtattgagtgtgccattagcgtcaaccggtggcgatgagtggaacgtcgatcccacaaagagagcgccagaccaacgagactgactgttgcgtgttcctggaggaatgttgcgtatgattccatcacacaagtgagggtccgctcagacgcagcagaaaagacaaagggcagactggagggtatgttgttggacggactgagctccgagcgagtgtagctttctaggacgacatgcacattggcacccccgaatccaaaggagttcaccgaggctcgacgaggacagccatctgggactgcaggccacgggatgcattctgtggggacagaaagcttggcggcaaacggtttaatttttggattgagatgctgcatcaggagattaggagcaatcatcccgtgttgcagcgagagggatgccttgatcaatcccgctagtccagcagtggcctctgtatgtccaattattgtcttaattgacccaacatacagccgatcggtcgaatctggaacggattcgggcccaaagaagcttgagttgattgcggctgcttcctgcggatctccggcctgggttcccgtgccatgagcctcgaagtactggcaccgatcttccgggttgttttgaggagagagccccgcgcgtgcataggttgcgaggatcaatgattgttgtgcctttggattaggcatcgtgatccccatagttcgcccatccgagtttgcccctgaggcacggatcacgcattcgataggatctccatcattaatcgcgtcctgcagacgttttagtacaaccgaagccacaccctcgccacgtccgtagccgtcggccttgctgtcccacattctactccggccggtaggggacagcatccgtgttttagaatccgcaatataggcattgggagacaggatcaggttgcttcctactgccactgccatggaacagtcatcgttctgcagagcctcgactcccagatgaacagccaagagactcgaagaacatccggtgtcaacggccatagaaggaccttgccagtcaaagtagtaagagatacgattggccatgattgacggtgagtttcccgtaaccacatacgcggggaacgcctgaggatccatggcctggatttgattgtaatcgttgcgaagtgtaccgcagaacaccccggtctttgagcgctgcagcgcatccatccgtaacccggccgcatcgagcgattcgtacacggtctctaggagcaatcgctgttgtggatccattgctaccgcttcagttggcgagatattgaagaaggccgcatcaaaggctttgatgtcctcgtccaagaagtatgactctttgacgtttgttgtgccatggtggtctccatctggatgataaaaggcatctatattgaatctgtcggccggaactttgcgcgcgatatcccgagggctttgaagaagctcccacagtttcgaaggagaggaagcgccaccgggaaagcggcatcctgtaccaataatagcaacaggctctgttgctttcattgtgagattataagagaggtgtaaaacctgagatcaaaataatttgcagttgggtggctgtagctctactgagagtacgttcatagatataagcaatgcagtgttgccttacttacttccacgatcttgtcagcatatctatcgaacgaatagcaaaactggacctatagagcaatttccggccatcgatagatcattggatagctgtcctatttgggaagtatgatctacaatttatgcagccacaaactatacaaagtggtccatcgccagatttggcgatgagcagcggtgtggaatagtgactttgatgaacatgtcaggtcctgcatctacatgtgcaggtgtccaaggatgctccttgcgcgaagaagtggagtagggacattcagctacctccttatcttttcccttcttttaatgctcactctgtgcataataatagtggcgaatatcgaagcatcgaaatccaacgacattgagacaacatggataacatggacaacatgaacaacacacctttaggtttcaactgggcctgggcagtcatcatctctttcctgggtctgctgactttttcctttgtctcgccacacctctttccttcaagattgacggtgattaatggtggaagagcctgggatatctttcgtaccaaggccaaaaagcgatttcgctcggacgcagcacgtcttataaagaacggcttcgaggaggtgagtatggaaaaactgcatcatttaggataaagtgctaaacgttccttcttactccagtctcctgatgcctttcgcattatcacggataacggtcctttgctggtcttgtcacctcaatacgctcgtgaggttcgcagcgatgatagactcagccttgaccatttcattgcctcggtttgtcttgcttcatgtccaacgtttttctagttggcgtcgctaagcttctactgtttaggaatttcaccccaacatcccaggtttcgagccgttcaaattgatcttggatccaaagaacccgttgaacacgatcctcaagtccaatctcacacaagcactgggtactgacatcgtcctctccgctcttatgcagcccattacatagctaacattgtttacctggatagcttatctgacagaggacttgtctgcggaggtaacagaggcactatctgcaacctgtaccgatgaccctggtaagctataaaacatggttttccaaaggttctggtatcaatactaactttctttcttctcttaatcaaagagtggcacgaggtcagcgttagtcaaacggctctcaaaattatcgcacaaatggcgtccaaagccttcattggacaagaaagatgccgggatgccaagtggcataacattatcatcacgtacacgcacaacgtctatggagcagcacaggcactccacttttggcccagtttcctacgacccatagtggcacagtttttgccagcatgccgaactttgcaggctcagattgctgaagcgcgagagatcttggagccattggtagcccagagacgagccgagagagccacccgagccgctcaggagaagcctcatccgtctggtggggatatcattgactggctggaacagttttatggggaccaaccgtatgatcccgtggccgcacagctactgctctcatttgctgctatccatggaacttccaatctcctggcgcaagcgctcatagatctctgtggccaaccggagctagtacaggatctccgggaagaagctgtgtccgtgctgggtaaagagggatggaccagggccgccttgtaccaactcaaactaatggacagcgccctgaaagaaagccagcggttggcgccaaacagattgtgtgagtgggcccttcctcttgccccccaatttgaccattcaactggccattagagactaattcaggtgtgcttttacagtatcgatgggacgcattgcgcaaggcgatatggacctgtctgatggtctccgtatccaccggggcacgaccctcatggtgtctgcccacaacatgtgggatcctgaaatctaccctgatccccgaaaatacgatggctaccgattccataagttgcgacaaacatcagggcaagagggccagcaccaactcgtatcctcgacgccggatcacatgggattcggatacggaaagcatgcttgcccgggacggtttttcgccgcagcccagatcaaagttgcattgtgcaatatcctcctcaagtatgatattgaatacaggggtggcaagtccccaggtgtgtggggtcagggcatacatctgtttcccgatccgacgtctaggatccacgtccgtcgtcggaaagaggagattaacttgtgatactattgtctaactatgcggatgtggttgaatgcaaggactctctctctctctctgtctgattgatatttgagttttctatggtgatcgagcaagatttttgcaatgtggagcccatgcatgctcatgaggcctattgggccgatctcttcgagatcgtgatcgagagcaaatttgagaacctcagaccttgtttatttgaaagtagcagatgaacaatagaattgtttttacttttggaatggttccacaataatcctagtctagatttaagataccaatattgaagtgttatgtttgcatgtatcttcagctgctccacccgcgtggagtgattattagcttattagcgccttctcattaatacgccctccagttccagcctctcaaaagtaatatgctggaatgatagaggtaattggctaatggcctcaaggcaaccctgcagatagtgaagcaaaagcaataaatattcaatattcacacataatttgacatacggagtactccgtactccgtttaagatcgggcatagtattggatgatgttagaatatatcttggcaaggtgacatatacaatgtactccgtatgttgtacagtgtcaatggctttgtggagctgaagatgcggtgatttcttttcctgatgcatcatcaagtccggaaaattgatgaaaatctacgagtacctcgagggatgaacttccctgcacagatcatgacatacatataaactattgatccacttgcattagcgggagtctagcaagagcaagtctatgtattccctacatggtcgaggaggtaagttcgggctgaaaaatacgatgcagcatacactacccttacaactagctgtttaatcagaaaaagcaaatagaaattagggcacaatttactctttactgccaaccccccgtcgtaacccttgctgctagcattgattggctgtcagtcgtacaacgaagaaacgacactgtctgtgattatattctattccatcacaaacgtagcccggagtgcccttcccagagtccttgtcttgtacaccgtgcttgtcttagcattttcattatgatcgagctcaaagatgcttcgatgggggctgtattgctgacatgcgtccttgtgcttgcaggcctatatctcattcgattgacgttatcaagcgaccaattggacaagtttcctagcatcaatcctcggaagccctgggaaatcgtcaatgtcttcgcccaaagaagatttcaacaggatggccctaggtatctggaagctgggtatgcaaaggtgtgttccataagcaactgctccaaaaggcgaataaggctgaaagttactacagtcccccatctttagcgtggtcaccgacctggggccaaaattagtggtttcgggtgcattcatcgaggaattcaaggatgaaaagctgttggaccattatcggtcaatgatcgaggtttgtacgacgttagtgattatgaaagagcaagcgcttacttgtgcaaggacttcatggcagaggtacctggttttgagtcgatgttcctggggaatctacacaatacggtacttcgcgatgtgatttctgtcatcactcgcgaactaggtaaatattctttccttttgactgtccggttatccgctgagttctaattttatagaacaactgctagcacctctctcggatgaagtatcagcggctctggtagatacttggacggactcaccaggtgggtcaaagcacacttcccaatagaaatcaggaggaaataaaaactaatatcaatatagactggcatgaggtagcactgcttccaagcatgctgggcttgatcgcaaaggtttcatctctcgtcttcgtgggtgaaccgttgtgccgccacccagtctggttggagacagtgatcaacttcaccctcattcgacacaacgcaatcttagccctccaccagtgccctgctgtacttcggcccgtccttcactgggttcttccaccatgccagaaactccgacgagagatcagaactgcacggacactgatcgactctgctctggaaaaatcaagaaagaatccgcagaccgagaaattttccagcgttgcctgggttgatgcttttgccaaaggcaacaagtataatgcagccatggtgcagttaagactggcaaatgcgtccatccactccagcgccgatctcctggtcaagattcttatcaatctatgcgagcagccagaattgattcgggacctccgggacgagattatctctgttcttggggagaatggatggcgatcctcgacactgaaccaattaaagctccttgatagtgttctgaaggagagccagcggttgcatccagtcacaaccggtatgcatcgtcggctgttcaaactgcgtgcccagtgcatatgctgaccatttactttaggagcattttcgcgctttactcggcaagatatcaagttgaccaatggcactgagattccttcaggaacacccattatggtcactaatgatgtcgccggggatgccagtatctatgatgatcccgatgtcttcgatgggtatcggtacttcagaatgcgtgaaggagccgataaggcccgggcaccattcacaacgacgggccaaaatcaccttgggtttgggtacgggaagtatgcttgtcctggtcgattctttgctgctaccgagattaagatagcgctctgccatatgttgttgaagtatgaatggaggctagtaaaggacaggccgcatgggatagttacaagcgggttcgcagcattccgtgacccacgagcaagcatagaagtccgcagacgcgcggtggcgggagaagagctcgaggtattgactggaaagaagtgatctagggaaaattacgaactcatagtatgagcaaccatacccaaaacaaagagacttaccaaccccatcatcaaggtagactggggattttgactatgtcgatgtaaatcggtcaacagccttattaggatatataaattatacgcttctcaggctttaaagcatcacccagcacgataatttctctggattattgcaaaaccaagaaattctctgatccacagctgtatactccgtactccgttcatcatcttacagtcatgcagagggtgaaaggggtcagtgtgtgacggtatttcggtatctcgcctcgtaatttgacagatccagcgttaaacccagcccaagacttgagtagactatatttattctctttgatatccatctcagcatcaagtttttgacgttgtattactatcctcgtttggaattctcctcccaggtcttgcttcattgcttatagcattctaccaaaaacgtcactgtcatggacgggtggtcagacatatcatcagcgcctgccggatacaaggatgttgtttggatagcagatcgggctctgctagcccaaggattgggatggtcaatcaactacctggccatgatataccaatcgcgcaaagaccgcacatacggcatggccattttgccactatgttgcaactttgcgtgggaattcgtctacactgtcatctatccttctcaaaatcccttcgagagagctgtcctcacaacatggatggtcctgaacctctacctcatgtacactaccatcaaattcgctcccaacgaatggcagcacgccccgctcgtccagcgaattcttccagtgatattccctgtggcaatcgcggcatttacggcggggcatctcgccttggctgcgacagtgggagtggccaaggcagtcaactggagcgcctttctgtgctttgagctattgactgccggtgccgtgtgccagctcatgagtcggggatctagcagaggggcgtcgtatacaatctggtatgttctttttgccttgtggatcttgcttgggtttattggctaatgtgaattgtggttggcagggtctcaagatttctgggctcgtatatcggtagtatctttatgcatgttcgagagacccactggccgcaggagtttgactggatcagctaccctttcgtggcgtggcatggcatcatgtgcttctcgctggatatttcttatgtgggcttactgtggtacattcgtcggcaggagcgccagggccaattgaagaaagctatgtgatcgacaggaccatgcatgatggaggtccgcactaacctcaactgtactttgtacaggtctgagtgctatatgacgatagtcacaaaacagagttggaggttatttgcgcacattgactaaaaatgggagagctgatggatatatgcaagggggatcaggtctcgatctgatcgtgccgatcgacaagaacaatgctttgtctgggcgggtccaattgtctagcctagaagtctaaatttcaattttcttcggactttttacatagtaactactgcctaggactcgggatatgaagtataatggcgagaaatggctggctgcaggggacatacaggtgataatttgccctcgatctggcagctagttacgtcaatatcttgttagtaaacaccagttgtagatctttgcgtatatatgaaactcaaaagcatttgtgtctactccgtaattaccttcccaacccctccagtgccattgaaaccatgaaggtcatcattgtcggagggtccatcgcgggtctcgccctcgcccattgcttggacaaggccaacattgactatgtcattctagaaaagaagaaagaaattgccccccaggaaggtgcttccattggtatcatgcctaatggtggtcggatcctggaacagcttgggttatacgaccagatcgaggagctgatcgagcctttggtgagggcgcatgtaacttaccccgacggcttcaactatacaagtcgataccctgcactcatacagcagcggtgcgtcaatataagctttctactttctgatttgaaactaatgcgagaggtcttaggtttggctatccacttgcattcttggatcgacagaagttactgcaaattctggcaactcagccggtccaatccagccgagtgaaactagaccacaaggttgagagcattgaggtctccccatgtggcgtcacggtgataacaagcaacggacacacctatcagggcgatcttgtcgtcggggctgatggagtgcatagtcgggtacgagcggagatgtggcgactggcagatgcctcgcaggggaacgtatgtggaaatggagacaaaggtaacattattcctactgttttgtcctatcctcgcttttttttttcttggccaagtgttttgactttgagctggaaagctaatatattgatttatagcatttacgatcaactatgcctgcatctttggaatttcgtcacacgtcgatcaattggaccctggcgagcaaataacctgttacaatgatgggtggagtatccttagtgtgatcggacagaatggcaggatctactggttcctctttatcaagctggaaaaagaattcgtttatgatggatcacacaaaacccagctccactttagccgtgaagacgcccgagctcattgcgagaggctggcgcaggagcctctctggaaagatgtgacatttggtcaggtctgggctcgatgtgaggtctttcaaatgacacccttggaagaaggggtgcttggcaaatggcactggagaaacattatctgcatcggagacagcatgcataaggtcagcagctcattatcactcctggcttactgacttttgtaattaattgacattctcatgcagttcgcaccgcatattggacagggtgctaattgcgctatcgaggatgcagctcagctcagcaatagtttgcacacttggctgagcggatctggaaaggagcatcaactaaaaaccgatgatttgacagagattctggctcaatttgcacaaactcgcctccagaggctaggtccgacggccatggccgctcgatctgctatgcgtctgcatgcgcgggaagggctcaaaaactggatactgggacgctacttcttgccctacgctggtgacaagccggccgactgggcctcccgaggaatcgcaggtgggaatactttggacttcgtagagcctcccacgcgggctggtcctggctggattcagttcagccagtcgggtaaaaggacttcgtttcccatggcagtggcaggtctgtgcctagtgagcattgtggcccgaatcatgtatttgaaattagttgcatagagaggcccaccatatctggagtacttcatacagagtgttttatgggacaatataaactttagggcaatttagcgctttgatatagatcatctgcatactagtaaggcaaccctgaaggtgatcgacacgatctgcaaaaatcaatatcgtgcttcgttacggagtattgttttctacatgtcatagtgcgcgctgccccagtggggctatgcagaaagtgatttcgatgtattgctacttacagtgatgtggtccagcatgtcagccattgctctagtgcgtgcgtgtactgaccacatcgcggccattgccatttatctagggtcctgtcgcctcaaaagcttgtggtcacaaatcgtttgatccttcgagatcatactgaatttttgttcaatctgtcatcatggctggctctcagtctacggcgcagttggctcgccttctcattgatatctcccgatttgacaaatacaactgtttatttgctatattccctggaggtacggagtagtgcagaccacttcaacattataccaccgcgctcacaatttcatatagtctggtctatcttccttgcagcagcctcacgacacgctgatggcgaccccgtccctctggactttgtattgggccgcgcaggactggccttcatgtacacgtatatgctgagcggcgcaggaatggtatggaacgactggatcgaccgcgatatcgatgcccaggtggcccgtaccaagaatcggcccctcgcctccggtcggctttccaccagagctgccctcatttggatgcttgtccagtacgcagcctcggtctggctgatggaccgcatggtgagcgggcaggatgtgtacgtcttttttcctcctcgtaccccaaacaattattctgttgattgaaaactgaccctaatcattctccagatggacatacatgcttcctctcacaaccgggattatcttgtatcccttcggcaagcgaccgacaagtcgcaagctgggcgtctatccgcaatacatcctcggtgcaagcagcgcccttactatcctcccagcctgggcctccgtctacacaggccgtatatctttgaaggatctgggtatgcggtgtctcccgctttgtctcttcctgtttctgtggaccatctacttcaacaccgcctacagctatcaggatattaaggatgactgtaagctgaatgtgaattcgtcgtacgtcctcgcggggagccatgtgcgtggaatgcttctgttacaggctattgctgtggtgctggtgatcccctggattctctacaccagcgcctccacttggctctgggtctcatggctgggggtatggacggcatctctcggcgagcagctttatctctttgatgtgaaggatccgagtagcggtggaaaggttcatcggcggaatttcgcactggggatttggaatgtgctggcctgctttgttgagctgctatatgcttcaggctctctgtgaatgatgttaatacgatgtggtccggatgagacttggggagtagagtctgagaggcttaaaatgggtaaatggtgcgatgttggcacagtgtgaactattcataaatctttgctacgaagttgggcttcacctttcaattgagaagttgttactggaatttttcgacactcaaaattcgaagagacttgtattattagagggatatagcctatgtcttccaattggtgtagaatcccaactacgagaccgcttcagaacgttggagcacaaggatagaaagttcacctattcgaaattctctactgtcgtacatatgctatgtacatgttactcctttgcttgcgcacctatagcccagcaaaacaagggatcctttgctaacaggagctgatcatcacggttcagagtcagatgcaaatcccacggctccgtactcgccacatcatcctgaccctttggaaggataaagcacatcccccctaagacaggcaaatgtagttggaaccctcgaggttgcgctccaaggctctccccaaagtccagtccgaagatttcaaaattcctaaagctgctcagagggataggaactccccgaaatccgatatccgcccagtcaggctgcgagtggagatgggatagagcgtcttgaatatattctgcgtcaaccgccaaaagactctggcgtatacgagctgcaatttgtgtgagatcctccagacactcctggcgcaattccaccgaaggatctgtgccatcaactagggcctcgtttctcccagcttgaattggcgtatatgtcaatagcaccatgtttcccagatagtcatcaaaggctggagttttgaaattcccacgcatatccaccgcgattgacagttcggtagatttaccagccaattgtcccgcttgccgaagtatcatggccaaaagggcgctcacgatgtcgttactggacaggaaccctggactcggcctaccatcagcctggaaagacgtttgccctttgatcaacgtattgcaagcctccttcaaatactcgatcttaggaccggggattttcagtcgccaggtgacaagctcggtggctctcgcccggacgaagcccgaccaattctttgcaagtagtgctgcccagtctccgaggccacagtagtgcttgctaaaatccatcctggaaagaccggagctgctttctgggacaagacgctcaatctccgatcgtaactgccgatctggcgacacacttgcagaagacatcgccgtcgggtctctgcagcaatcggctagaaggcccaagactcgcgcagcgcctgcaccatccattgcggaatgatgaaacgtcatggcgagaatgatcccatcgcgcatgacatttgcttgaaatcgtaggatcggccttcgtggcaacgaaatatccatgtcgataggcaatggcgccagccgacttatgatttcctgctcctcagtgcccgttaggaggcattttgattggatttccttgaatgactcggcctggtagtgccgtatccggagtatagggaactggacaagcgactctgaggcttctggttcgatttgccaggtgtacttcgtttggctggactctgtccgccgagtcacgtcccctgcgaggaaggggtgtaccttcaatagcagctcgatgccattctcgagaacaccaatgctcttctcaggttgcgtggtctggaaaaacagcagaaaggtgacgttcattccgaggggattgtggtcgagagaagataaagggtaagcagagcggtcgccagttcttcgggcatcgcacattccatcttcacatagaccgtggagtctcacaggtccctctttgacctgatctctttgactgactgggagacatacttcctgggtgctcatgatttctgggtgttatcctattgagttgagttgtgtcttgatctttttttttattttttttggatttctgaccttgtttcgcttatattggactttgcttttctttgtatattgtattgcattaccgtacaacaaagcatgggattctctgtgttctgcatgattgtggagcgtattttcctcgatttggtatacaatcaggtcgatccctggcggattccggatctgatgcatgtatacaggtcatatatctgctttcctcggtatttttgagctgaatatcactatatatgctttggagacgatcaatcgcaagagagggttagtgattaaatcagttagtctcatccatagtgggcattagagccaataaaagatggtttccaccttgagatgtgatcgccacaagaagattttgtaaatagtatgtattttccaggccctgatttctatctgcatatttgtcagcttgatctacggagtacatcttactgcttttagatactgacagcagcaaaactccgcgttgaaggacgagctttgacacaaggtcaggcacttctctagtacacaaatcctaatcatccgacgacatactactccgtatgctgtacatagagatccatgtccaattcttgagtctgcccctctttgatccacagtccagctcagccaggcgcaatctgcatgcattggcatggaagctaggagctgacattggctggaactacgccatctggggcacaatgcaagctaggcaactgaccatgtactgggtcagttttgattgagtatgctatacggaagaaagcgactagtactccgtaggtttgtgtactacctgcaagtggaaagagatacctagataggtgacattagtgtccgaaccaatgaccaatggcccttatgcacccatatcccttacatctttcagaaagagaaaagccacaagtatatcatgtactccgtactccgtacaacggaattacttgatctctatattaccttcttcctgaagaccgtttctcgctattgtcagttacacacacaatggattccctattgacgagcccgttatggctcaaaattgcacatgagctagcactttacctctcttttattgtgccaaccgcctttctcatcataacaactcaaaaatcatccattattcgatgggcctggacaccatgtctgctttatatcctgtaccaattctctcttcgggtaccctctctgtcgacaagtcaattcttgaagggcgttgcagcgggtcaagcaaccgtggctgctttgcaatgccttaatcttcttctgatcacgaagctggaccaaacggatctgctacgggcaaatctatacagtccgtctgcaggactgctttctcgccttgctcaatcctgcgcattgctggtcaacttccgcggaatcggcacaatctgggaggttagaaacattccccagcacgcagcgtttgtccaaccaaaaggcaaggatcaatcaatgagccggaagcggtttgtcttgcgggaaattgcaatcattgtatggcagtacctgctccttgatttcatttacgagtcaaccaagggcacgtcagccgaggatttgatgcgtctctttggccctggtatggaaatcaagtatctcgatgcaacgttcgaacaatggatggggcgcctctccgtgggaatattctcttggcttgtaccttcccgagtctgtcttaatatcacttcccgcctgtactttctcatcttggtagtattgggcatttcttcgcccgagtcttgtcgaccgggcttcggcagagtgcgggatgtatgcaccatccgtggagtctgggggtaagtgaactattccgactgctttcattcattcactaacgccaccacagcaagttctggcatcaatcctttcgttggccactcacctctgtcggaaactatatcgcaagagacgtcctcggacttgctcatccctctcttttggaacgctacaccaatatcttctttacctttttcacatccggcgtattgcaccttgtctgtgatgctattctcggcgtcccgccatctgcgtccggcgccatgcagttcttctgctcgtttccgcttgctattatgattgaggatggggttcaagaaatctggcggagagcgacgggccaaaccaaggacagtgatcgtgcagtaccgttctggcagaggctcgtgggatatctttgggtggctgtctggatgtgtgtcacatctccgttctacttgtacccagctgcgcggcaacatgcggagaagaactggatagtgccattcagtatagtggaagaaattggccttggaactgcgcaaaagattttgctgggttatggcttgtttgtgtactgggcggttggtggggagatttaaattcatgtgtcgggattgttcatcgtggtcaacactgtttagattgtgatatatattttcaccgaacaccccagaaacaaaagatttaagccccaattaactaccttgaagggctcatgagatttgatcaatgtagcaaccgtcagtatcctaggtcgtgattcccccagccagagcgagataattttccagacatcatcttatctacatgcaaccaaaaactccctggcatatattaacagagcaaaactagaggagcaaaaaagaaatctcaggtttggtttttaggaatagccgaacgcgggggtcgaacccgcagccttaagattaagagtcttacgctctaccgattgagctagcccggccgggctgttgaagagagttgccatatagcgctacataatcctaaagcggtcagggcctggggggcgaacacgctgacataatgctagcgcgtcgagcggcgaatcctctggaaccaaaattgttaggtggaaggtggcttcatctacgaatctgggtgtttcctcgattggatcttatcattgcttccctgattcgtatgagtctttaattttctggttgcttgactctgaccgcggtcactagattgcccaccatgtgcgttactagaacctttccccgattctttgctgcagctaacactatacagggcaaagctcgtggacgaccatcagatccatactgcctcgttgcataacccgattccttggcaattgcatacatacgtctggcctttcctgatcatctggcccgtgttctttgccttttacctctctcccgagcgctatgatacctacattcagggacaggagtggacctttgtgtttgcggggtctatcatcacagtccagtcgctcttctggctgatgaccaagtggaacatcgatattaacaccctattcacaactactcgatccaaatccatcgacactgcccggcttatcaaagtggttccgatcaccaatgccggctctgccgagatctgtaacctgattagagagcacattggcccgaagaagaccctttcgttcctcttccagaagcgccgcttcctcttttaccccgagactcgctccttcgcacccctttcttacgccctcgacgccgagccgaagccggccctcaagactttccagcagagcgagggcttcacgtcgaaggccgagattgagcgcgtccaaaaccactatggtgacaataccttcgatattcccgttcccggtttcattgagctcttccaggagcatgccgtcgcgccgttcttcgtcttccagatcttctgtgttggattgtggatgttggatgaatactggtactactcgctcttcaccctcttcatgctcgtgatgtttgagagtaccgttgtgtggcagcgccagaggacattgagcgagttccgtgggatgagcatcaagccttacgatgtctgggtataccgtgaacggaaatggcaggagatcaccagtgataagcttcttcccggtgatctcatgtcggtgaaccgcaccaaggaggacagcggtgttgcttgtgatattcttctggttgaaggcagtgtcattgtcaacgaggctatgctttctggcgagagcacccctcttctgaaagactctatccagctccgtcctggcgatgacttgattgagccagatggattggataagctctcgtttgtgcatggaggtaccaaagtcctccaggttactcaccctaatctgactggcgacgcgggcttgaagaacttggccagcaacgttaccatgcctccagacaatggtgccttgggtgtggttgtgaagaccggtttcgaaaccagccagggtagcctcgtccgtactatgatctactcgactgaacgtgtctctgccaacaatgttgaagctctgctgttcattctcttccttttgattttcgccattgccgcttcgtggtacgtgtggcaagaaggtgtgattcgggatcgcaaacgctccaagcttctgctcgactgcgtccttattatcaccagtgttgttcctcccgaattgcctatggaactcagcttggccgtcaacactagtcttgctgctctgagcaagtatgccattttctgcactgagccattccgtatcccctttgctggtcgtgttgatatcgcttgcttcgataagactggtaccctgaccggagaggatcttgtcgttgatggtattgctggactcactttgggtgaggctggttcaaaggtcgaagctgatggtgctcacaccgagttggccaattcttctgctgctggacccgacaccactctcgttctcgccagtgctcatgccttggtgaaattggatgagggtgaagtcgtcggtgaccccatggagaaggctactttggaatggcttggctggactctgggcaagaacgacactttgtcttccaagggcaacgctcccgttgtttctggtcgcagcgttgagtctgttcaaatcaagagaagattccagttctcctcggccctgaagcgtcagagcactatcgcgaccattacgaccaatgaccgcaatgcttccaagaagaccaagtctacttttgtgggtgtcaagggtgcccccgagaccatcaacactatgctggtcaacacacctcccaactacgaggagacctacaagcacttcacccgtaacggtgctcgtgtgcttgctcttgcttacaagtacctttcttcggagaccgagctttcccagagccgtgtgaacaattatgtccgcgaagagatcgaatccgaactgatttttgccggtttccttgtcctgcagtgcccgctgaaggacgatgccatcaagtctgtccaaatgttaaatgaaagcagtcaccgtgttgtcatgatcaccggtgataacccattgactgctgtccacgtcgcacgcaaggttgaaattgttgaccgtgaggttctcattcttgatgcccccgaacatgacaactctggaaccaagattgtctggcgtaccattgacgataagctcaaccttgaagtcgaccccactaagcctcttgatcctgaaatcttgaagactaaggatatttgtatcactggatatgccttggcaaagttcaagggccagaaggctctccctgatctgctccgtcacacctgggtttacgctcgtgtctctcccaagcagaaggaagagattctccttggtcttaaagatgctggatacaccactctgatgtgcggtgatggaaccaacgatgttggtgctctgaagcaggcccacgtcggtgtcgcgcttctgaacggctcgcaagaggatctcaccaagatcgctgaacactaccggaacactaagatgaaggagctgtacgagaagcaggtcagcatgatgcaaagatttaaccagcccgcccctccagtacctgttctgatcgctcacctgtatccccccggccctaccaacccacactacgagaaagcgatggagagagagtcgcagcgcaagggtgctgcgatcaccgctcccggcagcactcccgaagctattccgactatcacatcccctggcgcacaggccctgcagcaatcgaacttgaacccccagcagcagaaaaagcagcaggcccaggcagctgcagctggccttgcagacaagctcacatcgtctatgatggaacaggagctggatgacagcgagccccccactatcaagctgggtgatgcatccgtcgctgctcccttcactagcaagttggccaacgttattgctatcccgaatattatccgtcaaggtcgttgcaccctggtcgcgactattcagatgtataaaatcctcgctttgaactgcttgatcagtgcctacagtcttagtgtcatctacctggatggtatcaagtttggtgatggacaggtcactatcagcggtatgctgatgagtgtctgcttcctttcaatttcccgcgccaaggtatgtcgtatttcccatgtcgaccaaatgatttgctaatatgttactgtgtgaagtctgtcgagggtctgtccaaggaacgcccgcaacccaatattttcaacgtctacatcattggatctgttcttggacagtttgccatccacattgcgactctgatctacctttccaactatgtctataagcacgagccgtacgtgatgaaaacttcccctttcatttgtcctacttcatagctaacataatcaacaggagagattctgatattgatctcgagggcgagtttgagccttcccttctgaacagtgccatctacctcctccagctgattcagcaaatctccaccttctcgattaactaccaaggccgtcccttccgtgagtcaatccgcgagaacaagggcatgtactggggcctcattgccgcgtccggtgtcgcattctcctgcgccactgaattcattcccgagctgaatgagaagttgcgcctcgtccccttcaccaacgaatttaaggtgacattgactgtgctgatgatcttcgactacggtggctgttggttgattgagaacgtcctcaagcacctgttcagtgacttccgtcccaaggacattgccattcgtcgccctgaccagctcaagcgggaggcggaacggaagttgcaagagcaagtcgacgctgaggcccagaaggagctgcaaaggaaggtctagaggttggtggtttgaagatttgtatctgtaaacatagagaggaggttgttgaattttagaaatgttcaagtggtgtgtgacatttaatacatttatttttggcttttattgaagcattcttggaaactatatgtagaaacaaattcgtatagttgaatggctcctactctgtactgtccaatcgtcgtgaggccaggtattgccttggtagagaacagtgtagactcaaatgtggcgatcgtccgatcagcttgttacgaggttagggctcgaaatgatcggcccaccataacttcttgtagctccttgtttgagaggatgcagtctacccgttatgtagacctaattatccaggatggtcgagaatacttctcaatacacagggttagaccccagatatatgatatgtcacctcagagaggggcaaagactgggtaattccaaaaaatgtgattttgcagagggtcaaagctatatcggatactgcttcttttctctgcctcatagtgaaggaaacactatattcttcatggtaggcaaagaggtaaaagtgtacgtgccccaattcggtagaaggataggccctgtttgaaaattccacattttgaccgatatatctatagaaacatatgaagtagccgcttggcccttctccatttgaagcttcgagctgacgtggacttcaaatgcaggatgctttgttcctttgtactgccatgcaatataatgttgccttgaactcgagattataatgcgaaaacctcgtagagccgatcgcagcccgagccaacatttttctataatacataggtaaacgatctgtgaattcagaaagctcccacattgtattataagcatgaatcattcaacgcgagacttcaagcttcatgaaatccttcaggaacccaacagttgaaagaccaccaattccctagatcccactgatttcgattacgacattccggattgtagtagggcatatggcgatgccgggtttgattgcaaagaatatattcccatacattgcagtacccacctttgacaatccaggattcaggtgcgtcgacgccgccgtgattacaactagtgctggaggcgccgttgtcatagaaccatttgcaggagttaccgtcaattgattcggtgggtagacttcggtctggtcgcaggcatttgaggtccttcgtccagcccacattatgtcctttcatttggatgtagttgcccttgcagttcttcttggtgtacatttccactgtccatggatggacatatggcgcatcgctggttgtggtgcttggtttcgtgttggatgttttggttgacttgaccgatgtagttttgctagtggtggtggccttggtggttgtggtgcttgacgcagcactggccgatatcgttttattggatggggacaaggcaagtctctgaatagtcataaattttgcaaaagcattaaccgcattagtaggcatggcagtcttttttgaggtggtcgctccaatggtgcggttggtagaagtacagaattccgaagtgcttccacaataaccatacgaagagcactatgaatttattagatgttgaagagcaggatttagagcctcttgactcacacattcattagctgcacatggattcaaagaacccagatcagaccagttgctagggcgctttgtcccaggcacccgggggccacaaacagcattggaaagcgtagctggcatcatgggttctccagaactgaggcatataacagcaccctgcactaaatggtcacatccaagccattcccaggtttgagcattgtaggtctcgatatcagccaaggcgagagagtgttcttgagcaatcgtggtgcaggtttcgcccgcctggacaacatatttgtgacaaagaccatcactacctgcttgaggtgcacctgatattgctgtcggactggccaaaaggacgcctatgagagtcacgaagttgcttggtcggaagcaccacatcatattgactgatggagagtagttgctttgcttgtctttattttgcaaggcaggtttcatctttatctgttcaaagaggaaaacatgtgccaaactgccaaggatagatgcacgcatgaatatgacattgccggggaggggcaaatgtttgtgaaagaactaggatactgtgccagggccattagcatagtattgaagcaaattatagaatggcactgcatcaaaatgtggaatcctcgaattttttctttgtcttctaacgcctagtgcatgtctttccaggttgtccttgaaggctttgtctggtctcccagaaatggaaggactcaagggtatgtatacagcttctaaaacgtaaatgattcacccgagaaaggaattcataatccgaggaaggtcagacacataaggctgtctcgaaaccccttgaatgcccaaggaagaaaggaaattcctacggctgggtcagactagcaagaaaacgtcacttgacttctgagatccactcagatagcagaagaacgtgtttggtgattttcgttctttgtaaatgcataggaccagatgattcgaggaatcttcttgttagcacccttaatccaaatcttctgtagaccaagcactcggctattgatactgtttcgagagtctgtaagatatgacattactctgatacagatacgtggaatggaaacattgcgggctttcgaatgacattgggttgactaacgaaggccccttcacgcagtgacgaggccccaaagttcaaggccaacgcgcaaagcgggaccaacatcgaactccccattctcggggagctgagggcccgccttgattttgacatcttcccatttgtcaaagtcattattgaacgcctgcgtcatttccgaagcattcagaatgcgggccaaccggaagtgacaatctaggagatcagcacttggcagacttcgtacattgtctgcgtccttgttgctgaaaaccacctctatcggaaaaaagaccttgtaatttgaagaaattccttcgaacgtgtgaactttgtatttattgtccacatcctttgaaatttcggttagcaaggtaataaagtaaaatcattccacaaatgggagcgcactcactgtgggttcaaaggcaagacggaacgcgccgaagtgttcatgtacccagctagtaagggtgagaccgttggataagctgttgatctcttggtgcctgaaattcatttcccggatcctgggaaaacagcgccagaggacctcccaagctcgtgacgcatttggtatgagatcgttcttgagcccgtgagtcattgatcttaatgaggatccgagaacttactcgccggggatcccagactgcatatgacaagggaattatgtgtgcgctttctgtattcccgagtttttcaatatcctctgatttgcctagactgtcccaccgatcgagatccatgtcaccagtgacaacacagcagtaaccgtcgcgttttagtaaaccttccttgaactcccctgtccgggacataggctggtccaaagtggaatttttcttcgcatgggaatcctgccgttcggttgacgagatagttactgatgggggcttagaccgagaacgcactatttcgagtgaacgtataagtcagggctttgtacttttttgttaacttactacgggttcttagaccagtgtacaggttgcgaaagtattgataaattccatcatcgtcttccaggctgatgatatccttcgcaatggagcttttgccgcgatctgggaggaaattcaaaaaagcttgtaatgatttttgcaggatgtcttcgcgttctccacggctcttgggtttgtaactttctattcttaagcgtgcggtttcaatatcctgcttttgttcggtttccgatgcttcagtagattgtgaaaacagcgattgtgcgcggggataggcacgtgctggaccagaaacttcgctctcttgggtttgctgtttacgtttttgagaccgggtggtgggtcgatctgattcaggcaattcagaacggcgccttttaagattagttttggatggtggcgacgaggaagttggagggatgttttggttcggggcatcggcttgtagtgaagggggcggtcgctttgctggtttctttttcttccccatgatgtctgctagtagtagtatatttcttgctttccttttccaatactgagatggtagtttcagtggatgaaaatgagaacaatgggataattcagtggatggaaatgagaacaatgtgatgatgggggagaaaagtgatgtgggggtgtcgggggatagctccgagatattcctccggcagaatcgctccaccgaaaaacagtccgccggacgggtcatcccccttttggagaaaatgtatttgtagttacagaaaggcattagcccacagaacaagaattcatccatatttcattgttttccatcaagcaattactcgtccaatcgtctctcggagggtgcagagaataggctctctctggaaggccgctggaaaaagtgggaaaaggatacattctgtggccacaggcgtgggacagggtttccccctgacactgggggagaaatgtggaaatgtgggggaactctgcggagacggaagaacaaaaggcggtcaactgctgcctccacgtgatgtcacgtggagcttagccgtccagcttggaagataaccctagaggaatatgagcatattctacggagaactactccgtacaacatacggagtactcatacaactctgtagcaacccctgatgtgatctgtatttgaagtgtggacctgataccgactgctcctcaaaccccttaaacccgtatcgagtactccgtaatatgtacaccgttcactgactcacattgattaatcacattagatctctcgttttcatgtacgtggatcattatgagttcgagcattgaatataagctaaaaccataccccctgaccctaaggggccttctggaaagaaaaatcttgtcttttgcaaatcaaaatatatatagagttgtttacccgaactgtcgggttatgcatcttcaggcctgtggagctgtgtcatcattttgttactcccccttatctaccgcaggatcgccaaaatgcctagcgagactgctacaggtgactttggtccagcgccgcctgggatagacttgacagagaaccaaactggcgacttgctaggagcagtgattcctgtagcggtggtcgcgacgactgcggtgatattgcggacgattgcgccgacgaggatcaaagagatccgacaaacagctattgatgactatctcattgttgcggcgcttttattctcttggggaacggcaatatcatgcttcatcagtgagttgaccatgaggccaaagccgatgggcccagtactcacaacagactctttaggcattccatatggcaacggttatcatttgcaatctgtgacaaaagcagagtttaacactgtttggaaagtaaggaatccaatattaaatgagatgcctgggatagacgttgaccagacattcagatccttttcgcctatgtcatgatttacgctacagccgttacctgcaccaaagcctcgatcgtcttattttacggccgcatcttccactttcgctggtcactggccatctgcctgtttctggtcgttggatattgggttgccattattgtcacggttgggatggcctgtcgaccactgccacatttctggttggtctacacagatccatcagcccttggtgtctgcattgatattcccacgttctttttcgcaaatggcattgctgccatggcgattgatgtgatcatactgtgcatgccgatgccagcaatataccagtctcagatgcagttgtcgcaaaaggtagcggtcgtgggtatcctactcttgggaagtttgtatgtacctctgcccgggccctcctacgagaaggactgtagctaattattctcagtgtttgcgtggcaagtatctgccggatcatcgcacttcagaatatcaccgacgggacagatacgacgtgggctatcgccccagtctttatttggtcgtccgtggaaccatttgttgggattatttgcgcatgcctcccaacatttgggcctttctttcggcaatggcggtccatcgctcggacgcgctcatcaactgatggcagtaccgatccaagctctgagctaccatctgagacaacgacctggctccgaagatcccgaaccaaaaaacctgccaaggactcaatattcagtatcaatgatttttgctgtgtcgatgaggtccaactaatgaacgatatcaatgccactcggtcgctgggggacgaggctgcgagtgaccatcaggacgtggagggaggctgtatcacagtccaaaaagatgtggaagtgacatgggccaagtacaagtcaggaaaaaaaaatgatctggccttcaagtatcataaaggggcttgatcagctttgcaaatatttcgacttgacacggactatatttgcgttttgtgtatatttaataaaaatagacgccactggcaatttgtaattgataaaggtaagtcttattccgtaatccataccccgtactctatacaaagtactctgtgctccgtacggagtacacggaaacaaacggggatatagtcgtggcacctttcccgtgttggcggacttgcccgtaacgtaaacactccgcagatcccttccaacacagtacataatcctgcagcgaagagcgatctgatagacgctatgtgccgtcgtgacttgttatgccaattaacggtggcagaattgtggagcaatctagcagaggaaagtttcgatgtgcatgccgagccctaaaaagtcccagtgcggagaatgtagtaatcgactggacattccatgtactttgcacgctataacatatttctatgccatatacccctctggtaatcatgtagatcctcttgcttactgcgttggctcctttgtatcgtactttccgcgtcgcagcattataagaggatagagagaccgcatgagagaatacacaagagaaatcactaattcactacctgatcccccaattcactcaacatgtctcacattcacacttccagattgcaaa

SEQ ID NO: 267 - protein, 556 aa, Penicillium coprobium PF1169
MEHETDLVSFAFSGPAFDQSKPIYIDARNPSRAFNAIQFRRLVRSLIAGLKARGVERGDCVLVQLENSVLHSALFFAIVGAGGVYMGFDVASRPHEVAHLLRVAEPRLIITAPSALTRVLEVCNNQGMSSNQVLLMDEKSIESVVQFAHGQAEQTEDLDTQTVDQPIRLESLLQYGELDWLRFEDSEESKITPAAMFLTSGTSGLPKAAIRTHHTIISHHLSVYYEVPYPVVRLMALPLYHSFGDFWGNIFPIRYGQPLYIIPRFEITALLDGIRQHHITETYMVPAMIHILNRSSLNVAESLSSLRYIGISGAPIDGYSMQQFQSLLSPDAIAGNLWGMSEVGVVFQNRYGIQPQFGSVGTLLPRYELRFVNPDTGEDVAGTPDSPGELYVRGPGLLLAYKGRTDAKDEQGWFRTGDMFHVEDGNYHVIGRTKDLIKVRGQVTQYSVAPAEIEGILRKDPSIKDAAVIGVMLPDGSSEVPRAYVVRNDTSPETTADQVAGLIQSQLASYKALDGGVVFVDDIPRIGIGKHHRAKLSQLDHQRETIASILAEPVAV

SEQ ID NO: 268 - protein, 2447 aa, Penicillium coprobium PF1169
MKATEPVAIIGTGCRFPGGASSPSKLWELLQSPRDIARKVPADRFNIDAFYHPDGDHHGTTNVKESYFLDEDIKAFDAAFFNISPTEAVAMDPQQRLLLETVYESLDAAGLRMDALQRSKTGVFCGTLRNDYNQIQAMDPQAFPAYVVTGNSPSIMANRISYYFDWQGPSMAVDTGCSSSLLAVHLGVEALQNDDCSMAVAVGSNLILSPNAYIADSKTRMLSPTGRSRMWDSKADGYGRGEGVASVVLKRLQDAINDGDPIECVIRASGANSDGRTMGITMPNPKAQQSLILATYARAGLSPQNNPEDRCQYFEAHGTGTQAGDPQEAAAINSSFFGPESVPDSTDRLYVGSIKTIIGHTEATAGLAGLIKASLSLQHGMIAPNLLMQHLNPKIKPFAAKLSVPTECIPWPAVPDGCPRRASVNSFGFGGANVHVVLESYTRSELSPSNNIPSSLPFVFSAASERTLTCVMESYATFLQEHATVSLVGLALSLWDRRSTHRHRLTLMAHSIQELKDQINTEISRRVTGKPASVVSRSNTRPRRVMGIFTGQGVQWPQMGLDLIEASPSIRKWIMNLEEALDELPLDLRPQFSLLDELSQPASSSRVNEGLLSLPLRTALQIMQVNMLRAVGIELTIVVGHSSGEIVAAYAAGVLTASDAIRIAYLRGMTIDKSRDPTGRMMAVNLTWQQAQNICALEAYSGRISVAAANSPSSVTLSGDAECLRELEWLLKSLGLTPRMLRVDTAYHSPHMKPCADPYRDAMKAYPVALSASASRWYSSVYPGEVMTGYDQQELTGEYWVENMLRPVQFSQALEAAARDAGPPDLIIEIGPHPTLRGPVLQTLSKMHSAHSAIPYLALAERGKPGLDTWATALGSSWAHLGPNVVRLTDYVSLFDPNHWPVLVESLPFYPFDHTQTYWTQSRMSSNHNHRATSPNALLGSLSPETGAEKFRWRNYLRPEELPWLADRRADSGSVFPETGYISMALEAGMIMAQTQGLRLLNVKDLTIHTQLPIQNDPIGTEVLVTVGSIHSHDGAITAWFCCEAVVSGELVQCATAKMIMHPGDSDRALLPPQGQLPQALEPVDSTEFYDSLRRADYHCTGPFSTLTGLRKRRDLATGSVPVPSNDSDEPMALHPAILDLGVQTMIAAIGGLEETLLTGPFLSRNVDSTWINPVLCASDWQGKELTVASYLTCVNGDRIRGDIDIFTMNGEKAVQLEGVSLICQPSGTAPNNLQVLSQTAWGPLEPTLKKGSRKLPATMLQLHSLREELALLYLKQARNGLTDLERSGLDFDGARLLAWMNQCIANASQEPDPVGESECLDQKIEDFTAGVSPSLLNDPGLTAIAAVGQRLPRVLRDSGLQIEAWPAIDEESQYLKEDLQVLDLEDELVSVVSQACFRFPQMNILQIGQFGGHVHSGLKKMGRTYRSFTYAGLSVSGLQAIEEDLEQPGEVSHKTLDINEDPVEQGCREQFYDMVLITAAVFLQEVAVAHVRRLLKPGGFLVLLVRTNPSTTYLNLLFGPPMRCTETGKGYCSGEPITTRRDWVELLSNGGFYGLDSFDASQESESLGDFSLLLCRTPDSPAEPQSRGDLLLLGGDAEEADCLTSELFELVQDDFVKVAHAPDLDLIEDRDLSKLTVLYLVDDRDLTNATLSELCRLMTVSKRMLVVTCEKVDHPDAGLVKGLLSTFLASERSSSLLQLLHITDPVGVTTEILATALGHFVQASAAQENPHSCGLTNIEPEIQYDGSMFRVPRQYHDHATGLRHLARRQKVTDCVDLDKGVVQILPATTDKTCEGFRLLSMADPPITASYGPTLHLRVRHSSIAAVRVAGAIFLRLVIGLDVKSNKRMIALSSHIASHVIVPDSWAWSVPDTVLEAHEQSYLRATAAALLAGYLVEQVPQSGTLVVHEADGVLQSVFHQMLTRRDGKVIFSTSKSNPDKERPMLLLHEHSTARQLSQVLPSDVSAIAILHRRGQGVYDRMLSLLPDNATRIHLQDFYLTSASTGPINADDSSLIAKAFLTACLVAYTGREGLPPNSVDSLPISRISEYPILDSQDAVVDWDSTTPVLAQIPTAGSQVQLSEKKTYILVGLGSELAHAICLWLATHGAKWILLAGSRLDSDAWWLEEVSRRGTRIAVSKINLIDGISATSLHQTIPYAFPPVVGGVLIQPPPLPDCSLSQLTIDSLRNHLHPVLKGLQQLDELYKTPTLDFWVLIGSIAGVLGHADQAMTAAMSEKMALLVRHRRAQGRPASLVHLGEIHGISSPSPSQPLWCGPVAVSQRDVDEILAEAILCGRSDSNSNAELIGGLRHQSLKCGYGECPIPKLWPFYSYTATASQDQILALIETRSTKDLVTAATSLEEKAEAVVRPLMEKIRASLNLAEDAPLSADTLIPELGIDSLIAIGLSQWFTKELSVDIGVILILSGVSVGELAHAAASKLCNVSVGKP

SEQ ID NO: 269 - protein, 509 aa, Penicillium coprobium PF1169
MDNMDNMNNTPLGFNWAWAVIISFLGLLTFSFVSPHLFPSRLTVINGGRAWDIFRTKAKKRFRSDAARLIKNGFEESPDAFRIITDNGPLLVLSPQYAREVRSDDRLSLDHFIASEFHPNIPGFEPFKLILDPKNPLNTILKSNLTQALEDLSAEVTEALSATCTDDPEWHEVSVSQTALKIIAQMASKAFIGQERCRDAKWHNIIITYTHNVYGAAQALHFWPSFLRPIVAQFLPACRTLQAQIAEAREILEPLVAQRRAERATRAAQEKPHPSGGDIIDWLEQFYGDQPYDPVAAQLLLSFAAIHGTSNLLAQALIDLCGQPELVQDLREEAVSVLGKEGWTRAALYQLKLMDSALKESQRLAPNRLLSMGRIAQGDMDLSDGLRIHRGTTLMVSAHNMWDPEIYPDPRKYDGYRFHKLRQTSGQEGQHQLVSSTPDHMGFGYGKHACPGRFFAAAQIKVALCNILLKYDIEYRGGKSPGVWGQGIHLFPDPTSRIHVRRRKEEINL

SEQ ID NO: 270 - protein, 505 aa, Penicillium coprobium PF1169
MIELKDASMGAVLLTCVLVLAGLYLIRLTLSSDQLDKFPSINPRKPWEIVNVFAQRRFQQDGPRYLEAGYAKSPIFSVVTDLGPKLVVSGAFIEEFKDEKLLDHYRSMIEDFMAEVPGFESMFLGNLHNTVLRDVISVITRELEQLLAPLSDEVSAALVDTWTDSPDWHEVALLPSMLGLIAKVSSLVFVGEPLCRHPVWLETVINFTLIRHNAILALHQCPAVLRPVLHWVLPPCQKLRREIRTARTLIDSALEKSRKNPQTEKFSSVAWVDAFAKGNKYNAAMVQLRLANASIHSSADLLVKILINLCEQPELIRDLRDEIISVLGENGWRSSTLNQLKLLDSVLKESQRLHPVTTGAFSRFTRQDIKLTNGTEIPSGTPIMVTNDVAGDASIYDDPDVFDGYRYFRMREGADKARAPFTTTGQNHLGFGYGKYACPGRFFAATEIKIALCHMLLKYEWRLVKDRPHGIVTSGFAAFRDPRASIEVRRRAVAGEELEVLTGKK

SEQ ID NO: 271 - protein, 241 aa, Penicillium coprobium PF1169
MDGWSDISSAPAGYKDVVWIADRALLAQGLGWSINYLAMIYQSRKDRTYGMAILPLCCNFAWEFVYTVIYPSQNPFERAVLTTWMVLNLYLMYTTIKFAPNEWQHAPLVQRILPVIFPVAIAAFTAGHLALAATVGVAKAVNWSAFLCFELLTAGAVCQLMSRGSSRGASYTIWVSRFLGSYIGSIFMHVRETHWPQEFDWISYPFVAWHGIMCFSLDISYVGLLWYIRRQERQGQLKKAM

SEQ ID NO: 272 - protein, 464 aa, Penicillium coprobium PF1169
MKVIIVGGSIAGLALAHCLDKANIDYVILEKKKEIAPQEGASIGIMPNGGRILEQLGLYDQIEELIEPLVRAHVTYPDGFNYTSRYPALIQQRFGYPLAFLDRQKLLQILATQPVQSSRVKLDHKVESIEVSPCGVTVITSNGHTYQGDLVVGADGVHSRVRAEMWRLADASQGNVCGNGDKAFTINYACIFGISSHVDQLDPGEQITCYNDGWSILSVIGQNGRIYWFLFIKLEKEFVYDGSHKTQLHFSREDARAHCERLAQEPLWKDVTFGQVWARCEVFQMTPLEEGVLGKWHWRNIICIGDSMHKFAPHIGQGANCAIEDAAQLSNSLHTWLSGSGKEHQLKTDDLTEILAQFAQTRLQRLGPTAMAARSAMRLHAREGLKNWILGRYFLPYAGDKPADWASRGIAGGNTLDFVEPPTRAGPGWIQFSQSGKRTSFPMAVAGLCLVSIVARIMYLKLVA

SEQ ID NO: 273 - protein, 317 aa, Penicillium coprobium PF1169
MAGSQSTAQLARLLIDISRFDKYNCLFAIFPGVWSIFLAAASRHADGDPVPLDFVLGRAGLAFMYTYMLSGAGMVWNDWIDRDIDAQVARTKNRPLASGRLSTRAALIWMLVQYAASVWLMDRMVSGQDVWTYMLPLTTGIILYPFGKRPTSRKLGVYPQYILGASSALTILPAWASVYTGRISLKDLGMRCLPLCLFLFLWTIYFNTAYSYQDIKDDCKLNVNSSYVLAGSHVRGMLLLQAIAVVLVIPWILYTSASTWLWVSWLGVWTASLGEQLYLFDVKDPSSGGKVHRRNFALGIWNVLACFVELLYASGSL

SEQ ID NO: 274 - protein, 522 aa, Penicillium coprobium PF1169
MSTQEVCLPVSQRDQVKEGPVRLHGLCEDGMCDARRTGDRSAYPLSSLDHNPLGMNVTFLLFFQTTQPEKSIGVLENGIELLLKVHPFLAGDVTRRTESSQTKYTWQIEPEASESLVQFPILRIRHYQAESFKEIQSKCLLTGTEEQEIISRLAPLPIDMDISLPRRPILRFQANVMRDGIILAMTFHHSAMDGAGAARVLGLLADCCRDPTAMSSASVSPDRQLRSEIERLVPESSSGLSRMDFSKHYCGLGDWAALLAKNWSGFVRARATELVTWRLKIPGPKIEYLKEACNTLIKGQTSFQADGRPSPGFLSSNDIVSALLAMILRQAGQLAGKSTELSIAVDMRGNFKTPAFDDYLGNMVLLTYTPIQAGRNEALVDGTDPSVELRQECLEDLTQIAARIRQSLLAVDAEYIQDALSHLHSQPDWADIGFRGVPIPLSSFRNFEIFGLDFGESLGAQPRGFQLHLPVLGGMCFILPKGQDDVASTEPWDLHLTLNRDDQLLLAKDPLFCWAIGAQAKE

SEQ ID NO: 275 - protein, 434 aa, Penicillium coprobium PF1169
MDSLLTSPLWLKIAHELALYLSFIVPTAFLIITTQKSSIIRWAWTPCLLYILYQFSLRVPSLSTSQFLKGVAAGQATVAALQCLNLLLITKLDQTDLLRANLYSPSAGLLSRLAQSCALLVNFRGIGTIWEVRNIPQHAAFVQPKGKDQSMSRKRFVLREIAIIVWQYLLLDFIYESTKGTSAEDLMRLFGPGMEIKYLDATFEQWMGRLSVGIFSWLVPSRVCLNITSRLYFLILVVLGISSPESCRPGFGRVRDVCTIRGVWGKFWHQSFRWPLTSVGNYIARDVLGLAHPSLLERYTNIFFTFFTSGVLHLVCDAILGVPPSASGAMQFFCSFPLAIMIEDGVQEIWRRATGQTKDSDRAVPFWQRLVGYLWVAVWMCVTSPFYLYPAARQHAEKNWIVPFSIVEEIGLGTAQKILLGYGLFVYWAVGGEI

SEQ ID NO: 276 - protein, 1299 aa, Penicillium coprobium PF1169
MLYRAKLVDDHQIHTASLHNPIPWQLHTYVWPFLIIWPVFFAFYLSPERYDTYIQGQEWTFVFAGSIITVQSLFWLMTKWNIDINTLFTTTRSKSIDTARLIKVVPITNAGSAEICNLIREHIGPKKTLSFLFQKRRFLFYPETRSFAPLSYALDAEPKPALKTFQQSEGFTSKAEIERVQNHYGDNTFDIPVPGFIELFQEHAVAPFFVFQIFCVGLWMLDEYWYYSLFTLFMLVMFESTVVWQRQRTLSEFRGMSIKPYDVWVYRERKWQEITSDKLLPGDLMSVNRTKEDSGVACDILLVEGSVIVNEAMLSGESTPLLKDSIQLRPGDDLIEPDGLDKLSFVHGGTKVLQVTHPNLTGDAGLKNLASNVTMPPDNGALGVVVKTGFETSQGSLVRTMIYSTERVSANNVEALLFILFLLIFAIAASWYVWQEGVIRDRKRSKLLLDCVLIITSVVPPELPMELSLAVNTSLAALSKYAIFCTEPFRIPFAGRVDIACFDKTGTLTGEDLVVDGIAGLTLGEAGSKVEADGAHTELANSSAAGPDTTLVLASAHALVKLDEGEVVGDPMEKATLEWLGWTLGKNDTLSSKGNAPVVSGRSVESVQIKRRFQFSSALKRQSTIATITTNDRNASKKTKSTFVGVKGAPETINTMLVNTPPNYEETYKHFTRNGARVLALAYKYLSSETELSQSRVNNYVREEIESELIFAGFLVLQCPLKDDAIKSVQMLNESSHRVVMITGDNPLTAVHVARKVEIVDREVLILDAPEHDNSGTKIVWRTIDDKLNLEVDPTKPLDPEILKTKDICITGYALAKFKGQKALPDLLRHTWVYARVSPKQKEEILLGLKDAGYTTLMCGDGTNDVGALKQAHVGVALLNGSQEDLTKIAEHYRNTKMKELYEKQVSMMQRFNQPAPPVPVLIAHLYPPGPTNPHYEKAMERESQRKGAAITAPGSTPEAIPTITSPGAQALQQSNLNPQQQKKQQAQAAAAGLADKLTSSMMEQELDDSEPPTIKLGDASVAAPFTSKLANVIAIPNIIRQGRCTLVATIQMYKILALNCLISAYSLSVIYLDGIKFGDGQVTISGMLMSVCFLSISRAKSVEGLSKERPQPNIFNVYIIGSVLGQFAIHIATLIYLSNYVYKHEPRDSDIDLEGEFEPSLLNSAIYLLQLIQQISTFSINYQGRPFRESIRENKGMYWGLIAASGVAFSCATEFIPELNEKLRLVPFTNEFKVTLTVLMIFDYGGCWLIENVLKHLFSDFRPKDIAIRRPDQLKREAERKLQEQVDAEAQKELQRKV

SEQ ID NO: 277 - DNA, 27 bp, Artificial Sequence
  a primer sequence for PCR
gcgcggtaccattgagacaacatggat

SEQ ID NO: 278 - DNA, 26 bp, Artificial Sequence
  a primer sequence for PCR
atttaaatagttagacaatagtatca

SEQ ID NO: 279 - DNA, 35 bp, Artificial Sequence
  a primer sequence for PCR
gcgcggggtaccatgttgtcttagcattttcatta

SEQ ID NO: 280 - DNA, 40 bp, Artificial Sequence
  a primer sequence for PCR
accacgctaaagatgggggactttgcatacccagcttcca

SEQ ID NO: 281 - DNA, 40 bp, Artificial Sequence
  a primer sequence for PCR
tggaagctgggtatgcaaagtcccccatctttagcgtggt

SEQ ID NO: 282 - DNA, 40 bp, Artificial Sequence
  a primer sequence for PCR
ggtacctctgccatgaagtcctcgatcattgaccgataat

SEQ ID NO: 283 - DNA, 40 bp, Artificial Sequence
  a primer sequence for PCR
attatcggtcaatgatcgaggacttcatggcagaggtacc

SEQ ID NO: 284 - DNA, 40 bp, Artificial Sequence
  a primer sequence for PCR
gagaggtgctagcagttgttctagttcgcgagtgatgaca

SEQ ID NO: 285 - DNA, 40 bp, Artificial Sequence
  a primer sequence for PCR
tgtcatcactcgcgaactagaacaactgctagcacctctc

SEQ ID NO: 286 - DNA, 40 bp, Artificial Sequence
  a primer sequence for PCR
cagtgctacctcatgccagtctggtgagtccgtccaagta

SEQ ID NO: 287 - DNA, 40 bp, Artificial Sequence
  a primer sequence for PCR
tacttggacggactcaccagactggcatgaggtagcactg

SEQ ID NO: 288 - DNA, 40 bp, Artificial Sequence
  a primer sequence for PCR
agtaaagcgcgaaaatgctccggttgtgactggatgcaac

SEQ ID NO: 289 - DNA, 40 bp, Artificial Sequence
  a primer sequence for PCR
gttgcatccagtcacaaccggagcattttcgcgctttact

SEQ ID NO: 290 - DNA, 20 bp, Artificial Sequence
  a primer sequence for PCR
attttccctagatcacttct

SEQ ID NO: 291 - DNA, 40 bp, Artificial Sequence
  a primer sequence for PCR
ccgaattcgagctcggtaccttgtcttagcattttcatta

SEQ ID NO: 292 - DNA, 38 bp, Artificial Sequence
  a primer sequence for PCR
ctactacagatccccgggtcgtaattttccctagatca

SEQ ID NO: 293 - DNA, 26 bp, Artificial Sequence
  a primer sequence for PCR
atttaaatgtcgtacatatgctatgt

SEQ ID NO: 294 - DNA, 26 bp, Artificial Sequence
  a primer sequence for PCR
gcggtaccacaactcaactcaatagg

SEQ ID NO: 295 - DNA, 40 bp, Artificial Sequence
  a primer sequence for PCR
ccgaattcgagctcggtacctcgctattgtcagttacaca

SEQ ID NO: 296 - DNA, 38 bp, Artificial Sequence
  a primer sequence for PCR
ctactacagatccccggggaacaatcccgacacatgaa

(19) United States
(12) Patent Application Publication
(10) Pub. No.: US 2012/0330022 A1
(43) Pub. Date: Dec. 27, 2012

(54) METHOD FOR PRODUCING PYRIPYROPENE DERIVATIVES BY THE ENZYMATIC METHOD

(76) Inventors: Kentaro Yamamoto, Kawasaki-Shi (JP); Mariko Tsuchida, Meguro-ku (JP); Kazuhiko Oyama, Higashimurayama-shi (JP); Kimihiko Goto, Yokohama-shi (JP); Masaaki Mitomi, Yokosuka-shi (JP)

(21) Appl. No.: 13/575,171

(22) PCT Filed: Jan. 19, 2011
(86) PCT No.: PCT/JP2011/050853

§ 371 Date: Sep. 13, 2012

(30) Foreign Application Priority Data
Jan. 26, 2010 (JP) 2010-014336
Jan. 26, 2010 (JP) 2010-014727

(51) Int. Cl. C12P 17/18; C07D 493/04

(52) U.S. Cl. 546/283.1; 435/119

(57) ABSTRACT

There is provided a method for producing a pyripyropene derivative represented by the following formula A by an enzyme method. The production method of the present invention allows for production of a pyripyropene derivative under simpler conditions and in shorter steps.

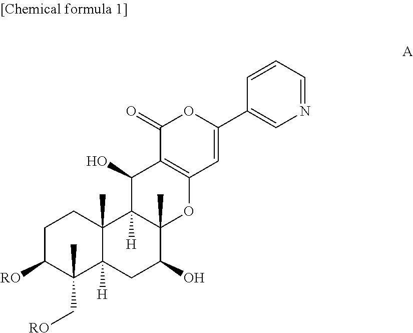

[wherein R represents a linear, branched or cyclic C₂₋₆ alkylcarbonyl group (when the alkyl moiety of this group is branched or cyclic, C₃₋₆ alkyl carbonyl group)].

### CROSS-REFERENCE TO RELATED APPLICATION

This patent application claims priority to Japanese Patent Application No. 14727/2010 that was filed on Jan. 26, 2010 and Japanese Patent Application No. 14336/2010 that was filed on Jan. 26, 2010, and the entire disclosures of all are incorporated herein by reference.

### BACKGROUND OF THE INVENTION

1. Field of Invention

The present invention relates to a method for producing a pyripyropene derivative which is useful as a pest control agent. More specifically, it relates to a method for producing a pyripyropene derivative which has acyloxy groups at the 1 position and 11 position, and which has a hydroxyl group at the 7-position.

2. Background Art

A pyripyropene derivative which has acyloxy groups at the 1 position and 11 position, and which has a hydroxyl group at the 7-position is a compound exerting a control effect against insect pests, as described in WO2006/129714 (Patent Document 1) and WO2008/066153 (Patent Document 2).

As a method for producing the pyripyropene derivative which has acyloxy groups at the 1 position and 11 position, and which has a hydroxyl group at the 7-position, a method of purification and isolation thereof from a plurality of products generated by nonselective hydrolysis of acyloxy groups using a 1,7,11-triacyloxy derivative as a raw material has been disclosed in WO2006/129714 and Japanese Patent Laid-Open Publication No. 259569/1996 (Patent Document 3).

Also, Japanese Patent Laid-Open Publication No. 259569/1996 described use of protective groups in combination for synthesis of pyripyropene derivatives. Journal of Antibiotics Vol. 49, No. 11, p. 1149, 1996 (Non-patent Document 1) and Bioorganic Medicinal Chemistry Letter Vol. 5, No. 22, p. 2683, 1995 (Non-patent Document 2) and Japanese Patent Laid-Open Publication No. 269065/1996 (Patent Document 4) have disclosed synthesis examples wherein an acyl group was introduced into the 7-position using a protective group.

WO2009/022702 (Patent Document 5) has disclosed a method for producing 1,11-diacyl-1,7,11-trideacetyl pyripyropene A from 1,7,11-trideacetyl pyripyropene A using a protective group.

Since hitherto known production of a pyripyropene derivative which has acyloxy groups at the 1 position and 11 position, and which has a hydroxyl group at the 7-position is a production method using nonselective hydrolysis of a 1,7,11-triacyloxy derivative or using protective groups in multiple steps, in industrial production, further improvement of production efficiency such as reduction of production cost, improvement of yield, facilitation of purification and isolation or shortening of the number of the steps has been desired.

### PRIOR ART REFERENCES

### Patent Documents

[Patent Document 1] WO2006/129714
[Patent Document 2] WO2008/066153
[Patent Document 3] Japanese Patent Laid-Open Publication No. 259569/1996
[Patent Document 4] Japanese Patent Laid-Open Publication No. 269065/1996
[Patent Document 5] WO2009/022702

### Non-Patent Documents

[Non-patent Document 1] Journal of Antibiotics Vol. 49, No. 11, p. 1149, 1996
[Non-patent Document 2] Bioorganic Medicinal Chemistry Letter Vol. 5, No. 22, p. 2683, 1995

### SUMMARY OF THE INVENTION

The present inventors have found a method for obtaining a desired 1,11-diacyloxy derivative under simpler conditions and in shorter steps using a pyripyropene analog, which is obtained as a naturally-occurring product (Journal of Antibiotics (1996) 49(3), 292-298, Pure Appl. Chem., vol. 71, No 6, pp. 1059-1064, 1999.; WO94/09147; Japanese Patent Laid-Open Publication No. 239385/1996; Japanese Patent Laid-Open Publication No. 259569/1996 (Patent Document 3); Bioorganic Medicinal Chemistry Letter Vol. 5, No. 22, p. 2683, 1995 (Non-patent Document 2); and WO2004/060065) as a synthetic raw material by an enzyme method by a microorganism or by a combination of an enzyme method and chemical conversion.

Thus, an object of the present invention is to provide a method for producing a 1,11-diacyloxy derivative using an enzyme method by a microorganism.

According to one embodiment of the present invention, there is provided a method for producing a compound represented by the following formula A:

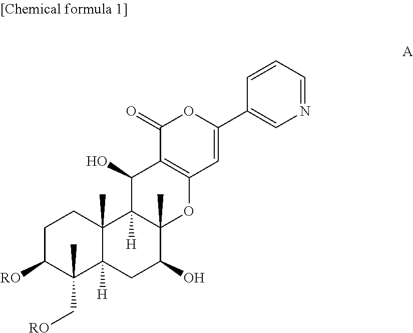

[wherein R represents a linear, branched or cyclic C₂₋₆ alkylcarbonyl group (when the alkyl moiety of this group is branched or cyclic, C₃₋₆ alkyl carbonyl group)],

the method comprising a step of culturing a microorganism into which at least one polynucleotide of (I) to (III) below or a recombinant vector comprising it/them is introduced with a compound represented by the following formula B:

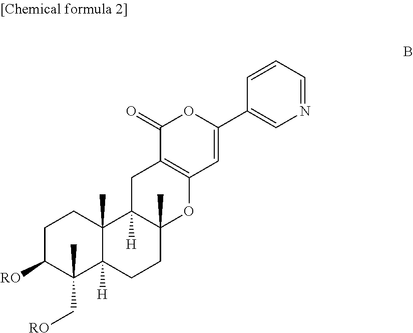

[wherein R represents the same meanings as a bove],

and isolating the compound represented by formula A:

(I) an isolated polynucleotide having at least one nucleotide sequence selected from the nucleotide sequences of the following (a) to (d):

(a) a nucleotide sequence of SEQ ID NO:266,

(b) a nucleotide sequence which is capable of hybridizing with a sequence complementary to the nucleotide sequence of SEQ ID NO:266 under stringent conditions, and which encodes a protein substantially equivalent to the protein encoded by the nucleotide sequence of SEQ ID NO:266,

(c) a nucleotide sequence of SEQ ID NO:266 in which one or more nucleotides are deleted, substituted, inserted or added, and which encodes a protein substantially equivalent to the protein encoded by the each nucleotide sequence, and

(d) a nucleotide sequence which has at least 90% identity to the polynucleotide of the nucleotide sequence of SEQ

ID NO:266, and which encodes a protein substantially equivalent to the protein encoded by the nucleotide sequence of SEQ ID NO:266;

(II) an isolated polynucleotide having a nucleotide sequence which encodes at least one amino acid sequence selected from SEQ ID NOs:269 and 270 or amino acid sequence substantially equivalent thereto; and

(III) an isolated polynucleotide having at least one nucleotide sequence selected from the nucleotide sequences of the following (1) to (4):

(1) a nucleotide sequence of the following (a) and (b):

(a) a nucleotide sequence from 13266 to 15144 of the nucleotide sequence shown in SEQ ID NO:266, and

(b) a nucleotide sequence from 16220 to 18018 of the nucleotide sequence shown in SEQ ID NO:266,

(2) a nucleotide sequence which is capable of hybridizing with a sequence complementary to the nucleotide sequence of (1) under stringent conditions, and which encodes a protein substantially equivalent to the protein encoded by the each nucleotide sequence,

(3) a nucleotide sequence of (1) in which one or more nucleotides are deleted, substituted, inserted or added, and which encodes a protein substantially equivalent to the protein encoded by the each nucleotide sequence, and

(4) a nucleotide sequence which has at least 90% identity to the polynucleotide of the nucleotide sequence of (1), and which encodes a protein substantially equivalent to the protein encoded by the each nucleotide sequence.

According to another embodiment of the present invention, there is provided the production method comprising a step of culturing a microorganism which comprises plasmid pCC1-PP1, plasmid pPP2 or plasmid pPP3, or one or more vectors selected from the group consisting of these plasmids with a compound represented by the above formula B and isolating the compound represented by the above formula A.

According to a preferred embodiment of the present invention, there is provided a method for producing a compound represented by the above formula A, the method comprising a step of culturing a microorganism into which at least one polynucleotide of (IV) to (V) below or a recombinant vector comprising it/them is introduced with a compound represented by the above formula B and isolating the compound represented by the above formula A:

(IV) an isolated polynucleotide having a nucleotide sequence which encodes the amino acid sequence of SEQ ID NO:270 or an amino acid sequence substantially equivalent thereto; and

(V) an isolated polynucleotide having at least one nucleotide sequence selected from the nucleotide sequences of the following (1) to (4):

(1) a nucleotide sequence of the following (a):

(a) a nucleotide sequence from 16220 to 18018 of the nucleotide sequence shown in SEQ ID NO:266,

(2) a nucleotide sequence which is capable of hybridizing with a sequence complementary to the nucleotide sequence of (1) under stringent conditions, and which encodes a protein substantially equivalent to the protein encoded by the each nucleotide sequence,

(3) a nucleotide sequence of (1) in which one or more nucleotides are deleted, substituted, inserted or added, which encodes a protein substantially equivalent to the protein encoded by the each nucleotide sequence, and

(4) a nucleotide sequence which has at least 90% identity to the polynucleotide of the nucleotide sequence of (1), and which encodes a protein substantially equivalent to the protein encoded by the each nucleotide sequence.

According to another embodiment of the present invention, there is provided the production method further comprising a step of acylating the hydroxyl groups at the 1 and positions of a compound represented by the following formula D using an acylating agent:

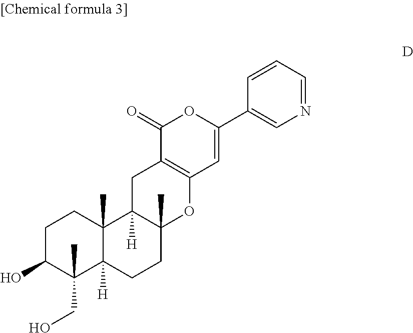

and isolating a compound represented by the above formula B.

According to another embodiment of the present invention, there is provided the production method further comprising a step of hydrolyzing the acetyl group at the 1 position and, when R′ is an acetyl group, the acetyl group at the 11 position of a compound represented by the following formula C:

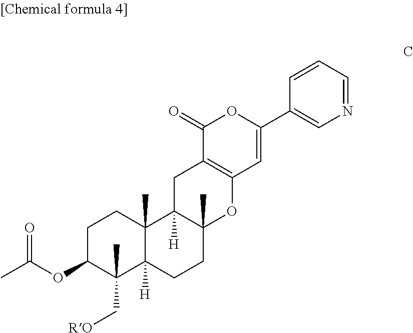

[wherein R′ represents an acetyl group or a hydrogen atom]

and isolating a compound represented by the above formula D.

According to another embodiment of the present invention, there is provided the production method further comprising a step of culturing a microorganism into which at least one polynucleotide of (VI) to (VII) below or a recombinant vector comprising it/them is introduced with pyripyropene E and isolating a compound represented by the above formula C:

(VI) an isolated polynucleotide having a nucleotide sequence which encodes the amino acid sequence of SEQ ID NOs:269 and 275 or an amino acid sequence substantially equivalent thereto; and

(VII) an isolated polynucleotide having at least one nucleotide sequence selected from the nucleotide sequences of the following (1) to (4):

(1) a nucleotide sequence of the following (a) and (b):

(a) a nucleotide sequence from 13266 to 15144 of the nucleotide sequence shown in SEQ ID NO:266, and

(b) a nucleotide sequence from 25824 to 27178 of the nucleotide sequence shown in SEQ ID NO:266,

(2) a nucleotide sequence which is capable of hybridizing with a sequence complementary to the nucleotide sequence of (1) under stringent conditions, and which encodes a protein substantially equivalent to the protein encoded by the each nucleotide sequence,

(3) a nucleotide sequence of (1) in which one or more nucleotides are deleted, substituted, inserted or added, which encodes a protein substantially equivalent to the protein encoded by the each nucleotide sequence, and

(4) a nucleotide sequence which has at least 90% identity to the polynucleotide of the nucleotide sequence of (1), and which encodes a protein substantially equivalent to the protein encoded by the each nucleotide sequence.

According to another embodiment of the present invention, there is provided the production method comprising a step of culturing a microorganism which comprises plasmid pPP2 or plasmid pPP9 with pyripyropene E and isolating a compound represented by the above formula C.

According to another embodiment of the present invention, there is provided the production method further comprising a step of culturing a microorganism into which at least one polynucleotide of (VIII) to (IX) below or a recombinant vector comprising it/them is introduced with deacetyl pyripyropene E and isolating a compound represented by the above formula C:

(VIII) an isolated polynucleotide having a nucleotide sequence which encodes at least one amino acid sequence selected from SEQ ID NOs:269, 274 and 275 or amino acid sequence substantially equivalent thereto; and

(IX) an isolated polynucleotide having at least one nucleotide sequence selected from the nucleotide sequences of the following (1) to (4):

(1) a nucleotide sequence of the following (a), (b) and (c):

(a) a nucleotide sequence from 13266 to 15144 of the nucleotide sequence shown in SEQ ID NO:266,

(b) a nucleotide sequence from 23205 to 24773 of the nucleotide sequence shown in SEQ ID NO:266, and

(c) a nucleotide sequence from 25824 to 27178 of the nucleotide sequence shown in SEQ ID NO:266,

(2) a nucleotide sequence which is capable of hybridizing with a sequence complementary to the nucleotide sequence of (1) under stringent conditions, and which encodes a protein substantially equivalent to the protein encoded by the each nucleotide sequence,

(3) a nucleotide sequence of (1) in which one or more nucleotides are deleted, substituted, inserted or added, which encodes a protein substantially equivalent to the protein encoded by the each nucleotide sequence, and

(4) a nucleotide sequence which has at least 90% identity to the polynucleotide of the nucleotide sequence of (1), and which encodes a protein substantially equivalent to the protein encoded by the each nucleotide sequence.

According to another embodiment of the present invention, there is provided the production method comprising a step of culturing a microorganism which comprises plasmid pPP2 or plasmid pPP9 and also comprises plasmid pPP7 with deacetyl pyripyropene E and isolating a compound represented by the above formula C.

According to another embodiment of the present invention, there is provided use of plasmid pCC1-PP1 (Accession No. FERM BP-11133 of Escherichia coli EPI300™-T1® transformed with plasmid pCC1-PP1), plasmid pPP2 (Accession No. FERM BP-11137 of Aspergillus oryzae transformed with plasmid pPP2), plasmid pPP3 (Accession No. FERM BP-11141 of Aspergillus oryzae transformed with plasmid pPP3), plasmid pPP7 (Accession No. FERM BP-11219 of Aspergillus oryzae transformed with plasmid pPP7), or plasmid pPP9 (Accession No. FERM BP-11220 of Aspergillus oryzae transformed with plasmid pPP9), or one or more vectors selected from the group consisting of these plasmids, for producing a compound represented by formula A.

According to a preferred embodiment of the present invention, there is provided use of plasmid pPP3 (Accession No. FERM BP-11141 of Aspergillus oryzae transformed with plasmid pPP3) or a vector comprising this plasmid, for producing a compound represented by formula A.

According to another embodiment of the present invention, there is provided use of a transformant comprising plasmid pCC1-PP1 (Accession No. FERM BP-11133 of Escherichia coli EPI300™-T1® transformed with plasmid pCC1-PP1), plasmid pPP2 (Accession No. FERM BP-11137 of Aspergillus oryzae transformed with plasmid pPP2), plasmid pPP3 (Accession No. FERM BP-11141 of Aspergillus oryzae transformed with plasmid pPP3), plasmid pPP7 (Accession No. FERM BP-11219 of Aspergillus oryzae transformed with plasmid pPP7), or plasmid pPP9 (Accession No. FERM BP-11220 of Aspergillus oryzae transformed with plasmid pPP9), or a vector selected from the group consisting of these plasmids, for producing a compound represented by formula A.

According to a preferred embodiment of the present invention, there is provided use of a transformant comprising plasmid pPP2 (Accession No. FERM BP-11137 of Aspergillus oryzae transformed with plasmid pPP2), plasmid pPP3 (Accession No. FERM BP-11141 of Aspergillus oryzae transformed with plasmid pPP3), plasmid pPP7 (Accession No. FERM BP-11219 of Aspergillus oryzae transformed with plasmid pPP7), or plasmid pPP9 (Accession No. FERM BP-11220 of Aspergillus oryzae transformed with plasmid pPP9), or a vector comprising one of these plasmids, for producing a compound represented by formula A.

According to another embodiment of the present invention, there is provided a compound represented by the following formula B1:

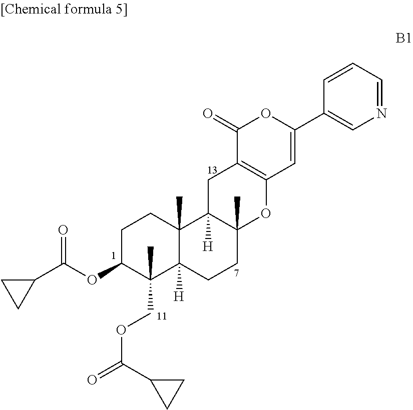

According to another embodiment of the present invention, there is provided a compound represented by the above formula D.

According to another embodiment of the present invention, there is provided a method for producing a compound represented by the above formula A from a compound represented by the above formula B, the method comprising a step of using a protein comprising the amino acid sequence described in SEQ ID NO:270, instead of a microorganism.

The present invention allows for production of a pyripyropene derivative which is useful as a pest control agent and has acyloxy groups at the 1 and 11 positions and a hydroxyl group at the 7 position under simpler conditions and in shorter steps.

### BRIEF DESCRIPTION OF DRAWINGS

FIG. 1 shows an electrophoresis pattern of PCR products by agarose gel. For the electrophoresis, the PCR products amplified using the following primers were used: M: molecular weight marker (100 bp ladder), lane 1: primers of SEQ ID NOs:1 and 2, lane 2: primers of SEQ ID NOs:239 and 240, lane 3: primers of SEQ ID NOs:237 and 238, lane 4: primers of SEQ ID NOs:241 and 242, lane 5: primers of SEQ ID NOs:247 and 248, lane 6: primers of SEQ ID NOs:251 and 252, lane 7: primers of SEQ ID NOs:245 and 246, lane 8: primers of SEQ ID NOs:243 and 244, lane 9: primers of SEQ ID NOs:249 and 250, lane 10: primers of SEQ ID NOs:235 and 236, lane 11: primers of SEQ ID NOs:233 and 234, lane 12: primers of SEQ ID NOs:227 and 228, lane 13: primers of SEQ ID NOs:229 and 230, lane 14: primers of SEQ ID NOs:231 and 232.

FIG. 2 Similarly to FIG. 1, FIG. 2 shows an electrophoresis pattern of PCR products by agarose gel. For the electrophoresis, the PCR products amplified using the following primers were used: M: molecular weight marker (100 bp ladder), lane 1: primers of SEQ ID NOs:253 and 254, lane 2: primers of SEQ ID NOs:257 and 258, lane 3: primers of SEQ ID NOs:259 and 260, lane 4: primers of SEQ ID NOs:255 and 256, lane 5: primers of SEQ ID NOs:261 and 262.

FIG. 3 Similarly to FIG. 1, FIG. 3 shows an electrophoresis pattern of PCR products by agarose gel. For the electrophoresis, the PCR products amplified using the following primers were used: lane 1: molecular weight marker (100 bp ladder), lane 2: primers of SEQ ID NOs:264 and 265 (400 bp amplified fragment).

FIG. 4 shows the plasmid map of pUSA.

FIG. 5 shows the plasmid map of pPP2.

FIG. 6 shows a scheme of P450-2 cDNA amplification.

FIG. 7 shows the plasmid map of pPP3.

FIG. 8 shows ¹H-NMR spectrum of pyripyropene E in deuterated acetonitrile.

FIG. 9 shows ¹H-NMR spectrum in deuterated acetonitrile of a product of the culture of Aspergillus oryzae transformed with plasmid pPP2.

FIG. 10 shows ¹H-NMR spectrum of pyripyropene O in deuterated acetonitrile.

FIG. 11 shows ¹H-NMR spectrum in deuterated acetonitrile of a product of the culture of Aspergillus oryzae transformed with plasmid pPP3.

FIG. 12 shows the plasmid map of plasmids pPP7 and pPP9.

### DETAILED DESCRIPTION OF THE INVENTION

### Deposition of Microorganisms

Escherichia coli (Escherichia coli EPI300™-T1®) transformed with plasmid pCC1-PP1 has been deposited with International Patent Organism Depositary, National Institute of Advanced Industrial Science and Technology (Address: AIST Tsukuba Central 6, 1-1-1 Higashi, Tsukuba, Ibaraki, Japan, 305-8566), under accession No. FERM BP-11133 (converted from domestic deposition under accession No. FERM P-21704) (identification reference by the depositors: Escherichia coli EPI300™-T1®/pCC1-PP1) as of Oct. 9, 2008 (original deposition date).

Aspergillus oryzae transformed with plasmid pPP2 has been deposited with International Patent Organism Depositary, National Institute of Advanced Industrial Science and Technology (Address: AIST Tsukuba Central 6, 1-1-1 Higashi, Tsukuba, Ibaraki, Japan, 305-8566), under accession No. FERM BP-11137 (identification reference by the depositors: Aspergillus oryzae PP2-1) as of Jun. 23, 2009.

Aspergillus oryzae transformed with plasmid pPP3 has been deposited with International Patent Organism Depositary,. National Institute of Advanced Industrial Science and Technology (Address: AIST Tsukuba Central 6, 1-1-1 Higashi, Tsukuba, Ibaraki, Japan, 305-8566), under accession No. FERM BP-11141 (identification reference by the depositors: Aspergillus oryzae PP3-2) as of Jul. 3, 2009.

Aspergillus oryzae transformed with plasmid pPP7 has been deposited with International Patent Organism Depositary, National Institute of Advanced Industrial Science and Technology (Address: AIST Tsukuba Central 6, 1-1-1 Higashi, Tsukuba, Ibaraki, Japan, 305-8566), under accession No. FERM BP-11219 (identification reference by the depositors: Aspergillus oryzae PP7) as of Dec. 21, 2009.

Aspergillus oryzae transformed with plasmid pPP9 has been deposited with International Patent Organism Depositary, National Institute of Advanced Industrial Science and Technology (Address: AIST Tsukuba Central 6, 1-1-1 Higashi, Tsukuba, Ibaraki, Japan, 305-8566), under accession No. FERM BP-11220 (identification reference by the depositors: Aspergillus oryzae PP9) as of Dec. 21, 2009.

The microorganism used in the present invention may be introduced with a polynucleotide using the recombinant vector described below. However, the polynucleotide may be introduced into the microorganism, for example, by an electroporation method, a polyethylene glycol method, an Agrobacterium method, a lithium method, a calcium chloride method or the like.

The microorganism used in the present invention is not particularly restricted as long as it can be introduced with a polynucleotide or a recombinant vector comprising it/them. Microorganisms belonging to the genus Aspergillus are preferred and more preferred microorganism includes Aspergillus oryzae.

In the present invention, culturing microorganisms can be carried out, for example, by solid culturing under aerobic conditions, shake culturing, culturing with bubbling under stirring or deep part aerobic culturing, in particular, deep part aerobic culturing is preferred. As a medium for culturing microorganisms, commonly used components, for example, as carbon sources, glucose, sucrose, starch syrup, dextrin, starch, glycerol, molasses, animal and vegetable oils or the like, can be used. Also, as nitrogen sources, soybean flour, wheat germ, corn steep liquor, cotton seed meal, meat extract, polypeptone, malto extract, yeast extract, ammonium sulfate, sodium nitrate, urea or the like can be used. Besides, as required, addition of sodium, potassium, calcium, magnesium, cobalt, chlorine, phosphoric acid (dipotassium hydrogen phosphate or the like), sulfuric acid (magnesium sulfate or the like) or inorganic salts which can generate other ions is effective. Also, as required, various vitamins such as thiamin (thiamine hydrochloride or the like), amino acids such as glutamic acid (sodium glutamate or the like) or asparagine (DL-asparagine or the like), trace nutrients such as nucleotides or selection agents such as antibiotics can be added. Further, organic substances or inorganic substances which help the growth of a bacterium and promote the production of the compound represented by formula A can be appropriately added.

The pH of the medium is, for example, about pH 5.5 to pH 8. The appropriate temperature for the culturing is 15° C. to 40° C. and, in many cases, the growth takes place around 22° C. to 30° C. The production of the compound represented by formula A varies depending on the medium and culturing conditions, or the used host. In any method for culturing, the accumulation usually reaches a peak in 2 days to 10 days. The culturing is terminated at the time when the amount of the compound represented by formula A in the culture reaches the peak and a desired substance is isolated and purified from the culture.

To isolate the compound represented by formula A from the culture, it can be extracted and purified by a usual separation means using properties thereof, such as a solvent extraction method, an ion exchange resin method, an adsorption or distribution column chromatography method, a gel filtration method, dialysis, a precipitation method, which may be individually used or appropriately used in combination. The solvent extraction method is preferred.

In the present invention, the term “substantially equivalent amino acid sequence” means an amino acid sequence which does not affect an activity of a polypeptide despite the fact that one or more amino acids are altered by substitution, deletion, addition, or insertion. Preferably, an amino acid sequence which is altered by amino acid substitution, deletion, addition, or insertion has a sequence identity of 70% or more, preferably 80% or more, more preferably 90% or more, still more preferably 95% or more, and still more preferably 98% or more to the amino acid sequence before alteration and the like. Further, the number of the altered amino acid residues is preferably 1 to 40, more preferably 1 to 20, still more preferably 1 to 10, still more preferably 1 to 8, and most preferably 1 to 4.

Further, an example of the alteration which does not affect the activity includes conservative substitution. The term “conservative substitution” means substitution of preferably 1 to 40, more preferably 1 to 20, more preferably 1 to 10, still more preferably 1 to 8, and most preferably 1 to 4 amino acid residues with other chemically similar amino acid residues such that the activity of the polypeptide is not substantially altered. Examples thereof include cases where a certain hydrophobic amino acid residue is substituted with another hydrophobic amino acid residue and cases where a certain polar amino acid residue is substituted with another polar amino acid residue having the same charges. Functionally similar amino acids capable of such a substitution are known in the art for each amino acid. Concretely, examples of non-polar (hydrophobic) amino acids include alanine, valine, isoleucine, leucine, proline, tryptophan, phenylalanine, methionine and the like. Examples of polar (neutral) amino acids include glycine, serine, threonine, tyrosine, glutamine, asparagine, cysteine and the like. Examples of positively charged (basic) amino acids include arginine, histidine, lysine and the like. Examples of negatively charged (acidic) amino acids include aspartic acid, glutamic acid and the like.

The term, “stringent conditions” in the present invention means conditions where a washing operation of membranes after hybridization is carried out at high temperatures in a solution with low salt concentrations, a person skilled in the art would be able to appropriately determine the condition, for example, the condition includes the condition of washing in a solution with 2×SSC (1×SSC: 15 mM trisodium citrate, 150 mM sodium chloride) and 0.5% SDS at 60° C. for 20 minutes, and the condition of washing in a solution with 0.2×SSC (1×SSC: 15 mM trisodium citrate, 150 mM sodium chloride) and 0.1% SDS at 60° C. for 15 minutes.

Hybridization can be carried out in accordance with a known method. Also, when a commercially-available library is used, it can be carried out in accordance with a method in the attached instructions.

In the present description, the term “identity” (also referred to as homology) for nucleotide sequences means a degree of match of bases constituting each sequence among the sequences to be compared. At that time, the presence of a gap(s) and characteristics of the amino acids are taken into account. Any values of the “identity” shown in the present description may be values calculated using a homology search program known to those skilled in the art. For instance, the value can be readily calculated by using default (initial setting) parameters in FASTA, BLAST or the like.

In the present description, the “identity” for nucleotide sequences is 90% or more, preferably 95% or more, more preferably 98% or more, still more preferably 99% or more.

In the present description, the term, “one or more nucleotides are deleted, substituted, inserted or added in a polynucleotide” means that alteration was made by a known method such as a site specific mutagenesis method, or substitution or the like of a plurality nucleotides in a degree at which they may naturally occur. The number of the altered nucleotides is one or several nucleotides (for example, one to several nucleotides or 1, 2, 3 or 4 nucleotides).

The term “nucleotide sequence which encodes a protein substantially equivalent to the protein encoded by the (each) nucleotide sequence” means a nucleotide sequence encoding a protein which has an activity equivalent to that of “the protein encoded by the (each) nucleotide sequence.”

It is preferred that a protein substantially equivalent to a protein encoded by the nucleotide sequence from 13266 to 15144 of the nucleotide sequence shown in SEQ ID NO:266 have Cytochrome P450 monooxygenase (1) (P450-1) activity.

It is preferred that a protein substantially equivalent to a protein encoded by the nucleotide sequence from 16220 to 18018 of the nucleotide sequence shown in SEQ ID NO:266 have Cytochrome P450 monooxygenase (2) (P450-2) activity.

It is preferred that a protein substantially equivalent to a protein encoded by a nucleotide sequence from 23205 to 24773 of a nucleotide sequence shown in SEQ ID NO: 266 have Acetyltransferase (AT) activity.

It is preferred that a protein substantially equivalent to a protein encoded by a nucleotide sequence from 25824 to 27178 of a nucleotide sequence shown in SEQ ID NO: 266 have Acetyltransferase-2 (AT-2) activity.

Obtainment of Isolated Polynucleotide

The method for obtaining the isolated polynucleotide of the present invention is not particularly restricted. The polynucleotide can be isolated from Penicillium coprobium PF1169 strain (Journal of Technical Disclosure 500997/2008) or filamentous bacterium by the following method. Concretely, based on a homology sequence obtained by the method of Example 9 below or the like, primers capable of specifically amplifying any one or more genes of a polyketide synthase gene, prenyltransferase gene, hydroxylase gene, acetyltransferase gene or adenylate synthetase gene, which are involved in synthesis of pyripyropene, are synthesized. PCR is carried out for a fosmid genomic library of Penicillium coprobium PF1169 strain which is separately prepared and further colony hybridization is carried out, thereby obtaining the isolated polynucleotide used in the present invention.

Recombinant Vector

The recombinant vector according to the present invention can be prepared by modifying any one or more of the polynucleotides in the above-mentioned (I) to (III) into an appropriate form depending on an object and ligating them to a vector in accordance with a conventional method, for example, gene recombination techniques described in [Sambrook, J. et al., “Molecular cloning: a laboratory manual”, (USA), 2nd Edition, Cold Spring Harbor Laboratory, 1989].

The recombinant vector used in the present invention can be appropriately selected from virus, plasmid, fosmid, cosmid vectors or the like. For instance, when a host cell is Escherichia coli, examples thereof include A phage-based bacteriophage and pBR and pUC-based plasmids. In the case of a Bacillus subtilis, examples include pUB-based plasmids. In the case of yeast, examples include YEp, YRp, YCp and YIp-based plasmids.

It is preferred that at least one plasmid among the used plasmids comprise a selection marker for selecting a transformant. As the selection marker, a gene encoding drug resistance and gene complementing auxotrophy can be used. Concrete preferred examples thereof include, when a host to be used is bacterium, ampicillin resistant genes, kanamycin resistant genes, tetracycline resistant gene and the like; in the case of yeast, tryptophan biosynthetic gene (TRP1), uracil biosynthetic gene (URA3), leucine biosynthetic gene (LEU2) and the like; in the case of a fungus, hygromycin resistant genes, bialaphos resistant genes, bleomycin resistant genes, aureobasidin resistant genes and the like; and in the case of a plant, kanamycin resistant genes, bialaphos resistant genes and the like.

In addition, DNA molecules serving as an expression vector used in the present invention preferably has DNA sequences necessary to express each gene, for example, transcription regulatory signals and translation regulatory signals such as promoters, transcription initiation signals, liposome binding sites, translation stop signals, terminators. Preferred examples of the promoters include promoters of lactose operon, tryptophan operon and the like in Escherichia coli; promoters of alcohol dehydrogenase gene, acid phosphatase gene, galactose metabolizing gene, glyceraldehyde 3-phosphate dehydrogenase gene or the like in yeast; promoters of α-amylase gene, glucoamylase gene, cellobiohydrolase gene, glyceraldehyde 3-phosphate dehydrogenase gene, abp1 gene or the like in fungi; a CaMV 35S RNA promoter, a CaMV 19S RNA promoter or a nopaline synthetase gene promoter in plants.

Transformant

A host in which the isolated polynucleotide according to the present invention is introduced may be appropriately selected, depending on the type of the used vector, from actinomycetes, Escherichia coli, Bacillus subtilis, yeast, filamentous fungus, plant cells or the like.

A method of introducing a recombinant vector into a host may be selected, depending on a host cell under test, from conjugal transfer, transduction by phage, as well as methods of transformation such as a calcium ion method, a lithium ion method, an electroporation method, a PEG method, an Agrobacterium method or a particle gun method.

When a plurality of genes is introduced into host cells in the present invention, the genes may be comprised in a single DNA molecule or individually in different DNA molecules. Further, when a host cell is a bacterium, each gene can be designed so as to be expressed as polycistronic mRNA and made into one DNA molecule.

Production Method

In the present description, the term “alkyl” as a substituent group or part thereof individually means a linear, branched, cyclic alkyl or a combination thereof unless otherwise defined.

In the present description, the symbol “C_{a-b}” affixed to a substituent group means the number of the carbon atoms comprised in the substituent group is from a to b. In the case of “C_{a-b} alkyl carbonyl”, the symbol “C_{a-b}” means the number of the carbon atoms comprised in the alkyl moiety with the carbon atoms of the carbonyl moiety being excluded is from a to b.

Concrete examples of the linear, branched, cyclic C₂₋₆ alkyl carbonyl group (C₃₋₆ alkyl carbonyl group when an alkyl moiety of this group is branched or cyclic) represented by R include a cyclopropane carbonyl group, a propionyl group and the like.

Pyripyropene E can be produced, for example, by a method for culturing microorganisms based on the method described in Japanese Patent Laid-Open Publication No. 239385/1996, WO94/09147 or U.S. Pat. No. 5,597,835; or the total synthesis method described in Tetrahedron Letters, vol. 37, No. 36, 6461-6464, 1996.

1. Production of Compound Represented by formula C

Among the compounds represented by formula C, pyripyropene O wherein R′ is an acetyl group can be obtained, for example, by a method for culturing microorganisms based on the method described in Journal of Antibiotics (1996) 49(3), 292-298 or WO94/09147.

Among the compounds represented by formula C, 11-deacetylpyripyropene O wherein R′ is a hydrogen atom can be synthesized, for example, by the method described in Reference Example 1 below.

According to a preferred embodiment of the present invention, it is preferred to use a microorganism to produce a compound represented by the above formula C. When using a microorganism, one can use the culture medium itself, or one can use a microbial cell suspension obtained by isolating microbial cells from the culture medium and washing them, followed by suspending the obtained living microbial cells in water, physiological saline, or appropriate buffer at a prescribed concentration. The compound can be obtained by adding deacetyl pyripyropene E or pyripyropene E to a suspension of the microorganism used or the microbial cell suspension thereof and allowing the resultant to react in the presence of an enzyme at the optimum temperature and pH for an appropriate period of time. The microorganism can be cultured as described above. A preferred concentration of deacetyl pyripyropene E or pyripyropene E to be added ranges from 1 μg/mL to 50,000 μg/mL.

When adding pyripyropene E, a protein having a function of hydroxylation (hereinafter referred to as hydroxylase) can be used instead of a microorganism. When using the hydroxylase, the compound can be obtained by dissolving pyripyropene E in a solution of the hydroxylase in water or appropriate buffer and allowing the resultant to react in the presence of an enzyme at the optimum temperature and pH for an appropriate period of time.

When adding deacetyl pyripyropene E, a protein having a function of acetylation (hereinafter referred to as acetylase) and hydroxylase can be used instead of a microorganism. When using acetylase and hydroxylase, the compound can be obtained by dissolving deacetyl pyripyropene E in a solution of acetylase and hydroxylase in water or appropriate buffer and allowing the resultant to react in the presence of an enzyme at the optimum temperature and pH for an appropriate period of time.

Both when adding pyripyropene E and when adding deacetyl pyripyropene E, preferred reaction conditions are a temperature from 10° C. to 40° C., a pH from 5 to 9, and a reaction time from 30 minutes to 24 hours. More preferred conditions are a temperature from 20° C. to 35° C., a pH from 6 to 8, and a reaction time from an hour to 12 hours.

As the acetylase, a purified acetylase may be used, or a crude enzyme solution obtained by disrupting bacteria, actinomycetes, yeast or fungi containing acetylase can be used. Further, a crude enzyme solution in the form of a solution of disrupted fungi and the like can also be used. As the acetylase, preferably, an acetylase derived from a microorganism having the ability to produce pyripyropene, such as, for example, Aspergillus fumigatus strain FO-1289 (Japanese Patent Laid-Open Publication No. 360895/1992), Eupenicillium reticulosporum strain NRRL-3446 (Applied and Environmental Microbiology (1995), 61(12), 4429-35), Penicillium griseofulvum strain F1959 (WO2004/060065) and Penicillium coprobium strain PF1169 (Journal of Technical Disclosure 500997/2008) can be used.

As the hydroxylase, a purified hydroxylase may be used, or a crude enzyme solution obtained by disrupting bacteria, actinomycetes, yeast or fungi containing hydroxylase can be used. Further, a crude enzyme solution in the form of a solution of disrupted fungi and the like can also be used. As the hydroxylase, preferably, a hydroxylase derived from a microorganism having the ability to produce pyripyropene, such as, for example, Aspergillus fumigatus strain FO-1289 (Japanese Patent Laid-Open Publication No. 360895/1992), Eupenicillium reticulosporum strain NRRL-3446 (Applied and Environmental Microbiology (1995), 61(12), 4429-35), Penicillium griseofulvum strain F1959 (WO2004/060065), and Penicillium coprobium strain PF1169 (Journal of Technical Disclosure 500997/2008) can be used.

A compound represented by the above formula C which is generated by conversion can be isolated as described above.

According to a preferred embodiment of the present invention, one can culture a microorganism into which at least one polynucleotide described of the above (VI) to (VII) or a recombinant vector comprising it/them is introduced with pyripyropene E and isolate a compound represented by the above formula C.

According to another preferred embodiment of the present invention, one can culture a microorganism which comprises plasmid pPP2 or plasmid pPP9 with pyripyropene E and isolate a compound represented by the above formula C.

An isolated compound represented by the above formula C which is obtained by culturing a microorganism which comprises plasmid pPP2 with pyripyropene E is preferably, but not limited to, 11-deacetyl pyripyropene O wherein R′ is a hydrogen atom in the above formula C. Further, an isolated compound represented by the above formula C which is obtained by culturing a microorganism which comprises plasmid pPP9 with pyripyropene E is preferably, but not limited to, a compound wherein R′ is an acetyl group in the above formula C.

According to another preferred embodiment of the present invention, one can culture a microorganism into which at least one polynucleotide of the above (VIII) to (IX) or a recombinant vector comprising it/them is introduced with deacetyl pyripyropene E and isolate a compound represented by the above formula C.

According to another embodiment of the present invention, one can culture a microorganism which comprises plasmid pPP2 or plasmid pPP9 and also comprises plasmid pPP7 with deacetyl pyripyropene E, and isolate a compound represented by the above formula C.

An isolated compound represented by the above formula C which is obtained by culturing a microorganism which comprises plasmid pPP2 with deacetyl pyripyropene E is preferably, but not limited to, 11-deacetyl pyripyropene O wherein R′ is a hydrogen atom in the above formula C. Further, an isolated compound represented by the above formula C which is obtained by culturing a microorganism which comprises plasmid pPP9 with deacetyl pyripyropene E is preferably, but not limited to, a compound wherein R′ is an acetyl group in the above formula C.

According to another preferred embodiment of the present invention, one can culture a microorganism into which at least one polynucleotide of the above (VIII) to (IX) or a recombinant vector comprising it/them is introduced with deacetyl pyripyropene E, and isolate a compound represented by the above formula C.

2. Method for Producing a Compound Represented by Formula B (Hereinafter Sometimes Referred to as Compound B) from a Compound Represented by Formula C (Hereinafter Referred to as Compound C, Including Pyripyropene O wherein R′ is Acetyl Group and 11-Deacetyl pyripyropene O wherein R′ is Hydrogen Atom)

Compound B is obtained by hydrolyzing compound C and then acylating the resulting compound represented by the above formula D (hereinafter sometimes referred to as Compound D).

The hydrolysis of Compound C can be carried out under conditions where an acid or base is used. Concrete examples of the acid which can be used include hydrochloric acid, sulfuric acid, p-toluenesulfonic acid, p-toluenesulfonic acid monohydrate, pyridinium p-toluenesulfonate, 10-camphorsulfonic acid and the like. Concrete examples of the base which can be used include inorganic bases such as sodium carbonate, potassium carbonate, sodium hydrogen carbonate, potassium bicarbonate, sodium hydroxide, potassium hydroxide, sodium hydride, potassium hydride, sodium cyanide, potassium cyanide, magnesium hydroxide, calcium hydroxide, lithium hydroxide or barium hydroxide; alkoxides of alkali metals or alkaline earth metals such as sodium methoxide, sodium ethoxide or tert-butoxypotassium; and organic bases such as triethylamine, diisopropylethylamine, pyridine, hydrazine or guanidine. The hydrolysis can be carried out in an appropriate solvent. Concrete examples of the solvent which can be used include alcohol solvents having 1 to 4 carbon atoms such as methanol; ether solvents such as diethyl ether, diisopropyl ether, tetrahydrofuran or dioxane; aprotic polar organic solvents such as N,N-dimethylformamide, dimethyl sulfoxide, N,N-dimethylacetamide or acetonitrile; halogenated solvents such as dichloromethane or chloroform; or water; and mixtures thereof.

Examples of a solvent which can be used in a method for obtaining Compound B by acylating Compound D (1,1′-dideacetyl pyripyropene O) include ether solvents such as diethyl ether, diisopropyl ether, tetrahydrofuran or dioxane; aprotic polar organic solvents such as N,N-dimethylformamide, dimethyl sulfoxide, N,N-dimethylacetamide or acetonitrile; halogenated solvents such as dichloromethane or chloroform; aromatic hydrocarbon solvents such as toluene; and mixtures thereof.

The reaction can be carried out without using a base. However, in the case of using the base, examples of the base which can be used include inorganic bases such as sodium carbonate, potassium carbonate, sodium hydrogen carbonate, potassium bicarbonate, sodium hydroxide, potassium hydroxide, sodium hydride, potassium hydride, sodium cyanide, potassium cyanide, magnesium hydroxide, calcium hydroxide, lithium hydroxide or barium hydroxide; and organic bases such as triethylamine, diisopropylethylamine, pyridine or guanidine.

As an acylation agent corresponding to a desired R, ROH, RCl, (R)₂O or mixed acid anhydride can be used with RCl, (R)₂O being preferred. The reaction can be carried out in the presence or absence of a base, or by using a condensing agent such as dicyclohexylcarbodiimide, 1-ethyl-3-(3-dimethylaminopropyl)carbodiimide hydrochloride, carbonyldiimidazole, dipyridyl disulfide, diimidazolyl disulfide, 1,3,5-trichlorobenzoylchloride, 1,3,5-trichlorobenzoyl anhydride, PyBop or PyBrop; and preferably carried out in the presence or absence of a base by using RCl or (R)₂O. An example of a preferred acylation agent includes cyclopropanecarbonyl chloride.

When the base is used, the amount thereof is preferably 2 to 10 equivalents, more preferably 3 to 6 equivalents, based on Compound D.

The amount of the acylation agent to be used is preferably 2 to 10 equivalents, more preferably 2 to 5 equivalents, based on Compound D. It is preferred that the reaction temperature be within a range of −20° C. to 50° C. It is preferred that the reaction time be within a range 0.1 hour to 7 days.

3. Production of Compound Represented by Formula A (Hereinafter, Described as Compound A) from Compound B

When the production is carried out using a microorganism, a culture medium per se can be used or a fungal cell suspension obtained by suspending living fungal cells in water, physiological saline or an appropriate buffer at a prescribed concentration, which living fungal cells is obtained by isolating fungal cells from the culture medium and washing them, can also be used. Compound B can be added to the culture medium of the used microorganism or the fungal cell suspension thereof and allowed to react at the optimum temperature and pH of an enzyme for an appropriate period of time, thereby obtaining Compound A. A method for culturing microorganisms can be carried out in accordance with the above. A preferred concentration of the compound B to be added is 1 μg/mL to 50,000 μg/mL.

When the production is carried out by, instead of the microorganism, a protein having a function of hydroxylation (hereinafter, referred to as hydroxylase), Compound B can be dissolved in an enzyme solution obtained by dissolving the hydroxylase in water or an appropriate buffer and allowed to react at the optimum temperature and pH of the enzyme for an appropriate period of time, thereby obtaining Compound A.

As a preferred reaction condition, the temperature is 10° C. to 40° C.; the pH is pH 5 to pH 9; and the reaction time is 30 minutes to 24 hours. As a more preferred reaction condition, the temperature is 20° C. to 35° C.; the pH is pH 6 to pH 8; and the reaction time is 1 hour to 12 hours. As the hydroxylase, a purified hydroxylase may be used or a crude enzyme solution obtained by disrupting bacteria, actinomycetes, yeast or fungi containing the hydroxylase can be used. Further, a crude enzyme solution in the form of a solution of disrupted fungi and the like can also be used. As the hydroxylase, preferably hydroxylases derived from microorganisms having ability to produce pyripyropene, Aspergillus fumigatus FO-1289 strain (Japanese Patent Laid-Open Publication No. 360895/1992), Eupenicillium reticulosporum NRRL-3446 strain (Applied and Environmental Microbiology (1995), 61(12), 4429-35), Penicillium oriseofulvum F1959 strain (WO2004/060065) and Penicillium coprobium PF1169 strain (Journal of Technical Disclosure 500997/2008) can be used. Compound A generated by conversion can be isolated in accordance with the above.

According to the present invention, there is provided a method for producing a compound represented by the above formula A, the method comprising a step of culturing a microorganism into which at least one polynucleotide of the above (I) to (III) or a recombinant vector comprising it/them is introduced with a compound represented by the above formula B and isolating the compound represented by the above formula A.

According to a preferred embodiment of the present invention, there is provided the production method wherein the compound represented by the above formula B is a compound represented by the above formula B1.

There is provided the production method comprising a step of culturing a microorganism which comprises plasmid pCC1-PP1, plasmid pPP2 or plasmid pPP3, or one or more vectors selected from the group consisting of these plasmids with a compound represented by the above formula B and isolating the compound represented by the above formula A.

According to a more preferred embodiment of the present invention, there is provided a method for producing a compound represented by the above formula A, the method comprising a step of culturing a microorganism into which at least one polynucleotide of the above (IV) to (V) or a recombinant vector comprising it/them is introduced with a compound represented by the above formula B and isolating the compound represented by the above formula A.

According to a preferred embodiment of the present invention, there is provided a method for producing 1,11-di-O-cyclopropanecarbonyl-1,7,11-tri-deacetyl pyripyropene A by acylating the hydroxyl groups at the 1 and 11 positions of a compound represented by the above formula D using an acylating agent to produce a compound represented by the above formula B1 and culturing a microorganism which comprises plasmid pPP3 with the compound represented by the above formula B1 (1,1′-di-O-cyclopropanecarbonyl-1,11-dideacetyl pyripyropene O)

### EXAMPLES

The present invention will be further illustrated in detail by the following examples, which are not intended to restrict the present invention.

### Example 1

### Preparation of Genomic DNA of Penicillium coprobium PF1169 Strain

A sterilized NB medium (500 ml) was placed in an Erlenmeyer flask (1 L). Penicillium coprobium PF1169 strain (Journal of Technical Disclosure No. 500997/2008) precultured in ½ CMMY agar medium at 28° C. for 4 days was added to the above-mentioned medium and subjected to liquid culture at 28° C. for 4 days. Filtration was carried out with Miracloth to obtain 5 g of fungal cells. From these fungal cells, 30 μg of genomic DNA was obtained in accordance with the manual attached to genomic DNA purification kit Genomic-tip 100/G (manufactured by Qiagen K.K.).

### Example 2

### Degenerate primers for Amplification of Polyketide Synthase (PKS) and Amplified Fragment Thereof

Based on an amino acid sequence conserved among various filamentous bacterium polyketide synthases, the following primers were designed and synthesized as degenerate primers for amplification:

(SEQ ID NO: 1)

| | |
|---|---|
| LC1: | GAYCCIMGITTYTTYAAYATG |

(SEQ ID NO: 2)

| | |
|---|---|
| LC2c: | GTICCIGTICCRTGCATYTC |

(wherein R = A/G, Y = C/T, M = A/C, I = inosine).

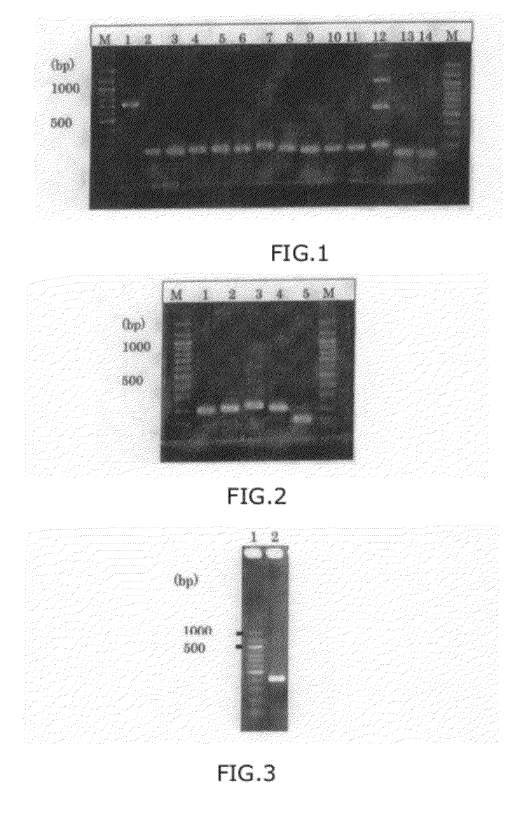

Using these degenerate primers, the genomic DNA prepared in Example 1 and ExTaq polymerase (manufactured by Takara Bio Inc.) were allowed to react in accordance with the attached manual. An amplified fragment of about 700 bp was detected (see FIG. 1). Further then, the above-mentioned amplified fragment was analyzed to specify the sequence of its internal 500 bp (SEQ ID NO:3).

### Example 3

### Large-Scale Sequencing of Genomic DNA and Amino Acid Sequence Homology Search

The genomic DNA of Penicillium coprobium PF1169 strain obtained in Example 1 was subjected to large-scale sequencing and homology search for amino acid sequences. Specifically, part of 50 μg of genomic DNA was pretreated and thereafter subjected to Roche 454FLX DNA sequencer to obtain about 250 bp, 103 thousands of fragment sequences (in total, 49 Mb of sequence).

For these sequences, as known sequences among polyketide synthases and prenyltransferases, the following five sequences (sequences derived from polyketide synthases: Aspergillus(A.) fumigatus PKS 2146a.a. and Penicillium(P.) oriseofluvum 6-methylsalycilic acid synthase 1744 a.a.; as well as prenyltransferases: Aspergillus (A.) fumigatus Prenyltransferase, Aspergillus(A.) fumigatus Prenyltransferase (4-hydroxybezoate octaprenyltransferase) and Penicillium(P.) marneffei Prenyltransferase) were selected and search by homology sequence search software blastx was carried out, thereby obtaining 89, 86, 2, 1 and 3 of homology sequences, respectively (see Table 1). Further, from the homology sequences of A. fumigatus PKS 2146a.a. and P. qriseofluvum 6-methylsalycilic acid synthase 1744 a.a., 19 and 23 of contig sequences were respectively obtained (the contig sequences of A. fumigatus PKS 2146a.a.: SEQ ID NOs:179 to 197; the contig sequences of P. oriseofluvum 6-methylsalycilic acid synthase 1744 a.a.: SEQ ID NOs:198 to 220) (see Table 1).

TABLE 1

| | | Number of | |
|---|---|---|---|
| | | Homology | SEQ |
| Enzyme Name | Origin | Sequences | ID NO. |
| Polyketide | A. fumigatus | 89 | 4 to 92 |
| Syntheses | PKS 2146 a.a. | | |
| | P. griseofluvum | 86 | 93 to 178 |
| | 6-methylsalycilic acid | | |
| | synthase 1744 a.a. | | |
| | A. fumigatus | 19 (Contig | 179 to 197 |
| | PKS 2146 a.a. | sequences) | |
| | P. griseofluvum | 23 (Contig | 198 to 220 |
| | 6-methylsalycilic acid | sequences) | |
| | synthase 1744 a.a. | | |
| Prenyl- | A. fumigatus | 2 | 221, 222 |
| transferases | Prenyltransferase | | |
| | A. fumigatus | 1 | 223 |
| | Prenyltransferase | | |
| | (4-hydroxybezoate | | |
| | octaprenyltransferase) | | |
| | P. marneffei | 3 | 224 to 226 |
| | Prenyltransferase | | |

### Example 4

### PCR Amplification from Genomic DNA

From the search results of blastx obtained in Example 3, for polyketide synthases, 13 types of primer pairs shown in SEQ ID NOs:227 to 252 were synthesized. Similarly, for prenyltransferases, 5 types of primer pairs shown in SEQ ID NOs:253 to 262 were synthesized. When PCR was carried out for the genomic DNA using these primers, amplified fragments with the expected size were seen for all of the primer pairs (see FIG. 1 and FIG. 2).

### Example 5

### Construction of Phage Genomic Library

A A phage genomic library of Penicillium coprobium PF1169 strain was constructed using λBIueSTAR XhoI Half-site Arms Kit (manufactured by Takara Bio Inc., Cat. No. 69242-3) in accordance with the attached manual. That is, genomic DNA was partially digested using a restriction enzyme, Sau3A1. The DNA fragment with about 20 kb (0.5 μg) was ligated to 0.5 μg of λBlueSTAR DNA attached to the kit. This ligation solution was subjected to in vitro packaging using Lambda INN Packaging kit (manufactured by Nippon Gene Co., Ltd.) based on the manual attached to the kit to obtain 1 ml of a solution. This solution with packaged phages (10 μl) was infected into 100 μl of E. coli ER1647 strain and cultured on a plaque-forming medium at 37° C. overnight, thereby obtaining about 500 clones of plaques. Thus, the genomic library composed of about 50000 clones of phages in which 10 to 20 kb genomic DNA of Penicillium coprobium PF1169 strain were introduced by infection was constructed.

### Example 6

### Screening from Phage Library

For 10000 clones of the phage library prepared in Example 5, the primary screening was carried out by plaque hybridization using, as a probe, the PCR product amplified by LC1-LC2c primer pair prepared above. For labeling and detection of the probe, AlkPhos Direct Labelling and Detection System with CDP-Star (manufactured by GE Healthcare, Cat. No. RPN3690) was used. The above-mentioned hybridization was carried out in accordance with the attached manual.

By the primary screening, 6 clones remained as candidates. Further, as the result of the secondary screening by plaque hybridization, 4 clones were obtained. These positive clones were infected into E. coli BM25.8 strain and the phages were converted to plasmids in accordance with the attached manual, thereby obtaining 4 types of plasmids containing a desired region.

### Example 7

### Preparation of Fosmid Genome Library

A genomic library of Penicillium coprobium PF1169 strain was constructed in accordance with the manual attached to CopyControl Fosmid Library Production Kit (manufactured by EPICENTRE, Cat. No. CCFOS110). That is, 0.25 μg of DNA fragment of about 40 kb genomic DNA was blunt-ended and then incorporated into fosmid vector pCCFOS (manufactured by Epicentre). This ligation solution was subjected to in vitro packaging using MaxPlax Lambda Packaging Extract attached to the kit based on the manual attached to the kit. This solution with packaged virus (10 μl) was infected into 100 μl of E. coli EPI300™-T1® strain and cultured on a medium containing chloramphenicol at 37° C. overnight and selected, thereby obtaining 300 clones of colonies. Thus, about 30000 clones of the fosmids in which 40 kb the genomic DNA of Penicillium coprobium PF1169 strain were introduced by infection were obtained. They were aliquoted in a 96 well plate so as to be about 50 clones per well. Thus, the genomic library composed of 96 pools, about 4800 clones constructed.

### Example 8

### Fosmid Library Screening

In accordance with the manual attached to the fosmid, plasmid DNAs were individually prepared from 96 pools of the library prepared in Example 7. Using the degenerate primers for polyketide synthase amplification synthesized in Example 2, PCR was carried out for 96 pools of these plasmid DNA samples. As a result, DNA fragments of about 700 bp were amplified from 9 pools. Further, a petri dish containing colonies of about 300 clones or more was prepared from the positive pools and re-screening was carried out by colony hybridization. As a result, by using LC1-LC2c primer pair, 9 types of fosmids were obtained from about 4800 clones.

### Example 9

### Large-Scale Sequencing of Genomic DNA and Amino Acid Sequence Homology Search

Genomic DNA of Penicillium coprobium PF1169 strain obtained in Example 1 was subjected to large-scale sequencing and homology search for amino acid sequences. Specifically, part of 50 μg of genomic DNA was pretreated and then subjected to Roche 454FLX DNA sequencer to obtain 1405 fragment sequences with an average contig length of 19.621 kb (sequence of a total base length of 27.568160 Mb).

For these sequences, as known sequences among polyketide synthases and prenyltransferases, the following five sequences (sequences derived from polyketide synthases: Penicillium(P.) griseofluvum 6-methylsalycilic acid synthase 1744 a.a. (P22367) and Aspergillus(A.) fumigatus PKS 2146 a.a. (Q4WZA8); as well as prenyltransferases: Penicillium (P.) marneffei Prenyltransferase (Q0MR08), Aspergillus (A.) fumigatus Prenyltransferase (Q4WBI5) and Aspergillus(A.) fumigatus Prenyltransferase (4-hydroxybezoate octaprenyltransferase) (Q4WLD0)) were selected and search by homology sequence search software blastx was carried out, thereby obtaining 22 (P22367), 21 (Q4WZA8), 2 (Q0MR08), 3 (Q4WBI5) and 3 (Q4WLD0) of the homologous sequences, respectively.

### Example 10

### Fosmid Library Screening and Sequence Analysis of Cluster Genes

In accordance with the manual attached to a fosmid kit (manufactured by EPICENTRE, CopyControl Fosmid Library Production Kit), plasmid DNAs were individually prepared from 96 pools of the library prepared in Example 7. Based on base sequences determined by Roche 454FLX DNA sequencer, homology search for amino acid sequences was carried out to search regions adjacent to polyketide synthase and prenyltransferase. Based on the base sequence of prenyltransferase of the obtained region, a primer pair (No. 27) capable of amplifying 400 bp DNA fragment was synthesized. Using the primers, PCR was carried out for these 48 pools of plasmid DNA samples. As a result, expected DNA fragments of about 400 bp (SEQ ID NO:263) were amplified from 11 pools (see FIG. 3). Further, a petri dish containing colonies of about 300 clones or more was prepared from 6 pools of the positive pools and re-screening was carried out by colony hybridization. As a result, by using 27F+27R primer pair (27F primer: SEQ ID NO:264, 27R primer: SEQ ID NO:265), 4 types of fosmids were obtained from about 4800 clones. One of them was named pCC1-PP1 and the entire sequence of the inserted fragment was determined (SEQ ID NO:266).

The obtained pCC1-PP1 was transformed into Escherichia coli EPI300™-T1® strain (attached to the fosmid kit), thereby obtaining Escherichia coli EPI300™-m-T1® strain/pCC1-PP1.

When a homology search was carried out between the above-mentioned sequence of SEQ ID NO:266 and each of CoA ligase; LovB-like polyketide synthase (PKS); Cytochrome P450 monooxygenase, Cyclase (IMP: Integral membrane protein), FAD-dependent monooxygenase (FMO), which are hydroxylases; UbiA-like prenyltransferase (UbiAPT); Acetyltransferase (AT), Acetyltransferase-2 (AT-2), which are acetyltransferases; and Cation transporting ATPase (the above-mentioned enzymes are all derived from Aspergillus fumigatus Af293 strain), a high homology of 70% or more was seen in any search.

The nucleotides 3342 to 5158 of SEQ ID NO:266 encode CoA ligase and the corresponding polypeptide is shown with the amino acid sequence depicted in SEQ ID NO:267; the nucleotides 5382 to 12777 of SEQ ID NO:266 encode LovB-like polyketide synthase (PKS) and the corresponding polypeptide is shown with the amino acid sequence depicted in SEQ ID NO:268; the nucleotides 13266 to 15144 of SEQ ID NO:266 (hereinafter, a protein encoded by this polynucleotide sequence (P450-1) is referred to as Cytochrome P450 monooxygenase (1) (P450-1)) and the nucleotides 16220 to 18018 (hereinafter, a protein encoded by this polynucleotide sequence (P450-2) is referred to as Cytochrome P450 monooxygenase (2) (P450-2)) encode Cytochrome P450 monooxygenases and the corresponding polypeptides are shown with the amino acid sequences depicted in SEQ ID NOs:269 and 270, respectively; the nucleotides 18506 to 19296 of SEQ ID NO:266 encode Cyclase and the corresponding polypeptide is shown with the amino acid sequence depicted in SEQ ID NO:271; the nucleotides 19779 to 21389 of SEQ ID NO:266 encode FAD-dependent monooxygenase (FMO) and the corresponding polypeptide is shown with the amino acid sequence depicted in SEQ ID NO:272; the nucleotides 21793 to 22877 of SEQ ID NO:266 encode UbiA-like prenyltransferase (UbiAPT) and the corresponding polypeptide is shown with the amino acid sequence depicted in SEQ ID NO:273; the nucleotides 23205 to 24773 of SEQ ID NO:266 encode Acetyltransferase (AT) and the corresponding polypeptide is shown with the amino acid sequence depicted in SEQ ID NO:274; the nucleotides 25824 to 27178 of SEQ ID NO:266 encode Acetyltransferase-2 (AT-2) and the corresponding polypeptide is shown with the amino acid sequence depicted in SEQ ID NO:275; and the nucleotides 27798 to 31855 of SEQ ID NO:266 encode Cation transporting ATPase and the corresponding polypeptide is shown with the amino acid sequence depicted in SEQ ID NO:276.

### Example 11

### Hydroxylation of Pyripyropene E or Pyripyropene O by Transformation of Aspergillus Oryzae

Pyripyropene E used below was able to be produced by a method for culturing a microorganism based on the method described in Japanese Patent Laid-Open Publication No. 239385/1996, WO94/09147 or U.S. Pat. No. 5,597,835, or the total synthesis method described in Tetrahedron Letters, vol. 37, No. 36, 6461-6464, 1996. Also, pyripyropene O used below was able to be produced by a method for culturing a microorganism based on the method described in J. Antibiotics 49, 292-298, 1996 or WO94/09147.

(1) Preparation of Expression Vector for Introducing into Filamentous Bacterium

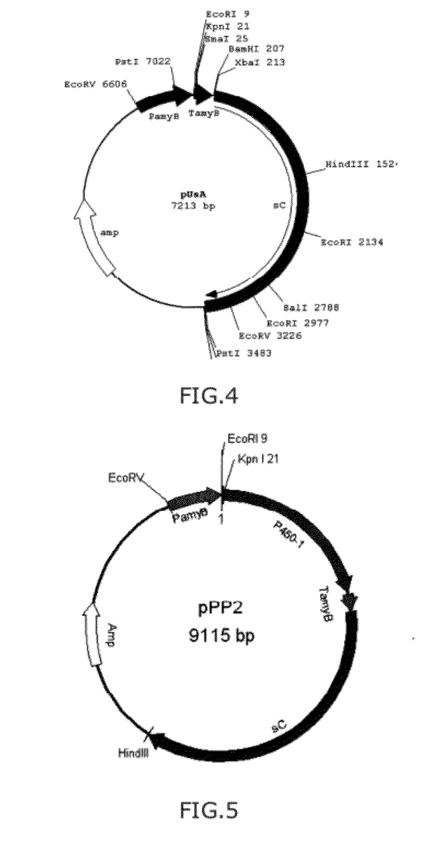

pUSA (FIG. 4) and pHSG399 (Takara Bio Inc.) were individually digested with KpnI and ligated, thereby obtaining pUSA-HSG. This plasmid was digested with SmaI and KpnI in the order mentioned, and subjected to gel purification, thereby obtaining a linear vector DNA having a KpnI cohesive end and SmaI blunt end.

(2) Preparation of Plasmid pPP2

With fosmid pCC1-PP1 as a template, the polynucleotide of the above-mentioned P450-1 was amplified using a primer pair P450-1 with Kpn F (SEQ ID NO:277)/P450-1 with Swa R (SEQ ID NO:278). The purified DNA fragment was cloned into pCR-Blunt (Invitorogen, Cat. No. K2700-20). The plasmid obtained was digested with KpnI and SwaI. The above-mentioned P450-1 fragment was ligated to the above-described vector pUSA-HSG, thereby obtaining a plasmid pPP2 shown in FIG. 5.

(3) Preparation of Plasmid pPP3

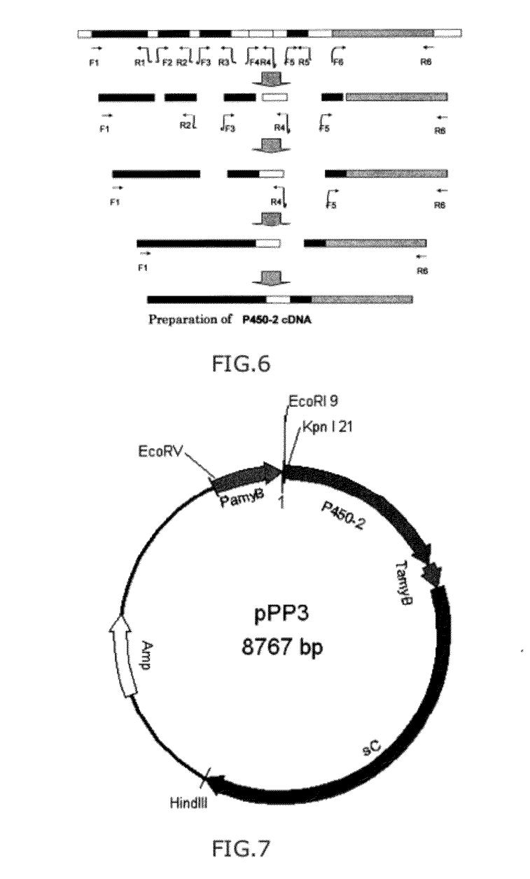

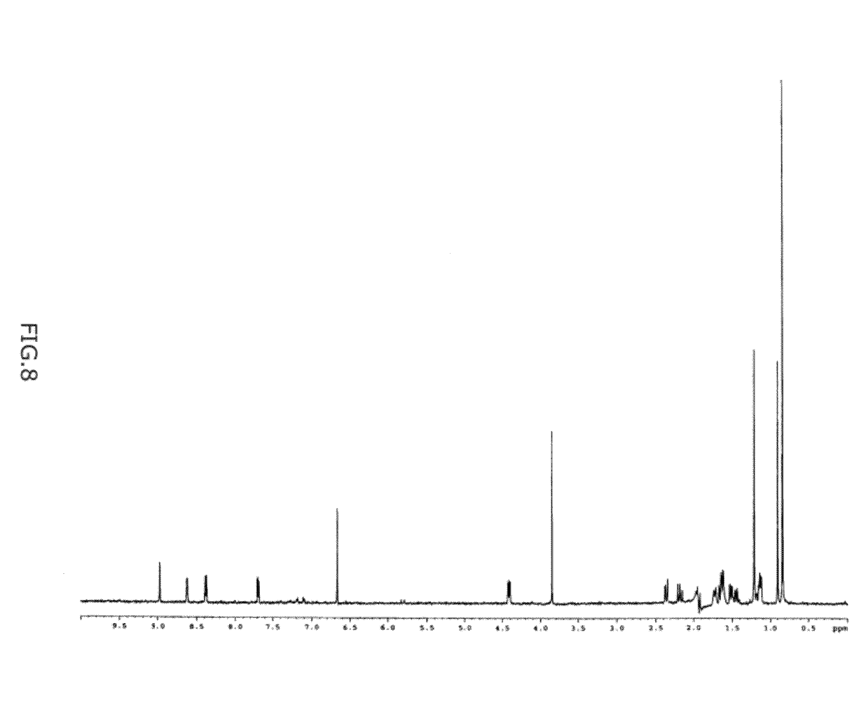

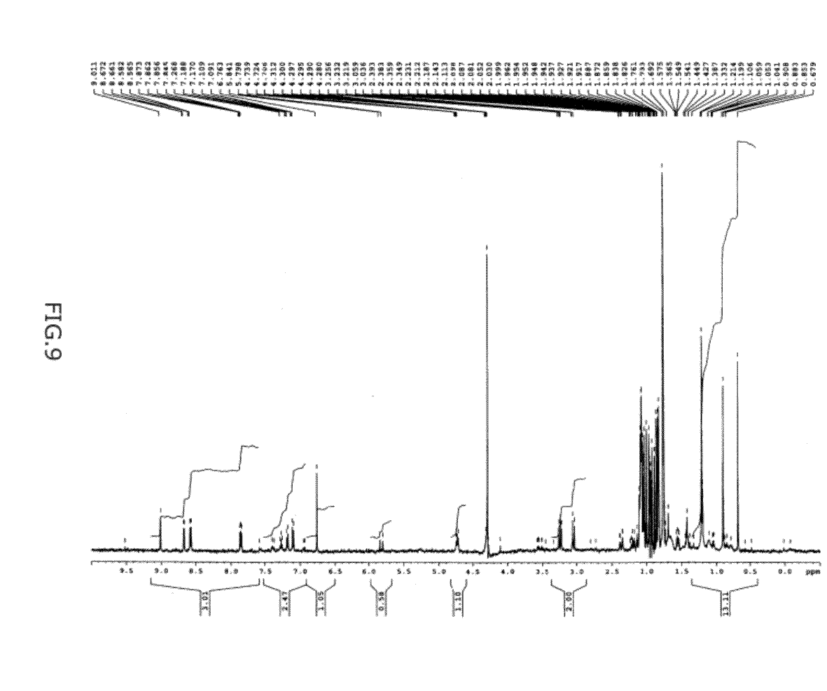

With fosmid pCC1-PP1 as a template, in accordance with the flow shown in FIG. 6, exons alone were first amplified using primer pairs F1(SEQ ID NO:279)/R1(SEQ ID NO:280), F2(SEQ ID NO:281)/R2(SEQ ID NO:282), F3(SEQ ID NO:283)/R3(SEQ ID NO:284), F4(SEQ ID NO:285)/R4(SEQ ID NO:286), F5(SEQ ID NO:287)/R5(SEQ ID NO:288) and F6(SEQ ID NO:289)/R6(SEQ ID NO:290), thereby obtaining six fragments. Next, amplification was carried out with these fragments as templates using primer pairs of F1/R2, F3/R4 and F5/R6, thereby obtaining longer fragments. Further, by repeating amplification using primer pairs of F1/R4 and F1/R6, cDNA which did not contain introns of the polynucleotide of the above-mentioned P450-2 was prepared. This cDNA fragment was inserted into pCR-Blunt (Invitorogen, Cat. No. K2700-20) and the obtained plasmid was used as a template for amplification by a primer pair, infusion F of P450-2-cDNA (SEQ ID NO:291)/infusion R of P450-2-cDNA (SEQ ID NO:292). Based on the manual of the kit, a plasmid pPP3 shown in FIG. 7 was obtained using In-Fusion Advantage PCR Cloning Kit (Clontech).

(4) Preparation of Plasmid pPP7 (AT)

Using vector pUSA-HSG for filamentous fungus transformation obtained in the above-mentioned Example 11(1), plasmid pPP7 was obtained.

With fosmid pCC1-PP1 as a template, the polynucleotide of AT was each amplified using a primer pair AT F with Swa (SEQ ID NO:293) and AT R with Kpn (SEQ ID NO:294). A purified fragment was cloned into a vector for PCR fragments, pCR-Blunt (Invitorogen, Cat. No. K2700-20). The plasmid obtained was digested with KpnI and SwaI. Each fragment was ligated between the KpnI and SmaI sites of the above-described filamentous bacterium vector pUSA-HSG, thereby obtaining a plasmid pPP7 shown in FIG. 12.

(5) Preparation of Plasmid pPP9 (AT-2)

With fosmid pCC1-PP1 as a template, Toxin fragment was amplified using a primer pair infusion F of Toxin (SEQ ID NO:295) and infusion R of Toxin (SEQ ID NO:296), and inserted between the KpnI and SmaI sites of the above-described filamentous bacterium vector pUSA-HSG using In-Fusion Advantage PCR Cloning Kit (manufactured by Clontech, Cat. No. 639619), based on the manual of the kit, thereby obtaining a plasmid pPP9 shown in FIG. 12.

(6) Transformation of Aspergillus Oryzae (A. oryzae)

In a CD-Met (containing L-Methionine 40 μg/ml) agar medium, A. oryzae (HL-1105 strain) was cultured at 30° C. for one week. From this petri dish, conidia (>10⁸) were collected and seeded in 100 ml of YPD liquid medium in a 500 ml-flask. After 20-hour culturing (30° C., 180 rpm), fungal cells having a moss ball shape were obtained. The fungal cells were collected with a 3G-1 glass filter, washed with 0.8 M NaCl, and water was removed well. The resultant was suspended with TF solution I (protoplast formation solution) and then shook at 30° C., at 60 rpm for 2 hours. At a 30-minute interval, observation under the microscope was carried out and the presence of protoplasts was checked. Thereafter, the culture medium was filtered and subjected to centrifugation (2000 rpm, 5 minutes) to collect protoplasts, which were then washed with TF solution II. After washing, 0.8 volume of TF solution II and 0.2 volume of TF solution III were added and mixed, thereby obtaining a protoplast suspension.

To 200 μl of this suspension, 10 μg of plasmid DNA (pPP2 or pPP3) was added. The mixture was left to stand on ice 30 minutes and added with TF solution III (1 mL). The resulting mixture was gently mixed and then left to stand at room temperature for 15 minutes. Thereafter, the plasmid DNA was introduced into the above-mentioned protoplasts. To this, TF solution II (8 mL) was added and subjected to centrifugation (at 2000 rpm for 5 minutes). Further, protoplasts were then recovered with 1 to 2 ml being left over. The recovered protoplast solution was dropped to a regeneration medium (lower layer) and a regeneration medium (upper layer) was poured. The resultant was mixed by turning a petri dish and then cultured at 30° C. for 4 to 5 days. Generated clones were isolated in the regeneration medium (lower layer), subcultured and purified, thereby obtaining a transformant (Aspergillus oryzae PP2-1 and Aspergillus oryzae PP3-2).

Based on the method described in the above-mentioned Example 11 (6), transformants in which each of the plasmid DNAs (pPP7 and pPP9) was introduced were obtained (Aspergillus oryzae PP7 and Aspergillus oryzae PP9).

The above-mentioned TF solution I (protoplast formation solution) was prepared with the following compositions.

| Name of Compound | Concentration | |
|---|---|---|
| Yatalase (manufactured by Takara Bio Inc.) | 20 | mg/ml |
| Ammonium sulfate | 0.6M | |
| Maleic acid-NaOH | 50 | mM |

After the above-mentioned compositions (pH 5.5) were prepared, filter sterilization was carried out.

The above-mentioned TF solution II was prepared with the following compositions.

| Name of Compound | Amount |
|---|---|
| 1.2M Sorbitol (MW = 182.17) | 43.72 g |
| 50 mM CaCl₂ | 10 ml 1M CaCl₂ ( 1/20) |
| 35 mM NaCl | 1.4 ml 5M NaCl |
| 10 mM Tris-HCl | 2 ml 1M Tris-HCl ( 1/100) |

Water was further added to attain a total volume of 200 ml.

After the above-mentioned compositions were prepared, autoclave sterilization was carried out.

The above-mentioned TF solution III was prepared with the following compositions.

| Name of Compound | Amount |
|---|---|
| 60% PEG4000 | 6 g |
| 50 mM CaCl₂ | 500 μl 1M CaCl₂ ( 1/20) |
| 50 mM Tris-HCl | 500 μl 1M Tris-HCl ( 1/100) |

Water was further added to attain a total volume of 10 ml.

After the above-mentioned compositions were prepared, filter sterilization was carried out.

The above-mentioned regeneration medium was prepared with the following compositions.

| | | |
|---|---|---|
| Name of Compound | Amount | Concentration |

| | | | |
|---|---|---|---|
| Sorbitol (MW = 182.17) | 218.6 | g | 1.2M |
| NaNO₃ | 3.0 | g | 0.3% (w/v) |
| KCl | 2.0 | g | 0.2% (w/v) |
| KH₂PO₄ | 1.0 | g | 0.1% (w/v) |

| | | |
|---|---|---|
| MgSO₄•7H₂O | 2 ml of 1M MgSO₄ | 0.05% 2 mM |

| | | | |
|---|---|---|---|
| Trace elements solution | 1 | ml | |
| Glucose | 20.0 | g | 2% (w/v) |

Water was further added to attain a total volume of 1 L. After the above-mentioned compositions (pH 5.5) were prepared, autoclave sterilization was carried out.

In addition, the Trace elements solution used above was prepared with the following composition.

| Name of Compound | Amount | |
|---|---|---|
| FeSO₄•7H₂O | 1.0 | g |
| ZnSO₄•7H₂O | 8.8 | g |
| CuSO₄•5H₂O | 0.4 | g |
| Na₂B₄O₇•10H₂O | 0.1 | g |
| (NH₄)₆Mo₇O₂₄•4H₂O | 0.05 | g |

Water was further added to attain a total volume of 1 L. After the above-mentioned compositions were prepared, autoclave sterilization was carried out.

(7) Function Analysis and Addition Culture Test of P450-1

To a YPD medium (1% (w/v) Yeast Extract, 2% (w/v) Peptone, 2% (w/v) Dextrose) containing 1% (w/v) maltose, a 1/100 volume of 2 mg/mL dimethyl sulfoxide solution of pyripyropene E was added to provide medium A. From flora of Aspergillus oryzae PP2-1 cultured in Czapek Dox agar medium, conidia thereof were collected and suspended in sterilized water. This conidia suspension was adjusted to 10⁴ spores/mL. Further, 100 μL of this adjusted conidia suspension was added to 10 mL of medium A and cultured with shaking at 25° C. for 96 hours. To this culture medium, 10 mL of acetone was added and the mixture was mixed well. Thereafter, acetone was removed using a centrifugal concentrator. To this, 10 mL of ethyl acetate was added and the resulting mixture was mixed well and then only the ethyl acetate layer was recovered. A dried product obtained by removing ethyl acetate using the centrifugal concentrator was dissolved in 1000 μL of methanol. This was used as a sample and analyzed by LC-MS (Waters, Micromass ZQ, 2996PDA, 2695 Separation module, Column: Waters XTerra C18 (φ4.5×50 mm, 5 μm)) and LC-NMR (manufactured by Burker Daltonik, Avance500).

As the results of the above-mentioned LC-MS measurement, it was confirmed that the obtained compound was single Compound E which increased by a molecular weight of 16 compared with pyripyropene E. In addition, as the results of the LC-NMR measurement, it was confirmed that this Compound E was an 11-position hydroxide of pyripyropene E. It was confirmed that the above-mentioned Cytochrome P450 monooxygenase (1) was an enzyme hydroxylating the 11-position of pyripyropene E with pyripyropene E as a substrate.

Physicochemical properties of the above-mentioned Compound E are shown below:

1. Mass spectrum: ES-MS 468M/Z (M+H)⁺

2. Molecular formula: C₂₇H₃₃NO₆

3. HPLC: Column: Waters XTerra Column C18 (5 μm, 4.6 mm×50 mm), 40° C., Mobile phase: From 20% aqueous acetonitrile solution to 100% acetonitrile in 10 minutes (linear gradient), Flow rate: 0.8 ml/min, Detection: Retention time 6.696 minutes at UV 323 nm

4. ¹H-NMR spectrum (CD₃CN, 2H, 3.134, 3.157 H-11)

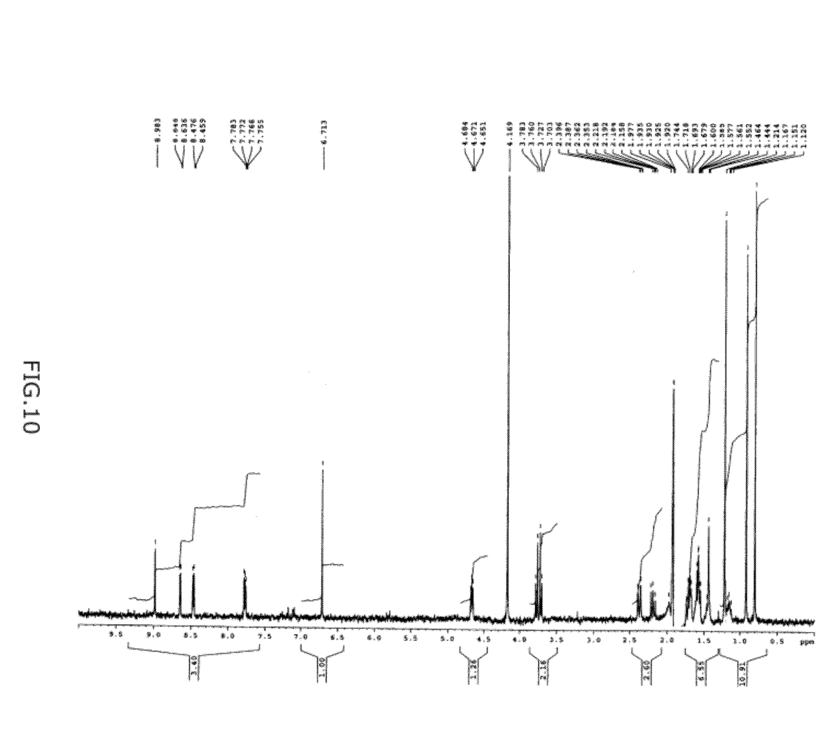

The charts of the ¹H-NMR spectrum of pyripyropene E and ¹H-NMR spectrum according to the above-mentioned 4 are shown in FIG. 8 and FIG. 9, respectively.

(8) Function Analysis and Addition Culture Test of P450-2

To a YPD medium (1% (w/v) Yeast Extract, 2% (w/v) Peptone, 2% (w/v) Dextrose) containing 1% (w/v) maltose, a 1/100 volume of 2 mg/mL dimethyl sulfoxide solution of pyripyropene E was added to provide medium A, and similarly a 1/100 volume of 2 mg/mL dimethyl sulfoxide solution of pyripyropene O was added to provide medium B. From flora of Aspergillus oryzae PP3-2 cultured in Czapek Dox agar medium, conidia thereof were collected and suspended in sterilized water. This conidia suspension was adjusted to 10⁴ spores/mL. Further, 500 μL of the adjusted conidia suspension was added to 50 mL of medium A or medium B and cultured with shaking at 25° C. for 96 hours. To this culture medium, 50 mL of acetone was added and the mixture was mixed well. Thereafter, acetone was removed using a centrifugal concentrator. To this, 50 mL of ethyl acetate was added and the resulting mixture was mixed well and then only the ethyl acetate layer was recovered. A dried product obtained by removing ethyl acetate using the centrifugal concentrator was dissolved in 1500 μL of methanol. This was used as a sample and analyzed by LC-MS (manufactured by Waters, Micromass ZQ, 2996PDA, 2695 Separation module, Column: Waters XTerra C18 (φ4.5×50 mm, μm)) and LC-NMR (manufactured by Burker Daltonik, Avance500). As the results of the LC-MS measurement, from a sample obtained from the medium A, Compound F which increased by a molecular weight of 32 compared with pyripyropene E was detected. Also, from a sample obtained from the medium B, Compound G which increased by a molecular weight of 32 compared with pyripyropene O was detected. Further, as the results of the LC-NMR measurement, it was confirmed that Compound G was a 7-position and 13-position hydroxide of pyripyropene O. It was confirmed that the above-mentioned Cytochrome P450 monooxygenase (2) had a hydroxylase activity of the 7-position and 13-position of each of pyripyropene E or pyripyropene O.

Physicochemical properties of the above-mentioned Compound F are shown below:

1. Mass spectrum: ES-MS 484M/Z (M+H)⁺

2. Molecular formula: C₂₇H₃₃NO₇

3. HPLC: Column: Waters XTerra Column C18 (5 μm, 4.6 mm×50 mm), 40° C., Mobile phase: From 20% aqueous acetonitrile solution to 100% acetonitrile in 10 minutes (linear gradient), Flow rate: 0.8 ml/min, Detection: Retention time 5.614 minutes at UV 323 nm

Physicochemical properties of the above-mentioned Compound G are shown below:

1. Mass spectrum: ES-MS 542M/Z (M+H)⁺

2. Molecular formula: C₂₉H₃₅NO₉

3. HPLC: Column: Waters XTerra Column C18 (5 μm, 4.6 mm×50 mm), 40° C., Mobile phase: From 20% aqueous acetonitrile solution to 100% acetonitrile in 10 minutes (linear gradient), Flow rate: 0.8 ml/min, Detection: Retention time 5.165 minutes at UV 323 nm

4. ¹H-NMR spectrum (CD₃CN, 1H 4.858 H-13), (CD₃CN, 1H 3.65 H-7)

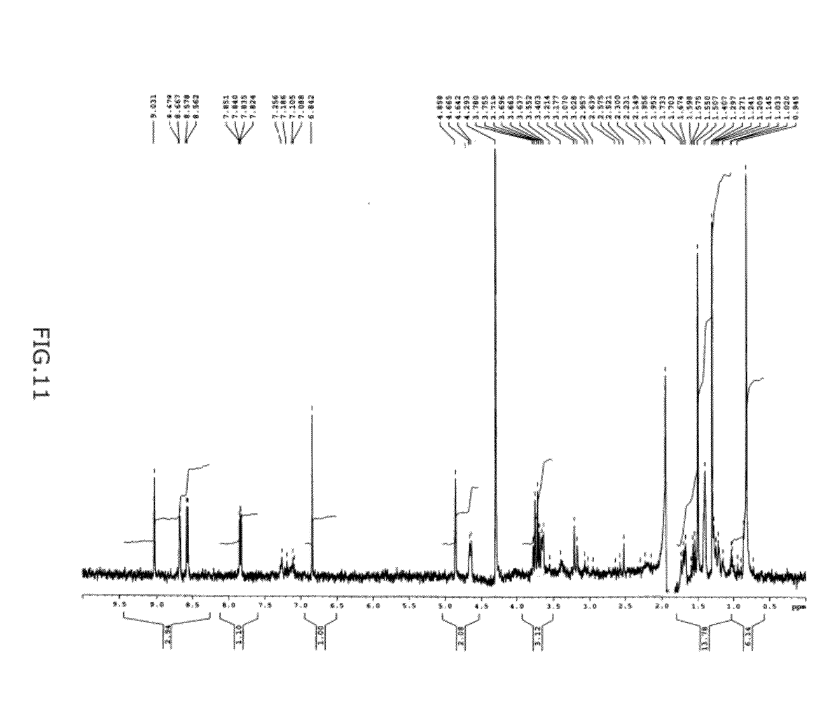

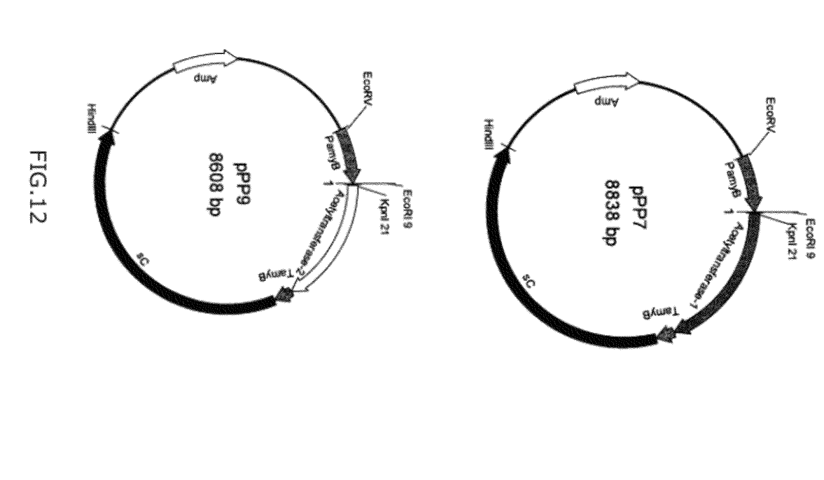

The charts of the ¹H-NMR spectrum of pyripyropene O and the above-mentioned Compound G are shown in FIG. 10 and FIG. 11, respectively.

(9) Function Analysis and Addition Culture Test of Acetyltransferase-1

To a YPD medium (1% (w/v) Yeast Extract, 2% (w/v) Peptone, 2% (w/v) Dextrose) containing 1% (w/v) maltose, a 1/100 volume of 2 mg/mL dimethyl sulfoxide solution of deacetyl pyripyropene E (see Reference Example 3) was added to provide medium D; a 1/100 volume of 2 mg/mL dimethyl sulfoxide solution of 11-deacetyl pyripyropene O (see Reference Example 4) was added to provide medium E and 2 mg/mL dimethyl sulfoxide solution of 7-deacetyl pyripyropene A (see Reference Example 5) was added to provide medium F. From flora of Aspergillus oryzae PP7 cultured in Czapek Dox agar medium, conidia thereof were collected and suspended in sterilized water. This conidia suspension was adjusted to 10⁴ spores/mL. Further, 200 μL of this was added to 20 mL of medium D, medium E or medium F and cultured with shaking at 25° C. for 96 hours. To this culture medium, 20 mL of acetone was added and the mixture was mixed well. Thereafter, acetone was removed using a centrifugal concentrator. To this, 20 mL of ethyl acetate was added and the resulting mixture was mixed well and then only the ethyl acetate layer was recovered. A dried product obtained by removing ethyl acetate using the centrifugal concentrator was dissolved in 1000 μL of methanol. This was used as a sample and analyzed by LC-MS (Waters, Micromass. ZQ, 2996PDA, 2695 Separation module, Column: Waters XTerra C18 (φ4.5×50 mm, 5 μm)).

As the results of the LC-MS measurement, a single compound which increased a molecular weight of 42 compared with deacetyl pyripyropene E was detected from the medium D. It was confirmed that the compound had the same retention time, molecular ion peaks and UV absorption as pyripyropene E (see Reference Example 6). Meanwhile, no newly-generated compounds were detected from the medium E and medium F. From this, it was confirmed that Acetyltransferase-1 had an acetyltransferase activity which acetylated specifically the 1-position of deacetyl pyripyropene E.

(10) Function Analysis and Addition Culture Test of Acetyltransferase-2

To a YPD medium (1% (w/v) Yeast Extract, 2% (w/v) Peptone, 2% (w/v) Dextrose) containing 1% (w/v) maltose, a 1/100 volume of 2 mg/mL dimethyl sulfoxide solution of deacetyl pyripyropene E (see Reference Example 3) was added to provide medium D; a 1/100 volume of 2 mg/mL dimethyl sulfoxide solution of 11-deacetyl pyripyropene O (see Reference Example 4) was added to provide medium E and 2 mg/mL dimethyl sulfoxide solution of 7-deacetyl pyripyropene A (see Reference Example 5) was added to provide medium F.

From flora of Aspergillus oryzae PP9 cultured in Czapek Dox agar medium, conidia thereof were collected and suspended in sterilized water. This conidia suspension was adjusted to 10⁴ spores/mL. Further, 200 μL of this was added to 20 mL of medium D, medium E or medium F and cultured with shaking at 25° C. for 96 hours. To this culture medium, 20 mL of acetone was added and the mixture was mixed well. Thereafter, acetone was removed using a centrifugal concentrator. To this, 20 mL of ethyl acetate was added and the resulting mixture was mixed well and then only the ethyl acetate layer was recovered. A dried product obtained by removing ethyl acetate using the centrifugal concentrator was dissolved in 1000 μL of methanol. This was used as a sample and analyzed by LC-MS (Waters, Micromass ZQ, 2996PDA, 2695 Separation module, Column: Waters XTerra C18 (φ4.5×50 mm, 5 μm)).

As the results of the LC-MS measurement, a single compound which increased a molecular weight of 42 compared with 11-deacetyl pyripyropene O was detected from the medium E. It was confirmed that this compound had the same retention time, molecular ion peaks and UV absorption as pyripyropene O (see Reference Example 7). Further, a single compound which increased a molecular weight of 42 compared with 7-deacetyl pyripyropene A was detected from the medium F. It was confirmed that the compound had the same retention time, molecular ion peaks and UV absorption as pyripyropene A (see Reference Example 8). Meanwhile, no newly-generated compounds were detected from the medium D. From this, it was confirmed that Acetyltransferase-2 had an acetyltransferase activity which acetylated specifically the 11-position of 11-deacetyl pyripyropene O and the 7-position of 7-deacetyl pyripyropene A.

### Example 12

### Synthesis and Structural Analysis of Compound D (1,11-dideacetyl pyripyropene O)

Pyripyropene O (30 mg) was dissolved in methanol-water (19:1, 2 mL) and potassium carbonate (20 mg) was added thereto. The resultant was stirred at room temperature for 22 and half hours, and thereafter acetic acid (0.1 mL) was added to concentrate. Ethyl acetate and water were added and then extraction was carried out with ethyl acetate. The ethyl acetate layer was washed with saturated sodium chloride solution and dried with anhydrous sodium sulfate. The solvent was evaporated under reduced pressure, thereby obtaining a crude product of 1,11-dideacetyl pyripyropene O. The crude product was purified by preparative thin layer chromatography (Merck silica gel 60F254, 0.5 mm, hexane:acetone=1:1), thereby obtaining 1,11-dideacetyl pyripyropene O (23 mg).

ESI-MS; 426 m/z (M+H)⁺; ¹H-NMR (CDCl₃) δ 0.89 (3H, s), 0.97 (3H, s), 1.14 (1H, dt, J=4.2, 12.8 Hz), 1.20-1.25 (1H, m), 1.28 (3H, s), 1.45-1.59 (3H, m), 1.64-1.75 (3H, m), 1.82 (1H, dt, J=3.5, 9.6 Hz), 2.11-2.14 (1H, m), 2.25 (1H, dd, J=12.8, 17.1 Hz), 2.54 (1H, dd, J=4.6, 17.1 Hz), 3.45 (1H, d, J=10.3 Hz), 3.68 (1H, dd, J=5.0, 11.2 Hz), 3.75 (1H, d, J=10.3 Hz), 6.42 (1H, s), 7.39 (1H, dd, J=4.8, 8.0 Hz), 8.10 (1H, ddd, J=1.6, 2.0, 8.0 Hz), 8.65 (1H, dd, J=1.6, 4.8 Hz), 8.99 (1H, d, J=2.0 Hz)

### Example 13

### Synthesis and Structural Analysis of Compound B1 (1,11-di-O-cyclopropanecarbonyl-1,11-dideacetyl pyripyropene O)

1,11-dideacetyl pyripyropene O (22 mg) was suspended in ethyl acetate (1 mL), and pyridine (20 mg) and cyclopropanecarbonyl chloride (22 mg) were added thereto. The resultant was stirred at room temperature for 4 hours, and water was added and then extraction was carried out with ethyl acetate. The ethyl acetate layer was washed with saturated sodium chloride solution and dried with anhydrous sodium sulfate. The solvent was evaporated under reduced pressure, thereby obtaining a crude product of 1,11-di-O-cyclopropanecarbonyl-1,11-dideacetyl pyripyropene O. The crude product was purified by preparative thin layer chromatography (Merck silica gel 60F254, 0.5 mm, chloroform:methanol=10:1), thereby obtaining 1,11-di-O-cyclopropanecarbonyl-1,11-dideacetyl pyripyropene O (17 mg).

ESI-MS; 562 m/z (M+H)⁺; ¹H-NMR (CDCl₃) δ 0.88 (3H, s), 0.99 (3H, s), 0.84-1.08 (8H, m), 1.21 (1H, dt, J=3.6, 13.4 Hz), 1.28 (3H, s), 1.43-1.48 (2H, m), 1.56-1.73 (6H, m), 1.81-1.85 (2H, m), 2.13-2.16 (1H, m), 2.26 (1H, dd, J=12.8, 17.1 Hz), 2.55 (1H, dd, J=4.6, 17.1 Hz), 3.71 (1H, d, J=11.7 Hz), 3.93 (1H, d, J=11.7 Hz), 4.82 (1H, dd, J=4.7, 12.0 Hz), 6.44 (1H, s), 7.41 (1H, dd, J=4.8, 8.0 Hz), 8.12 (1H, ddd, J=1.4, 2.0, 8.0 Hz), 8.66 (1H, dd, J=1.4, 4.8 Hz), 9.00 (1H, d, J=2.0 Hz)

### Example 14

### Synthesis of 1,11-di-O-cyclopropanecarbonyl-1,7,11-tri-deacetyl pyripyropene A

From flora of Aspergillus oryzae PP3-2 (FERM BP-11141) cultured in Czapek Dox agar medium, conidia thereof were collected and suspended in sterilized water to obtain a conidia suspension of 10⁴ spores/mL. To 20 mL of YPD medium (1% (w/v) Yeast Extract, 2% (w/v) Peptone, 2% (w/v) Dextrose) containing 1% (w/v) maltose, 200 μL of the conidia suspension and 200 μL of 2 mg/mL dimethyl sulfoxide solution of 1,11-di-O-cyclopropanecarbonyl-1,11-dideacetyl pyripyropene O obtained in Example 13 were added and the resulting mixture was cultured with shaking at 25° C. for 96 hours. To this culture medium, 20 mL of acetone was added and the mixture was mixed well. Thereafter, acetone was removed using a centrifugal concentrator. To this, 20 mL of ethyl acetate was added and the resulting mixture was mixed well and then the ethyl acetate layer was recovered. Ethyl acetate was removed using the centrifugal concentrator, thereby obtaining a product. By analyzing under the following conditions, it was confirmed that the product was the captioned compound described in WO2006/129714 (Conversion rate 90%).

LC-MS (Waters, Micromass ZQ, 2996PDA, 2695 Separation module, Column: Waters XTerra C18 (φ4.5×50 mm, 5 μm)), Mobile phase: From 20% aqueous acetonitrile solution to 100% acetonitrile in 10 minutes (linear gradient), Flow rate: 0.8 ml/min, Column oven: 40° C., Detection: UV 330 nm.

### Reference Example 1

### Synthesis and Structural Analysis of 11-deacetyl pyripyropene O

Pyripyropene O (30 mg) was dissolved in methanol-water (19:1, 2 mL) and potassium carbonate (20 mg) was added thereto. The resultant was stirred for 22 hours, and thereafter acetic acid (0.1 mL) was added and the solvent was evaporated under reduced pressure. Ethyl acetate and water were added and then extraction was carried out with ethyl acetate. The ethyl acetate layer was washed with saturated sodium chloride solution and dried with anhydrous sodium sulfate. The solvent was evaporated under reduced pressure, thereby obtaining a crude product of 1,11-deacetyl pyripyropene O (30 mg). The crude product of 1,11-deacetyl pyripyropene O (23 mg) was dissolved in N,N-dimethylformamide (0.4 mL) and triethylamine (8 mg) and acetic acid anhydride (7 mg) were added thereto. After the resulting mixture was stirred at room temperature for 23 hours, water was added and then extraction was carried out with ethyl acetate. The ethyl acetate layer was washed with saturated sodium chloride solution and dried with anhydrous magnesium sulfate. The solvent was evaporated under reduced pressure, thereby obtaining a crude product of 1-deacetyl pyripyropene O (28 mg). The crude product of 1-deacetyl pyripyropene O (28 mg) was dissolved in toluene and 1,8-diazabicyclo [5,4,0]-7-undecene (20 mg) was added. The mixture was stirred at 70° C. for 20 hours and allowed to cool. Ethyl acetate and water were added and then extraction was carried out with ethyl acetate. The ethyl acetate layer was washed with saturated sodium chloride solution and dried with anhydrous magnesium sulfate. The solvent was evaporated under reduced pressure, thereby obtaining a crude product of 11-deacetyl pyripyropene O (20 mg).

After dissolved in methanol, this was used as a sample and HPLC (manufactured by SHIMADZU, LC-6AD, SPD-M20A PDA, CBM-20A, Column; Waters XTerra C18 (φ4.5×50 mm, 5 μm, mobile phase 30% aqueous acetonitrile solution to 55% aqueous acetonitrile solution in 25 minutes (linear gradient), flow rate: 1.0 ml/min, retention time 18 to 19 minutes) was repeated to preparative separation, thereby obtaining 11-deacetyl pyripyropene O (4.0 mg). ESI-MS; m/z 468 (M+H)⁺

¹H-NMR (CDCl₃) δ (ppm); 0.68 (3H, s), 0.95 (3H, s), 1.21-2.21 (10H, m), 1.25 (3H, s), 2.05 (3H, s), 2.20 (1H, dd, J=4.63, 17.3 Hz), 2.50 (1H, dd, J=4.63, 17.3 Hz), 2.94 (1H, d, J=12.5 Hz), 3.33 (1H, d, J=12.5 Hz), 4.87 (1H, dd, J=4.6, 12.2 Hz), 6.48 (1H, s), 7.57 (1H, dd, J=5.1, 8.1 Hz), 8.29 (1H, d, J=8.3 Hz), 8.68 (1H, d, J=4.6 Hz), 9.04 (1H, s)

### Reference Example 2

### Synthesis and Structural Analysis of Deacetyl Pyripyropene E

Pyripyropene E (29 mg) (Pyripyropene E was obtained by the method described in Japanese Patent Laid-Open Publication No. 239385/1996.) was dissolved in methanol-water (19:1, 1 mL) and potassium carbonate (53 mg) was added thereto. The resultant was stirred for 44 hours. Thereafter, the solvent was evaporated under reduced pressure and a mixed solvent of chloroform-methanol (10:1) was added. Insoluble matter was removed by filtration and the solvent was evaporated under reduced pressure, thereby obtaining a crude product. The crude product was purified by preparative thin layer chromatography (Merck silica gel 60F254, 0.5 mm, chloroform:methanol=10:1), thereby obtaining deacetyl pyripyropene E (18 mg).

ESI-MS; m/z 410 (M+H)⁺

¹H-NMR (CDCL₃) δ (ppm) 0.82 (3H, s), 0.92 (3H, s), 1.00-1.03 (1H, m), 1.04 (3H, s), 1.12 (1H, dt, J=4.0, 13.2 Hz), 1.27 (3H, s), 1.41-1.53 (2H, m), 1.59-1.75 (3H, m), 1.80-1.84 (2H, m), 2.15 (1H, dt, J=3.2, 12.4 Hz), 2.18-2.29 (1H, m), 2.54 (1H, dd, J=3.2, 17.6 Hz), 3.25 (1H, dd, J=4.4, 11.2 Hz), 6.43 (1H, s), 7.39 (1H, dd, J=4.8, 8.0 Hz), 8.11 (1H, d, J=8.0 Hz), 8.65 (1H, d, J=4.8 Hz), 8.99 (1H, d, J=1.6 Hz)

### Reference Example 3

### Synthesis of 7-Deacetyl Pyripyropene A

7-deacetyl pyripyropene A was synthesized by the method described in Japanese Patent Laid-Open Publication No. 259569/1996.

### Reference Example 4

### Obtainment of Pyripyropene E

Pyripyropene E was obtained by the method described in Japanese Patent Laid-Open Publication No. 239385/1996.

### Reference Example 5

### Obtainment of Pyripyropene O

Pyripyropene O was obtained by the method described in J. Antibiot. 1996, 49, 292.

### Reference Example 6

### Synthesis of pyripyropene A

Pyripyropene A was obtained by the method described in WO94/09147.

### [Accession Numbers]

FERM BP-11133

FERM BP-11137

FERM BP-11141

FERM BP-11219

FERM BP-11220

1. A method for producing a compound represented by the following formula A: 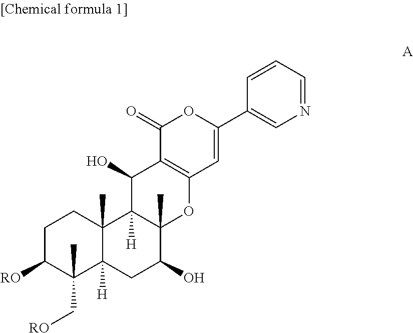
[wherein R represents a linear, branched or cyclic C₂₋₆ alkylcarbonyl group (when the alkyl moiety of this group is branched or cyclic, C₃₋₆ alkyl carbonyl group)],
the method comprising a step of culturing a microorganism into which at least one polynucleotide of (I) to (III) below or a recombinant vector comprising it/them is introduced with a compound represented by the following formula B: 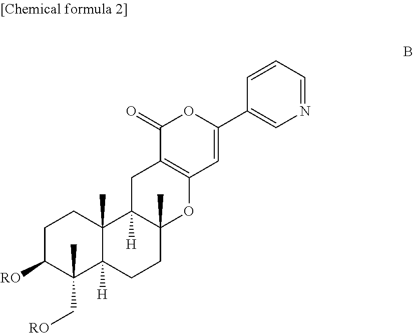
[wherein R represents the same meanings as above], and isolating the compound represented by formula A:
(I) an isolated polynucleotide having at least one nucleotide sequence selected from the nucleotide sequences of the following (a) to (d):
(a) a nucleotide sequence of SEQ ID NO:266,
(b) a nucleotide sequence which is capable of hybridizing with a sequence complementary to the nucleotide sequence of SEQ ID NO:266 under stringent conditions, and which encodes a protein substantially equivalent to the protein encoded by the nucleotide sequence of SEQ ID NO:266,
(c) a nucleotide sequence of SEQ ID NO:266 in which one or more nucleotides are deleted, substituted, inserted or added, and which encodes a protein substantially equivalent to the protein encoded by the each nucleotide sequence, and
(d) a nucleotide sequence which has at least 90% identity to the polynucleotide of the nucleotide sequence of SEQ ID NO:266, and which encodes a protein substantially equivalent to the protein encoded by the nucleotide sequence of SEQ ID NO:266;
(II) an isolated polynucleotide having a nucleotide sequence which encodes at least one amino acid sequence selected from SEQ ID NOs:269 and 270 or amino acid sequence substantially equivalent thereto; and
(III) an isolated polynucleotide having at least one nucleotide sequence selected from the nucleotide sequences of the following (1) to (4):
(1) a nucleotide sequence of the following (a) and (b):
(a) a nucleotide sequence from 13266 to 15144 of the nucleotide sequence shown in SEQ ID NO:266, and
(b) a nucleotide sequence from 16220 to 18018 of the nucleotide sequence shown in SEQ ID NO:266,
(2) a nucleotide sequence which is capable of hybridizing with a sequence complementary to the nucleotide sequence of (1) under stringent conditions, and which encodes a protein substantially equivalent to the protein encoded by the each nucleotide sequence,
(3) a nucleotide sequence of (1) in which one or more nucleotides are deleted, substituted, inserted or added, which encodes a protein substantially equivalent to the protein encoded by the each nucleotide sequence, and
(4) a nucleotide sequence which has at least 90% identity to the polynucleotide of the nucleotide sequence of (1), and which encodes a protein substantially equivalent to the protein encoded by the each nucleotide sequence.

2. The production method according to claim 1, wherein the compound represented by formula B according to claim 1 is a compound represented by the following formula B1:

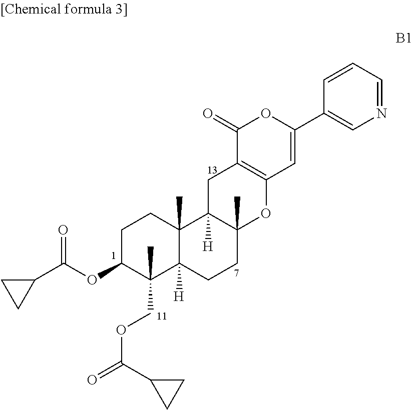

3. The production method according to claim 1, comprising a step of culturing a microorganism which comprises plasmid pCC1-PP1, plasmid pPP2 or plasmid pPP3, or one or more vectors selected from the group consisting of these plasmids with a compound represented by formula B according to claim 1 and isolating a compound represented by formula A according to claim 1.

4. The production method according to claim 1 or 2, comprising a step of culturing a microorganism into which at least one polynucleotide of (IV) to (V) below or a recombinant vector comprising it/them is introduced with a compound represented by formula B according to claim 1 and isolating a compound represented by formula A according to claim 1:
(IV) an isolated polynucleotide having a nucleotide sequence which encodes the amino acid sequence of SEQ ID NO:270 or an amino acid sequence substantially equivalent thereto; and
(V) an isolated polynucleotide having at least one nucleotide sequence selected from the nucleotide sequences of the following (1) to (4):
(1) a nucleotide sequence of the following (a),
(a) a nucleotide sequence from 16220 to 18018 of the nucleotide sequence shown in SEQ ID NO:266,
(2) a nucleotide sequence which is capable of hybridizing with a sequence complementary to the nucleotide sequence of (1) under stringent conditions, and which encodes a protein substantially equivalent to the protein encoded by the each nucleotide sequence,
(3) a nucleotide sequence of (1) in which one or more nucleotides are deleted, substituted, inserted or added, which encodes a protein substantially equivalent to the protein encoded by the each nucleotide sequence, and
(4) a nucleotide sequence which has at least 90% identity to the polynucleotide of the nucleotide sequence of (1), and which encodes a protein substantially equivalent to the protein encoded by the each nucleotide sequence.

5. The production method according to any one of claims 1 to 4, further comprising a step of acylating the hydroxyl groups at the 1 and 11 positions of a compound represented by the following formula D using an acylating agent: 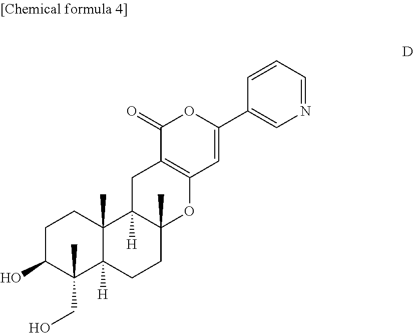
and isolating a compound represented by formula B according to claim 1.

6. The production method according to claim 5, further comprising a step of hydrolyzing the acetyl group at the 1 position and, when R′ is an acetyl group, the acetyl group at the 11 position of a compound represented by the following formula C: 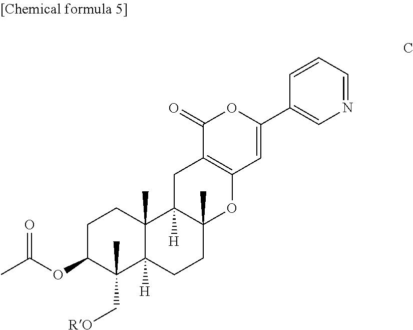
[wherein R′ represents an acetyl group or a hydrogen atom],
and isolating a compound represented by formula D according to claim 5.

7. The production method according to claim 6, further comprising a step of culturing a microorganism into which at least one polynucleotide of (VI) to (VII) below or a recombinant vector comprising it/them is introduced with pyripyropene E and isolating a compound represented by formula C according to claim 6:
(VI) an isolated polynucleotide having a nucleotide sequence which encodes at least one amino acid sequence selected from SEQ ID NOs:269 and 275;
(VII) an isolated polynucleotide having at least one nucleotide sequence selected from the nucleotide sequences of the following (1) to (4):
(1) a nucleotide sequence of the following (a) and (b),
(a) a nucleotide sequence from 13266 to 15144 of the nucleotide sequence shown in SEQ ID NO:266, and
(b) a nucleotide sequence from 25824 to 27178 of the nucleotide sequence shown in SEQ ID NO:266,
(2) a nucleotide sequence which is capable of hybridizing with a sequence complementary to the nucleotide sequence of (1) under stringent conditions, and which encodes a protein substantially equivalent to the protein encoded by the each nucleotide sequence,
(3) a nucleotide sequence of (1) in which one or more nucleotides are deleted, substituted, inserted or added, which encodes a protein substantially equivalent to the protein encoded by the each nucleotide sequence, and
(4) a nucleotide sequence which has at least 90% identity to the polynucleotide of the nucleotide sequence of (1), and which encodes a protein substantially equivalent to the protein encoded by the each nucleotide sequence.

8. The production method according to claim 7, comprising a step of culturing a microorganism which comprises plasmid pPP2 or plasmid pPP9 with pyripyropene E and isolating a compound represented by formula C according to claim 6.

9. The production method according to claim 6, further comprising a step of culturing a microorganism into which at least one polynucleotide of (VIII) to (IX) below or a recombinant vector comprising it/them is introduced with deacetyl pyripyropene E and isolating a compound represented by formula C according to claim 6:
(VIII) an isolated polynucleotide having a nucleotide sequence which encodes at least one amino acid sequence selected from SEQ ID NOs:269, 274 and 275 or amino acid sequence substantially equivalent thereto; and
(IX) an isolated polynucleotide having at least one nucleotide sequence selected from the nucleotide sequences of the following (1) to (4):
(1) a nucleotide sequence of the following (a), (b) and (c):
(a) a nucleotide sequence from 13266 to 15144 of the nucleotide sequence shown in SEQ ID NO:266,
(b) a nucleotide sequence from 23205 to 24773 of the nucleotide sequence shown in SEQ ID NO:266, and
(c) a nucleotide sequence from 25824 to 27178 of the nucleotide sequence shown in SEQ ID NO:266,
(2) a nucleotide sequence which is capable of hybridizing with a sequence complementary to the nucleotide sequence of (1) under stringent conditions, and which encodes a protein substantially equivalent to the protein encoded by the each nucleotide sequence,
(3) a nucleotide sequence of (1) in which one or more nucleotides are deleted, substituted, inserted or added, which encodes a protein substantially equivalent to the protein encoded by the each nucleotide sequence, and
(4) a nucleotide sequence which has at least 90% identity to the polynucleotide of the nucleotide sequence of (1), and which encodes a protein substantially equivalent to the protein encoded by the each nucleotide sequence.

10. The production method according to claim 9, comprising a step of culturing a microorganism which comprises plasmid pPP2 or plasmid pPP9 and also comprises plasmid pPP7 with deacetyl pyripyropene E and isolating a compound represented by formula C according to claim 6.

11. Use of plasmid pCC1-PP1, plasmid pPP2, plasmid pPP3, plasmid pPP7 or plasmid pPP9, or one or more vectors selected from the group consisting of these plasmids, for producing a compound represented by formula A.

12. Use of a transformant comprising plasmid pCC1-PP1, plasmid pPP2, plasmid pPP3, plasmid pPP7 or plasmid pPP9, or one or more vector selected from the group consisting of these plasmids, for producing a compound represented by formula A.

13. A compound represented by formula B1 according to claim 2.

14. A compound represented by formula D according to claim 5.
